(19) United States
(12) Patent Application Publication
(10) Pub. No.: US 2026/0028616 A1
(43) Pub. Date: Jan. 29, 2026

(54) METHODS AND APPARATUS FOR DUPLEX NUCLEOTIDE PURIFICATION

(71) Applicant: AGILENT TECHNOLOGIES, INC., Santa Clara, CA (US)

(72) Inventors: Joseph GUILES, Santa Clara, CA (US); Nevin GALLAGHER, Santa Clara, CA (US); Bob GRONEBERG, Santa Clara, CA (US)

(73) Assignee: AGILENT TECHNOLOGIES, INC., Santa Clara, CA (US)

(21) Appl. No.: 18/994,435

(22) PCT Filed: Sep. 22, 2022
(86) PCT No.: PCT/US2022/076891

§ 371 Date: Jan. 14, 2025

Related U.S. Application Data
(60) Provisional application No. 63/368,911, filed on Jul. 20, 2022.

(51) Int. Cl. C12N 15/10

(52) U.S. Cl. CPC C12N 15/1017

(57) ABSTRACT

Disclosed herein are methods of purifying oligonucleotides, for example, by separating double-stranded oligonucleotides from single-stranded oligonucleotides. Also provided are apparatuses for performed such methods. Also provided are double-stranded nucleic acid compositions and single-stranded nucleic acid compositions made using the methods and apparatuses disclosed herein. Also provided are methods of producing circular double-stranded nucleic acids.

### REFERENCE TO RELATED APPLICATIONS

The present application claims the priority benefit of U.S. provisional application No. 63/368,911, filed Jul. 20, 2022, the entire contents of which are incorporated herein by reference.

### BACKGROUND

Oligonucleotides are useful in a variety of biological processes in laboratory and industrial settings as well as in therapeutic applications. Manufacturing of oligonucleotides, e.g., at scale, industrially is time and resource intensive. There is a need for improvements and refinements in methods of manufacturing oligonucleotides that improve product and/or manufacturing characteristics.

### SUMMARY

The present disclosure pertains, at least in part, to methods and apparatuses that improve the manufacturing of oligonucleotides, improve the purification of oligonucleotides, and/or improve a characteristic (e.g., purity) of a product made by the methods or using the apparatuses. In some embodiments, the method improves the separation of double-stranded nucleic acids and single-stranded nucleic acids. In some embodiments, the methods improve the purity of a composition comprising double stranded nucleic acids. In some embodiments, the method comprises an ultrafiltration step that selectively retains double-stranded nucleic acids (e.g., nucleic acids comprising a first and second single-stranded nucleic acid). Without wishing to be bound by theory, it is believed that in some embodiments the methods and apparatuses retain the double-stranded nucleic acids in the retentate while allowing undesired mixture components to permeate the membrane.

Accordingly, in one aspect, the disclosure provides a method of manufacturing a purified double-stranded nucleic acid, comprising:
providing a mixture comprising:
   (a) a double-stranded nucleic acid comprising a first single-stranded nucleic acid hybridized to a complementary second single-stranded nucleic acid, and
   (b) at least one single-stranded nucleic acid, e.g., (i) the first single-stranded nucleic acid in single-stranded form (i.e., not hybridized to the second single-stranded nucleic acid), (ii) the second single-stranded nucleic acid in single-stranded form (i.e., not hybridized to the first single-stranded nucleic acid), or (iii) both;
subjecting the mixture to an ultra-filtration step that selectively retains the double-stranded nucleic acid comprising the hybridized first and second single-stranded nucleic acids but not the single-stranded nucleic acid(s) (e.g., the first and/or second single-stranded nucleic acids in single-stranded form); and
harvesting the retentate,
thereby manufacturing the purified double-stranded nucleic acid.

In another aspect, the disclosure provides a method of purifying a first and/or second single-stranded nucleic acid, comprising:
providing a mixture comprising:
   (a) a double-stranded nucleic acid comprising a first single-stranded nucleic acid hybridized to a complementary second single-stranded nucleic acid, and
   (b) at least one single-stranded nucleic acid, g., (i) the first single-stranded nucleic acid in single-stranded form (i.e., not hybridized to the second single-stranded nucleic acid), (ii) the second single-stranded nucleic acid in single-stranded form (i.e., not hybridized to the first single-stranded nucleic acid), or (iii) both;
subjecting the mixture to an ultra-filtration step that selectively retains the double-stranded nucleic acid comprising the hybridized first and second single-stranded nucleic acids but not the single-stranded nucleic acid (e.g., the first and/or second single-stranded nucleic acids in single-stranded form); and
and harvesting the permeate,
thereby purifying the first and/or second single-stranded nucleic acid.

In another aspect, the disclosure provides a method of separating a double-stranded nucleic acid from a single-stranded nucleic acid, comprising:
providing a mixture of double-stranded nucleic acid and single-stranded nucleic acid,
subjecting the mixture to an ultra-filtration step that selectively retains double-stranded nucleic acid and not single-stranded nucleic acid,
wherein the ultra-filtration step comprises applying the mixture to a filtration apparatus, e.g., a cross-flow filtration apparatus, with a membrane that has a molecular weight (MW) cutoff that is at least 10%, 20%, 30%, 40%, 50%, 60%, 70%, or 80% less than the MW of the double-stranded nucleic acid (e.g., at least 1, 2, 3, 4, 5, 6, 7, 8, 9, or 10 kilodaltons less than the MW of the double-stranded nucleic acid) and at least 10%, 20%, 30%, 40%, or 50% more than the MW of the single-stranded nucleic acid (e.g., at least 20, 15, 10, 9, 8, 7, 6, 5, 4, 3, or 2 kilodaltons more than the MW of the single-stranded nucleic acid),
thereby separating the double-stranded nucleic acid from the single-stranded nucleic acid.

In yet another aspect, the disclosure provides a method of separating a double-stranded nucleic acid from a single-stranded nucleic acid, comprising:
providing a mixture of double-stranded nucleic acid and single-stranded nucleic acid,
subjecting the mixture to an ultra-filtration step that selectively retains double-stranded nucleic acid and not single-stranded nucleic acid,
wherein the ultra-filtration step comprises applying the mixture to a filtration apparatus, e.g., a cross-flow filtration apparatus, with an average pore size (diameter) of at least 1, 1.2, 1.4, 1.6, 1.8, 2, 2.2, 2.4, 2.6, 2.8, or 3 nm and no more than 4, 3.8, 3.6, 3.4, 3.2, 3, 2.8, 2.6, 2.4, 2.2, 2, 1.8, or 1.6 nm,
thereby separating the double-stranded nucleic acid from the single-stranded nucleic acid.

In another aspect, the disclosure provides a method of separating a double-stranded nucleic acid from a single-stranded nucleic acid, comprising:
providing a mixture of double-stranded nucleic acid and single-stranded nucleic acid, and
subjecting the mixture to an ultra-filtration step that selectively retains double-stranded nucleic acid and not single-stranded nucleic acid,
wherein the ultra-filtration step comprises applying the mixture to a filtration apparatus, e.g., a cross-flow filtration apparatus, wherein the filtration apparatus comprises a membrane with an average pore size greater than Z (and optionally, less than 0.8Y, 0.85Y, 0.9Y, 0.95Y, 0.99Y, Y, or 1.1Y), wherein Z is the numerical average of X and Y,
wherein X is the smallest pore size that will allow at least 90% of each of the first single-stranded nucleic acid and/or second single-stranded nucleic acid to permeate the membrane (e.g., in less than about 2, 3, 4, 5, 6, 7, 8, 9, 10, 11, 12, 13, 14, 15, 20, 25, 27, 30, 35, 40, 45, 50, 55, 60, 63, 65, 70, 75, 80, 81, 85, or 90 diafiltration volumes (DTVs)); and
wherein Y is the largest pore size that will allow at least 90% of the double-stranded nucleic acid to be retained by the membrane (e.g., in less than about 2, 3, 4, 5, 6, 7, 8, 9, 10, 11, 12, 13, 14, 15, 20, 25, 27, 30, 35, 40, 45, 50, 55, 60, 63, 65, 70, 75, 80, 81, 85, or 90 DTVs),
thereby providing a method of separating the double-stranded nucleic acid from the single-stranded nucleic acid.

In another aspect, the disclosure provides a method of separating a double-stranded nucleic acid from a single-stranded nucleic acid, comprising:
providing a mixture of double-stranded nucleic acid and single-stranded nucleic acid, and
subjecting the mixture to an ultra-filtration step that selectively retains double-stranded nucleic acid and not single-stranded nucleic acid,
wherein the ultra-filtration step comprises applying the mixture to a filtration apparatus, e.g., a cross-flow filtration apparatus, wherein the filtration apparatus comprises a membrane with a pore size that will allow at least 90% of the single-stranded nucleic acids and at most 10% of the double-stranded nucleic acids to permeate the membrane (e.g., in about 2, 3, 4, 5, 6, 7, 8, 9, 10, 11, 12, 13, 14, 15, 20, 25, 27, 30, 35, 40, 45, 50, 55, 60, 63, 65, 70, 75, 80, 81, 85, or 90 DTVs).

In another aspect, the disclosure provides an apparatus comprising:
a first chamber configured to hold a mixture to be filtered (and optionally to capture retentate), a second chamber configured to capture permeate, and a filtration element disposed between the first and second chambers;
wherein the mixture comprises double-stranded nucleic acid (e.g., comprising a first single-stranded nucleic acid and a second single-stranded nucleic acid) and single-stranded nucleic acid (e.g., the first single-stranded nucleic acid in single-stranded form, the second single-stranded nucleic acid in single-stranded form, or both);
wherein the filtration element selectively retains double-stranded nucleic acid (e.g., comprising the first and second single-stranded nucleic acids) and not single-stranded nucleic acid (e.g., the first and/or second single-stranded nucleic acids in single-stranded form).

In some embodiments of any of the methods or apparatuses disclosed herein, the ultra-filtration step or filtration element also does not retain one or more other mixture component, e.g., a salt (e.g., a halide or mineral salt), buffer, or production reagent or byproduct. In certain embodiments, the other mixture component is chosen from: a shorter length single strand nucleic acid (e.g., shorter than the first and/or second single-stranded nucleic acid), an organic solvent (e.g., acetonitrile, ethanol, dimethylformamide, DMSO, toluene, pyridine, or lutidine), a thiolation reagent/bi-product (e.g., xanthane, hydride, or PADS), a capping reagent (e.g., acetic anhydride or NMI), a coupling reagent (e.g., phosphoramidite or ETT), a detritylation reagent/bi-product (e.g., dichloroacetic acid), a deprotection reagent/bi-product (e.g., diethylamine, methylamine, or ammonia), or a conjugation reagent (e.g carboxylic acid, carboxylic ester, carbonate, and carboxylic acid activator reagents).

In some embodiments of any of the methods or apparatuses disclosed herein, the ultra-filtration step or filtration element produces a retentate and a permeate. In certain embodiments, the retentate comprises a different concentration of one or more of a salt, a buffer, or a production reagent or byproduct than the permeate, the mixture, or both. In another embodiment, the retentate has a lower concentration of one or more of a salt, a buffer, or a production reagent or byproduct than the permeate, the mixture, or both.

In certain embodiments of any of the methods disclosed herein, providing a mixture comprises:
providing a first single-stranded nucleic acid and a second single-stranded nucleic acid which comprise sequences that are sufficiently complementary to one another to hybridize under conditions suitable for hybridization, e.g., as described in Examples 1-5, and
combining the first single-stranded nucleic acid and second single-stranded nucleic acid under conditions suitable for hybridization to produce a mixture comprising:
a double-stranded nucleic acid comprising a first single-stranded nucleic acid and a second single-stranded nucleic acid, and
the first single-stranded nucleic acid in single-stranded form, the second single-stranded nucleic acid in single-stranded form, or both.

In some embodiments, providing a first single-stranded nucleic acid and/or a second single-stranded nucleic acid comprises a synthesis step, e.g., comprising solid-state chemical synthesis, solution phase chemical synthesis, enzymatic synthesis, hybrid/chemical enzymatic synthesis, PCR-based synthesis, and cell synthesis of the first single-stranded nucleic acid, the second single-stranded nucleic acid, or both. In certain embodiments, providing a first single-stranded nucleic acid and/or a second single-stranded nucleic acid comprises a post-synthesis step, e.g., comprising conjugation, chemical or enzymatic cleavage from a solid substrate, removal of one or more chemical moieties (e.g., removal of a protecting group), or a combination thereof. In certain embodiments, the first single-stranded nucleic acid and/or second single-stranded nucleic acid may be conjugated during synthesis, after synthesis, or after single strand purification or ultrafiltration. In certain embodiments, providing a first single-stranded nucleic acid and/or a second single-stranded nucleic acid comprises a purification step, e.g., that enriches for nucleic acid components and decreases the level of non-nucleic acid components, e.g., a chromatography step, e.g., comprising anion exchange chromatography. In some embodiments, providing a first single-stranded nucleic acid and/or a second single-stranded nucleic acid comprises subjecting the first single-stranded nucleic acid, the second single-stranded nucleic acid, or both to an ultra-filtration step that selectively retains single-stranded nucleic acid (e.g., and not shortmers (i.e., shorter than full-length desired single-stranded nucleic acid) or non-nucleic acid components).

In some embodiments, providing a double-stranded nucleic acid comprises a synthesis step and/or a post-synthesis step, e.g., comprising conjugation of the double-strand nucleic acid to another component (e.g., nucleic acid, carbohydrate, peptide, protein domain, lipid, steroid polyethylene glycol, fluorescent label). In some embodiments, a single-stranded nucleic acid is conjugated to another component prior to formation of the double-stranded nucleic acid. In certain embodiments, providing a double-stranded nucleic acid enriches for nucleic acid components and decreases the level of non-nucleic acid components, e.g., a chromatography step, e.g., comprising anion exchange chromatography. In some embodiments, providing a first double-stranded nucleic acid and/or a conjugation component comprises subjecting the first double-stranded nucleic acid, the conjugation components, or both to an ultra-filtration step that selectively retains double-stranded nucleic acid (e.g., and not non-nucleic acid components).

In some embodiments of any of the methods or apparatuses disclosed herein, the first single-stranded nucleic acid is at least 10, 11, 12, 13, 14, 15, 16, 17, 18, 19, 20, 21, 22, 23, 24, 25, 26, 27, 28, 29, or 30 nucleotides long (and optionally no more than 100, 80 60, 50, 45, 40, 35, or 30 nucleotides long). In some embodiments, the second single-stranded nucleic acid is at least 10, 11, 12, 13, 14, 15, 16, 17, 18, 19, 20, 21, 22, 23, 24, 25, 26, 27, 28, 29, or 30 nucleotides long (and optionally no more than 100, 80, 60, 50, 45, 40, 35, or 30 nucleotides long). In certain embodiments, the double-stranded nucleic acid is at least 10, 11, 12, 13, 14, 15, 16, 17, 18, 19, 20, 21, 22, 23, 24, 25, 26, 27, 28, 29, or 30 base pairs long (and optionally no more than 100, 80, 60, 50, 45, 40, 35, or 30 base pairs long).

In some embodiments of any of the methods or apparatuses disclosed herein, the first single-stranded nucleic acid is 10-50, 12-50, 14-50, 16-50, 18-50, 20-50, 22-50, 24-50, 26-50, 28-50, 30-50, 32-50, 34-50, 36-50, 38-50, 40-50, 42-50, 44-50, 46-50, 48-50, 10-45, 12-45, 14-45, 16-45, 18-45, 20-45, 22-45, 24-45, 26-45, 28-45, 30-45, 32-45, 34-45, 36-45, 38-45, 40-45, 42-45, 10-40, 12-40, 14-40, 16-40, 18-40, 20-40, 22-40, 24-40, 26-40, 28-40, 30-40, 32-40, 34-40, 36-40, 38-40, 10-35, 12-35, 14-35, 16-35, 18-35, 20-35, 22-35, 24-35, 26-35, 28-35, 30-35, 32-35, 10-30, 12-30, 14-30, 16-30, 18-30, 20-30, 22-30, 24-30, 26-30, 28-30, 10-25, 12-25, 14-25, 16-25, 18-25, 20-25, 22-25, 10-20, 12-20, 14-20, 16-20, 18-20, 10-15, or 12-15 nucleotides long.

In certain embodiments of any of the methods or apparatuses disclosed herein, the second single-stranded nucleic acid is 10-50, 12-50, 14-50, 16-50, 18-50, 20-50, 22-50, 24-50, 26-50, 28-50, 30-50, 32-50, 34-50, 36-50, 38-50, 40-50, 42-50, 44-50, 46-50, 48-50, 10-45, 12-45, 14-45, 16-45, 18-45, 20-45, 22-45, 24-45, 26-45, 28-45, 30-45, 32-45, 34-45, 36-45, 38-45, 40-45, 42-45, 10-40, 12-40, 14-40, 16-40, 18-40, 20-40, 22-40, 24-40, 26-40, 28-40, 30-40, 32-40, 34-40, 36-40, 38-40, 10-35, 12-35, 14-35, 16-35, 18-35, 20-35, 22-35, 24-35, 26-35, 28-35, 30-35, 32-35, 10-30, 12-30, 14-30, 16-30, 18-30, 20-30, 22-30, 24-30, 26-30, 28-30, 10-25, 12-25, 14-25, 16-25, 18-25, 20-25, 22-25, 10-20, 12-20, 14-20, 16-20, 18-20, 10-15, or 12-15 nucleotides long.

In some embodiments of any of the methods or apparatuses disclosed herein, the double-stranded nucleic acid is 10-50, 12-50, 14-50, 16-50, 18-50, 20-50, 22-50, 24-50, 26-50, 28-50, 30-50, 32-50, 34-50, 36-50, 38-50, 40-50, 42-50, 44-50, 46-50, 48-50, 10-45, 12-45, 14-45, 16-45, 18-45, 20-45, 22-45, 24-45, 26-45, 28-45, 30-45, 32-45, 34-45, 36-45, 38-45, 40-45, 42-45, 10-40, 12-40, 14-40, 16-40, 18-40, 20-40, 22-40, 24-40, 26-40, 28-40, 30-40, 32-40, 34-40, 36-40, 38-40, 10-35, 12-35, 14-35, 16-35, 18-35, 20-35, 22-35, 24-35, 26-35, 28-35, 30-35, 32-35, 10-30, 12-30, 14-30, 16-30, 18-30, 20-30, 22-30, 24-30, 26-30, 28-30, 10-25, 12-25, 14-25, 16-25, 18-25, 20-25, 22-25, 10-20, 12-20, 14-20, 16-20, 18-20, 10-15, or 12-15 base pairs long.

In some embodiments of any of the methods or apparatuses disclosed herein, the double-stranded nucleic acid comprises a sequence having at least 75%, 80%, 85%, 90%, 95%, 96%, 97%, 98%, 99%, or 100% sequence identity to a nucleic acid sequence of interest (e.g., the sequence of a target gene, non-coding RNA, primer, or component for molecule assembly).

In certain embodiments of any of the methods or apparatuses disclosed herein, the first single-stranded nucleic acid is at least 3, 3.2, 3.4, 3.6, 3.8, 4, 4.2, 4.4, 4.6, 4.8, or 5 kilodaltons (and optionally no more than 10, 9, 8, 7, or 6 kilodaltons). In certain embodiments of any of the methods or apparatuses disclosed herein, the second single-stranded nucleic acid is at least 3, 3.2, 3.4, 3.6, 3.8, 4, 4.2, 4.4, 4.6, 4.8, or 5 kilodaltons (and optionally no more than 10, 9, 8, 7, or 6 kilodaltons).

In some embodiments of any of the methods or apparatuses disclosed herein, the first single-stranded nucleic acid is 3-20, 3-15, 3-10, 3.5-10, 4-10, 4.5-10, 5-10, 5.5-10, 6-10, 6.5-10, 7-10, 7.5-10, 8-10, 8.5-10, 9-10, 9.5-10, 3-9, 3.5-9, 4-9, 4.5-9, 5-9, 5.5-9, 6-9, 6.5-9, 7-9, 7.5-9, 8-9, 8.5-9, 3-8, 3.5-8, 4-8, 4.5-8, 5-8, 5.5-8, 6-8, 6.5-8, 7-8, 7.5-8, 3-7, 3.5-7, 4-7, 4.5-7, 5-7, 5.5-7, 6-7, 6.5-7, 3-6, 3.5-6, 4-6, 4.5-6, 5-6, 5.5-6, 3-5, 3.5-5, 4-5, 4.5-5, 3-4, or 3.5-4 kilodaltons.

In some embodiments of any of the methods or apparatuses disclosed herein, the second single-stranded nucleic acid is 3-20, 3-15, 3-10, 3.5-10, 4-10, 4.5-10, 5-10, 5.5-10, 6-10, 6.5-10, 7-10, 7.5-10, 8-10, 8.5-10, 9-10, 9.5-10, 3-9, 3.5-9, 4-9, 4.5-9, 5-9, 5.5-9, 6-9, 6.5-9, 7-9, 7.5-9, 8-9, 8.5-9, 3-8, 3.5-8, 4-8, 4.5-8, 5-8, 5.5-8, 6-8, 6.5-8, 7-8, 7.5-8, 3-7, 3.5-7, 4-7, 4.5-7, 5-7, 5.5-7, 6-7, 6.5-7, 3-6, 3.5-6, 4-6, 4.5-6, 5-6, 5.5-6, 3-5, 3.5-5, 4-5, 4.5-5, 3-4, or 3.5-4 kilodaltons.

In some embodiments of any of the methods or apparatuses disclosed herein, the first single-stranded nucleic acid comprises a sequence capable of hybridizing to itself, e.g., forming a hairpin loop, under conditions suitable for hybridization. In certain embodiments of any of the methods or apparatuses disclosed herein, the second single-stranded nucleic acid comprises a sequence capable of hybridizing to itself, e.g., forming a hairpin loop, under conditions suitable for hybridization.

In some embodiments of any of the methods or apparatuses disclosed herein, the first single-stranded nucleic acid does not comprise a sequence capable of hybridizing to itself, e.g., forming a hairpin loop, under conditions suitable for hybridization. In certain embodiments of any of the methods or apparatuses disclosed herein, the second single-stranded nucleic acid does not comprise a sequence capable of hybridizing to itself, e.g., forming a hairpin loop, under conditions suitable for hybridization.

In some embodiments of any of the methods or apparatuses disclosed herein, the first single-stranded nucleic acid, second single-stranded nucleic acid, or both are or comprise DNA, RNA, UNA, PNA, or LNA.

In certain embodiments of any of the methods or apparatuses disclosed herein, the first single-stranded nucleic acid, second single-stranded nucleic acid, or both comprise one or more modified and/or non-canonical nucleotides chosen from: MOE, 2′fluoro, 2′OMe, 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, 2-thiocytidine, methylated bases, intercalated bases, a nucleotide comprising a modified sugar (e.g., 2′-fluororibose, ribose, 2′-deoxyribose, arabinose, and hexose), or any combination thereof.

In some embodiments of any of the methods or apparatuses disclosed herein, the first single-stranded nucleic acid, second single-stranded nucleic acid, or both comprise one or more non-phosphodiester linkages between nucleotides, e.g., one or more phosphorothioate or 5′-N-phosphoramidate linkages.

In certain embodiments of any of the methods disclosed herein, the ultrafiltration step comprises applying the double-stranded nucleic acid product mixture to a filtration apparatus comprising a cross-flow filter. In some embodiments of any of the apparatuses disclosed herein, the filtration element comprises a cross-flow filter.

In some embodiments of any of the apparatuses disclosed herein, the filtration apparatus or filtration element comprises a membrane with a pore size that will allow at least 50%, 60%, 70%, 80%, or 90% of the single-stranded nucleic acids and at most 10%, 20%, 30%, 40%, or 50% of the double-stranded nucleic acids to permeate the membrane (e.g., in about 2, 3, 4, 5, 6, 7, 8, 9, 10, 11, 12, 13, 14, 15, 20, 25, 27, 30, 35, 40, 45, 50, 55, 60, 63, 65 70, 75, 80, 81, 85, or 90 DTVs).

In some embodiments of any of the apparatuses disclosed herein, the filtration apparatus or filtration element comprises a membrane with an average pore size greater than Z (and optionally, less than 0.8Y, 0.85Y, 0.9Y, 0.95Y, 0.99Y, Y, or 1.1Y), wherein Z is the numerical average of X and Y,
wherein X is the smallest pore size that will allow at least 90% of the a single-stranded nucleic acid, second single-stranded nucleic acid, or both to permeate the membrane (e.g., in less than about 2, 3, 4, 5, 6, 7, 8, 9, 10, 11, 12, 13, 14, 15, 20, 25, 27, 30, 35, 40, 45 50, 55, 60, 63,65, 70, 75, 80, 81, 85, or 90 diafiltration volumes (DTVs)); and
wherein Y is the largest pore size that will allow at least 90% of the double-stranded nucleic acid to be retained by the membrane (e.g., in less than about 2, 3, 4, 5, 6, 7, 8, 9, 10, 11, 12, 13, 14, 15, 20, 25, 27, 30, 35, 40, 45, 50, 55, 60, 63,65, 70, 75, 80, 81, 85, or 90 DTVs).

In certain embodiments of any of the apparatuses disclosed herein, the filtration apparatus or filtration element comprises a membrane with an average pore size of 0.05-20, 0.1-20, 0.5-20, 1-20, 2-20, 3-20, 4-20, 5-20, 6-20, 7-20, 8-20, 9-20, 10-20, 15-20, 0.05-10, 0.1-10, 0.5-10, 1-10, 2-10, 3-10, 4-10, 5-10, 6-10, 7-10, 8-10, 9-10, 0.05-8, 0.1-8, 0.5-8, 1-8, 2-8, 3-8, 4-8, 5-8, 6-8, 7-8, 0.05-6, 0.1-6, 0.5-6, 1-6, 2-6, 3-6, 4-6, 5-6, 0.05-4, 0.1-4, 0.5-4, 1-4, 2-4, 3-4, 0.05-2, 0.1-2, 0.5-2, 1-2, 0.05-1, 0.1-1, or 0.5-1 μm.

In some embodiments of any of the apparatuses disclosed herein, the filtration apparatus or filtration element comprises a membrane with a molecular weight cutoff of:
at least 1, 2, 3, 4, 5, 6, 7, 8, 9, 10, 15, 20, 25, or 30 kilodaltons greater than the molecular weight of the single-stranded nucleic acid (e.g., the first single-stranded nucleic acid, the second single-stranded nucleic acid, or both), and
at least 1, 2, 3, 4, 5, 6, 7, 8, 9, 10, 15, 20, 25, or 30 kilodaltons less than the molecular weight of the double-stranded nucleic acid.

In another embodiment of any of the apparatuses disclosed herein, the filtration apparatus or filtration element comprises a membrane with a molecular weight cutoff of 1-30, 2-30, 3-30, 4-30, 5-30, 6-30, 7-30, 8-30, 9-30, 10-30, 15-30, 20-30, 25-30, 1-25, 2-25, 3-25, 4-25, 5-25, 6-25, 7-25, 8-25, 9-25, 10-25, 15-25, 20-25, 1-20, 2-20, 3-20, 4-20, 5-20, 6-20, 7-20, 8-20, 9-20, 10-20, 15-20, 1-15, 2-15, 3-15, 4-15, 5-15, 6-15, 7-15, 8-15, 9-15, 10-15, 1-10, 2-10, 3-10, 4-10, 5-10, 6-10, 7-10, 8-10, 9-10, 1-9, 2-9, 3-9, 4-9, 5-9, 6-9, 7-9, 8-9, 1-8, 2-8, 3-8, 4-8, 5-8, 6-8, 7-8, 1-7, 2-7, 3-7, 4-7, 5-7, 6-7, 1-6, 2-6, 3-6, 4-6, 5-6, 1-5, 2-5, 3-5, 4-5, 1-4, 2-4, 3-4, 1-3, 2-3, or 1-2 kilodaltons greater than the molecular weight of the single-stranded nucleic acid (e.g., the first single-stranded nucleic acid, the second single-stranded nucleic acid, or both).

In certain embodiments of any of the apparatuses disclosed herein, the filtration apparatus or filtration element comprises a membrane with a molecular weight cutoff of 1-30, 2-30, 3-30, 4-30, 5-30, 6-30, 7-30, 8-30, 9-30, 10-30, 15-30, 20-30, 25-30, 1-25, 2-25, 3-25, 4-25, 5-25, 6-25, 7-25, 8-25, 9-25, 10-25, 15-25, 20-25, 1-20, 2-20, 3-20, 4-20, 5-20, 6-20, 7-20, 8-20, 9-20, 10-20, 15-20, 1-15, 2-15, 3-15, 4-15, 5-15, 6-15, 7-15, 8-15, 9-15, 10-15, 1-10, 2-10, 3-10, 4-10, 5-10, 6-10, 7-10, 8-10, 9-10, 1-9, 2-9, 3-9, 4-9, 5-9, 6-9, 7-9, 8-9, 1-8, 2-8, 3-8, 4-8, 5-8, 6-8, 7-8, 1-7, 2-7, 3-7, 4-7, 5-7, 6-7, 1-6, 2-6, 3-6, 4-6, 5-6, 1-5, 2-5, 3-5, 4-5, 1-4, 2-4, 3-4, 1-3, 2-3, or 1-2 kilodaltons less than the molecular weight of the double-stranded nucleic acid.

In some embodiments of any of the apparatuses disclosed herein, the filtration apparatus or filtration element comprises a membrane with a molecular weight cutoff of:
at least 2 kilodaltons greater than the molecular weight of the single-stranded nucleic acid (e.g., the first single-stranded nucleic acid, the second single-stranded nucleic acid, or both), and
at least 5 kilodaltons less than the molecular weight of the double-stranded nucleic acid.

In certain embodiments of any of the apparatuses disclosed herein, the filtration apparatus or filtration element comprises a membrane with a molecular weight cutoff of 0.5-100, 0.5-80, 0.5-60, 0.5-50, 0.5-40, 0.5-30, 0.5-20, 0.5-15, 0.5-10, 0.5-8, 0.5-6, 0.5-4, 0.5-3, 0.5-2, 0.5-1, 1-100, 1-80, 1-60, 1-50, 1-40, 1-30, 1-20, 1-15, 1-10, 1-8, 1-6, 1-4, 1-3, 1-2, 2-100, 2-80, 2-60, 2-50, 2-40, 2-30, 2-20, 2-15, 2-10, 2-8, 2-6, 2-4, 2-3, 3-100, 3-80, 3-60, 3-50, 3-40, 3-30, 3-20, 3-15, 3-10, 3-8, 3-6, 3-4, 4-100, 4-80, 4-60, 4-50, 4-40, 4-30, 4-20, 4-15, 4-10, 4-8, 4-6, 6-100, 6-80, 6-60, 6-50, 6-40, 6-30, 6-20, 6-15, 6-10, 6-8, 8-100, 8-80, 8-60, 8-50, 8-40, 8-30, 8-20, 8-15, 8-10, 10-100, 10-80, 10-60, 10-50, 10-40, 10-30, 10-20, 10-15, 15-100, 15-80, 15-60, 15-50, 15-40, 15-30, 15-20, 20-100, 20-80, 20-60, 20-50, 20-40, 20-30, 30-100, 30-80, 30-60, 30-50, 30-40, 40-100, 40-80, 40-60, 40-50, 50-100, 50-80, 50-60, 60-80, 60-100, or 80-100 kilodaltons.

In some embodiments of any of the apparatuses disclosed herein, the filtration apparatus or filtration element comprises a membrane with a molecular weight cutoff of about 1-2, 2-3, 3-4, 4-5, 5-6, 6-7, 7-8, 8-9, 9-10, 10-11, 11-12, 12-13, 13-14, 14-15, 15-16, 16-17, 17-18, 18-19, 19-20, 20-21, 21-22, 22-23, 23-24, 24-25, 25-26, 26-27, 27-28, 28-29, 29-30, 30-31, 25-35, 50, 100, 300, or 1,000 kDa. In certain embodiments, the filtration apparatus or filtration element comprises a membrane with a molecular weight cutoff of about 8-9, 9-10, 10-11, 11-12, 12-13, 13-14, or 14-15 kDa. In some embodiments, the filtration apparatus or filtration element comprises a membrane with a molecular weight cutoff of about 8-15 kDa. In certain embodiments, the filtration apparatus or filtration element comprises a membrane with a molecular weight cutoff of about 10 kDa.

In some embodiments of any of the methods disclosed herein, the ultrafiltration step comprises applying a force of at least 5, 6, 7, 8, 9, 10, 11, 12, 13, 14, 15, 16, 17, 18, 19, 20, 21, 22, 23, 24, 25, 26, 27, 28, or 29 psi (transmembrane pressure) and optionally no more than 30, 29, 28, 27, 26, 25, 24, 23, 22, 21, 20, 19, 18, 17, 16, 15, 14, 13, 12, 11, 10, 9, 8, 7, or 6 psi to the double-stranded nucleic acid product mixture, e.g., within the filtration apparatus. In certain embodiments, the ultrafiltration step comprises applying a force of 5-30, 10-30, 15-30, 20-30, 25-30, 5-25, 10-25, 15-25, 20-25, 5-20, 10-20, 15-20, 5-15, 10-15, or 5-10 psi (transmembrane pressure). In another embodiment, the ultrafiltration step comprises applying a force within the filtration apparatus' manufacturer's recommended pressure operating conditions. In some embodiments, the ultrafiltration step comprises applying at least 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, or 90 diafiltration volumes (DTVs) to the filtration apparatus (e.g., sequentially) and optionally no more than 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, or 90 DTVs. In some embodiments, the ultrafiltration step comprises applying 20-90, 25-90, 30-90, 35-90, 40-90, 45-90, 50-90, 55-90, 60-90, 65-90, 70-90, 75-90, 80-90, 85-90, 20-85, 25-85, 30-85, 35-85, 40-85, 45-85, 50-85, 55-85, 60-85, 65-85, 70-85, 75-85, 80-85, 20-80, 25-80, 30-80, 35-80, 40-80, 45-80, 50-80, 55-80, 60-80, 65-80, 70-80, 75-80, 20-75, 25-75, 30-75, 35-75, 40-75, 45-75, 50-75, 55-75, 60-75, 65-75, 70-75, 20-70, 25-70, 30-70, 35-70, 40-70, 45-70, 50-70, 55-70, 60-70, 65-70, 20-65, 25-65, 30-65, 35-65, 40-65, 45-65, 50-65, 55-65, 60-65, 20-60, 25-60, 30-60, 35-60, 40-60, 45-60, 50-60, 55-60, 20-55, 25-55, 30-55, 35-55, 40-55, 45-55, 50-55, 20-50, 25-50, 30-50, 35-50, 40-50, 45-50, 20-45, 25-45, 30-45, 35-45, 40-45, 20-40, 25-40, 30-40, 35-40, 20-35, 25-35, 30-35, 20-30, 25-30, or 20-25 DTVs to the filtration apparatus (e.g., sequentially). In some embodiments, the ultrafiltration step comprises achieving a recirculation rate under pressure of at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39 L/min/m² and optionally no more than 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, or 6 L/min/m². In certain embodiments, the ultrafiltration step comprises achieving a recirculation rate under pressure of 5-40, 10-40, 15-40, 20-40, 25-40, 30-40, 35-40, 5-35, 10-35, 15-35, 20-35, 25-35, 30-35, 5-30, 10-30, 15-30, 20-30, 25-30, 5-25, 10-25, 15-25, 20-25 5-20, 10-20, 15-20, 5-15, 10-15, or 5-10 L/min/m². In some embodiments, the ultrafiltration is performed until a steady low conductivity threshold is achieved (e.g., a conductivity threshold of less than or equal to about 30, 40, 50, 60, 70, or 80 μS/cm, e.g., about 50 μS/cm).

In certain embodiments of any of the apparatuses disclosed herein, the filtration apparatus or filtration element membrane comprises polysulfone, polypropylene, cellulose acetate, polyactic acid, nitrocellulose, mix cellulose ester cellulose nitrate, regenerated cellulose, polyethersulfone, polyamide, cellulose derivative (Hydrosart®) polyvinylidene fluoride, polytetra-fluoroethylene, or polycarbonate track etched membranes. In some embodiments, the filtration apparatus or filtration element is or comprises a hollow fiber, tubular, spiral-wound, cassette, plate, or frame membrane type.

In some embodiments of any of the methods disclosed herein, the method manufactures purified double-stranded nucleic acid that achieves a standard for purity, and wherein the standard for purity comprises the retentate comprising at least 80, 85, 90, 91, 92, 93, 94, 95, 96, 97, 98, or 99% double-stranded nucleic acid (e.g., and comprising less than 20, 15, 10, 9, 8, 7, 6, 5, 4, 3, 2, or 1% single-stranded nucleic acid). In certain embodiments, the method manufactures purified double-stranded nucleic acid that achieves a standard for purity, and wherein the standard for purity comprises the retentate comprising 80-100, 85-100, 90-100, 91-100, 92-100, 93-100, 94-100, 95-100, 96-100, 97-100, 98-100, 99-100, 80-99, 85-99, 90-99, 91-99, 92-99, 93-99, 94-99, 95-99, 96-99, 97-99, or 98-99% double-stranded nucleic acid. In certain embodiments, the method manufactures purified double-stranded nucleic acid that achieves a standard for purity, and wherein the standard for purity comprises the retentate comprising 1-20, 1-15, 1-10, 1-9, 1-8, 1-7, 1-6, 1-5, 1-4, 1-3, 1-2, 1-1, 2-1, 3-1, 4-1, 5-1, 6-1, 7-1, 8-1, 9-1, 10-1, 15-1, or 20-1 of single-stranded nucleic acid to double-stranded nucleic acid.

In some embodiments of any of the methods disclosed herein, the method achieves the standard for purity in no more than 25, 24, 23, 22, 21, or 20 DTVs.

In some embodiments of any of the methods or apparatuses disclosed herein, the length of at least one strand of the double-stranded nucleic acid and the length of the single-stranded nucleic acid are the same.

In some embodiments of any of the methods or apparatuses disclosed herein, the single-stranded nucleic acid is a component of the double-stranded nucleic acid (e.g., the double-stranded nucleic acid comprises a copy of the single-stranded nucleic acid).

In some embodiments of any of the methods or apparatuses disclosed herein, the double-stranded nucleic acid comprises a blunt end. In some embodiments of any of the methods or apparatuses disclosed herein, the double-stranded nucleic acid comprises two blunt ends. In certain embodiments of any of the methods or apparatuses disclosed herein, the double-stranded nucleic acid comprises a single-stranded portion, e.g., a terminal overhang, e.g., on one end. In some embodiments of any of the methods or apparatuses disclosed herein, the double-stranded nucleic acid has a single-stranded portion, e.g., a terminal overhang, on both ends. In certain embodiments of any of the methods or apparatuses disclosed herein, the double-stranded nucleic acid comprises a conjugate group, e.g., that is or comprises PEG (polyethylene glycol), a dye, a label, a peptide, a lipid, a steroid, a carbohydrate, e.g., GalNAc, or a glycol spacer.

In some embodiments of any of the apparatuses disclosed herein, the second chamber comprises permeate, e.g., comprising single-stranded nucleic acid. In other embodiments, the second chamber does not retain permeate, e.g., the permeate flows from the second chamber and is discarded.

In certain embodiments of any of the apparatuses disclosed herein, the first chamber comprises the mixture. In certain embodiments of any of the methods or apparatuses disclosed herein, the first chamber comprises the retentate.

In some embodiments of any of the methods or apparatuses disclosed herein, the mixture comprises no more than 1, 2, 3, 4, 5, 6, 7, 8, 9, 10, 20, 30, 40, 50, 60, 70, 80, 90, 100 μl, 200 μl, 300 μl, or 400 μl, or no more than 0.5, 1, 2, 3, 4, 5, 6, 7, 8, 9, 10, 20, 30, 40, 50, 60, 70, 80, 90, 100 ml, 200 ml, 300 ml, or 400 ml, or no more than 0.5, 1, 2, 3, 4, 5, 6, 7, 8, 9, 10, 20, 30, 40, 50, 60, 70, 80, 90, 100, 150, 200, 250, 300, 350, 400, 450, or 500 L (and optionally, at least 1, 2, 3, 4, 5, 6, 7, 8, 9, 10, 20, 30, 40, 50, 60, 70, 80, 90, or 100 μl, or at least 0.5, 1, 2, 3, 4, 5, 6, 7, 8, 9, 10, 20, 30, 40, 50, 60, 70, 80, 90, or 100 ml, or at least 0.5, 1, 2, 3, 4, 5, 6, 7, 8, 9, 10, 20, 30, 40, 50, 60, 70, 80, 90, 100, 150, 200, 250, 300, 350, 400, 450, or 500 L).

In some embodiments of any of the methods or apparatuses disclosed herein, the mixture comprises at least 1, 2, 3, 4, 5, 6, 7, 8, 9, 10, 20, 30, 40, 50, 60, 70, 80, 90, 100 μl, 200 μl, 300 μl, or 400 μl, or at least 0.5, 1, 2, 3, 4, 5, 6, 7, 8, 9, 10, 20, 30, 40, 50, 60, 70, 80, 90, 100 ml, 200 ml, 300 ml, or 400 ml, or at least 0.5, 1, 2, 3, 4, 5, 6, 7, 8, 9, 10, 20, 30, 40, 50, 60, 70, 80, 90, 100, 150, 200, 250, 300, 350, 400, 450, or 500 L (and optionally no more than 1, 2, 3, 4, 5, 6, 7, 8, 9, 10, 20, 30, 40, 50, 60, 70, 80, 90, or 100 μl, or no more than 0.5, 1, 2, 3, 4, 5, 6, 7, 8, 9, 10, 20, 30, 40, 50, 60, 70, 80, 90, or 100 ml, or no more than 0.5, 1, 2, 3, 4, 5, 6, 7, 8, 9, 10, 20, 30, 40, 50, 60, 70, 80, 90, 100, 150, 200, 250, 300, 350, 400, 450, or 500 L).

In certain embodiments of any of the methods or apparatuses disclosed herein, the mixture comprises:
1-100, 5-100, 10-100, 20-100, 30-100, 40-100, 50-100, 60-100, 70-100, 80-100, 90-100, 1-50, 5-50, 10-50, 20-50, 30-50, 40-50, 1-20, 5-20, 10-20, or 1-10 μl,
0.1-100, 0.5-100, 1-100, 5-100, 10-100, 20-100, 30-100, 40-100, 50-100, 60-100, 70-100, 80-100, 90-100, 0.1-50, 0.5-50, 1-50, 5-50, 10-50, 20-50, 30-50, 40-50, 0.1-20, 0.5-20, 1-20, 5-20, 10-20, 0.1-10, 0.5-10, 1-10, 0.1-1, 0.5-1, or 0.1-0.5 ml, or
0.1-500, 0.5-500, 1-500, 5-500, 10-500, 20-500, 30-500, 40-500, 50-500, 60-500, 70-500, 80-500, 90-500, 100-500, 150-500, 200-500, 250-500, 300-500, 350-500, 400-500, 450-500, 0.1-250, 0.5-250, 1-250, 5-250, 10-250, 20-250, 30-250, 40-250, 50-250, 60-250, 70-250, 80-250, 90-250, 100-250, 150-250, 200-250, 0.1-100, 0.5-100, 1-100, 5-100, 10-100, 20-100, 30-100, 40-100, 50-100, 60-100, 70-100, 80-100, 90-100, 0.1-50, 0.5-50, 1-50, 5-50, 10-50, 20-50, 30-50, 40-50, 0.1-20, 0.5-20, 1-20, 5-20, 10-20, 0.1-10, 0.5-10, 1-10, 0.1-1, 0.5-1, or 0.1-0.5 L.

In some embodiments of any of the methods or apparatuses disclosed herein, the mixture comprises a nucleic acid concentration (e.g., overall nucleic acid concentration, double-stranded nucleic acid concentration, or single-stranded nucleic acid concentration) of at least 300, 400, 500, 600, 700, 800, 900, 1000, 1100, 1200, 1300, 1400, 1500, 1600, 1700, 1800, 1900, or 2000 OD/ml (e.g., OD at 250-260 nm, e.g., 258-260 nm) (and optionally no more than 2500, 2000, 1500, 1400, 1300, 1200, 1100, or 1000 OD/ml). In certain embodiments, the mixture comprises a nucleic acid concentration (e.g., overall nucleic acid concentration, double-stranded nucleic acid concentration, or single-stranded nucleic acid concentration) of at least about 900 OD/mL (e.g., about 900 OD/mL). In some embodiments, the mixture comprises a nucleic acid concentration (e.g., overall nucleic acid concentration, double-stranded nucleic acid concentration, or single-stranded nucleic acid concentration) of at least about 1100 OD/mL (e.g., about 1100 OD/mL). In certain embodiments, the mixture comprises a nucleic acid concentration (e.g., overall nucleic acid concentration, double-stranded nucleic acid concentration, or single-stranded nucleic acid concentration) of at least about 400 OD/mL (e.g., about 400 OD/mL).

In certain embodiments of any of the methods or apparatuses disclosed herein, the ssDNA in the permeate is composed primarily of sense strands (e.g., wherein at least 75%, 80%, 85%, 90%, 95%, 96%, 97%, 98%, 99%, 99.5%, 99.9%, or 100% of the ssDNA in the permeate consists of sense strands). In some embodiments, the ssDNA in the permeate is composed primarily of antisense strands (e.g., wherein at least 75%, 80%, 85%, 90%, 95%, 96%, 97%, 98%, 99%, 99.5%, 99.9%, or 100% of the ssDNA in the permeate consists of antisense strands).

In some embodiments of any of the methods or apparatuses disclosed herein, the double-stranded nucleic acid comprises a mismatch (e.g., at one of the ends of the double-stranded nucleic acid, within 1-2 nucleotides of one of the ends of the double stranded nucleic acid, or between the ends of the double-stranded nucleic acids).

In certain embodiments of any of the methods or apparatuses disclosed herein, the mixture comprises an amount of the single-stranded nucleic acid in excess of the amount of the double-stranded nucleic acid. In some embodiments, the amount of the single-stranded nucleic acid is in excess relative to the amount of the double-stranded nucleic acid by at least about 1%, 2%, 3%, 4%, 5%, 6%, 7%, 8%, 9%, 10%, 15%, 20%, 25%, 30%, 40%, 50%, 60%, 70%, 80%, or 90%. In certain embodiments, the amount of the single-stranded nucleic acid is in excess relative to the amount of the double-stranded nucleic acid by about 5%.

In some embodiments of any of the methods disclosed herein, the ultra-filtration step comprises diafiltration. In certain embodiments, the diafiltration is performed for at least 10, 20, 30, 40, or 50 minutes, or for at least 1, 2, 3, 4, 5, 6, 7, 8, 9, 10, 11, 12, 13, 14, 15, 16, 17, 18, 19, 20, 21, 22, 23, or 24 hours. In some embodiments, the diafiltration is performed for a period sufficient to filter at least about 2, 3, 4, 5, 6, 7, 8, 9, 10, 11, 12, 13, 14, 15, 20, 25, 27, 30, 35, 40, 45, 50, 55, 60, 63,65, 70, 75, 80, 81, 85, or 90 diafiltration volumes (DTVs).

In certain embodiments of any of the methods disclosed herein, the ultra-filtration is applied at a pressure of about 5-40, 10-40, 15-40, 20-40, 30-40, 35-40, 5-15, 5-20, 5-25, 5-30, 5-35, 10-30, 15-25, 5-10, 10-15, 15-20, 20-25, 25-30, 30-35, or 35-40 psi. In some embodiments, the ultra-filtration is applied at a pressure of about 15-30 psi (e.g., about 15-25, 15-20, 25-30, 20-30, 15-20, 20-25, or 25-30 psi, e.g., about 15, 16, 17, 18, 19, 20, 21, 22, 23, 24, 25, 26, 27, 28, 29, or 30 psi). In certain embodiments, the ultra-filtration is performed at a temperature of about 10-40° C., e.g., about 15-40, 20-40, 25-40, 30-40, 35-40, 10-35, 10-30, 10-25, 10-20, 10-15, 15-35, 20-30, 10-15, 15-20, 20-25, 25-30, 30-35, or 35-40° C., e.g., about 10, 15, 20, 25, 30, 35, 37, or 40° C.

In some embodiments of any of the methods disclosed herein, the method further comprises analyzing the retentate by non-denaturing HPLC (e.g., to determine the relative quantity of double-stranded nucleic acid and/or single-stranded nucleic acid in the retentate), e.g., as described in Examples 1-5.

In some embodiments of any of the methods or apparatuses disclosed herein, the ratio of double-stranded nucleic acid relative to single-stranded nucleic acid in the retentate (e.g., as determined by non-denaturing HPLC) increases over time and/or over diafiltration total volume (DTV). In certain embodiments, the ratio of double-stranded nucleic acid relative to single-stranded nucleic acid in the retentate (e.g., as determined by non-denaturing HPLC) is at least 90:5, 90.3:5, 90.4:4.8, 90.9:4.8, 91.1:4.6, 91.2:4.5, 91.1:4.4, 91.2:4.4, 91.3:4.1, or 91.5:3.9. In some embodiments, the ratio of double-stranded nucleic acid relative to single-stranded nucleic acid in the retentate (e.g., as determined by non-denaturing HPLC) is at least 90.1:5.3, 91.1:4.7, 91.2:4.8, 91.3:4.9, 92.0:3.5, 92.1:3.4, or 92.0:3.2.

In some embodiments of any of the methods or apparatuses disclosed herein, the concentration of a salt (e.g., a cation, e.g., sodium ion) in the mixture is less than about 10, 20, 30, 40, 50, 60, 70, 80, 90, 100, 200, 300, 400, 500, 600, 700, 800, 900, or 1000 mM. In certain embodiments, the mixture does not substantially comprise the salt. In certain embodiments, the mixture is essentially free of the salt.

In some embodiments of any of the methods or apparatuses disclosed herein, the concentration of a salt (e.g., a cation, e.g., sodium ion) in the mixture is about 50 mM to about 1.5M, e.g., about 60 nM to about 1.2 M, or about 69 mM to about 1.02 M.

In certain embodiments of any of the methods or apparatuses disclosed herein, the melting temperature of the double-stranded nucleic acid in the mixture is between about 20-85° C., e.g., between about 25-80, 30-75, 35-70, 40-65, 45-60, 20-25, 25-30, 30-35, 35-40, 40-45, 45-50, 50-55, 55-60, 60-65, 65-70, 70-75, 75-80, or 80-85° C.

In certain embodiments of any of the methods or apparatuses disclosed herein, the relationship between the melting temperature of the double-stranded nucleic acid in the mixture and the concentration of a salt (e.g., a cation, e.g., sodium ion) in the mixture is determined according to a method as described in Owczarzy et al. (2004, Biochemistry 43:3537-3554; incorporated herein by reference in its entirety).

In certain embodiments of any of the methods or apparatuses disclosed herein, the double-stranded nucleic acid of the retentate is for use in a downstream use or application. In some embodiments, the double-stranded nucleic acid of the retentate is lyophilized. In some embodiments, the double-stranded nucleic acid of the retentate is conjugated with an additional agent (e.g., an additional nucleic acid molecule). In certain embodiments, the double-stranded nucleic acid of the retentate is mixed with other oligonucleotide duplexes. In certain embodiments, the double-stranded nucleic acid of the retentate is mixed with a drug product formulation buffer, e.g., buffered phosphate solution, buffered phosphate saline solution, saline solution, or water).

In some embodiments of any of the methods or apparatuses disclosed herein, the double-stranded nucleic acid has a length of 5-10, 10-15, 15-20, 20-25, 25-30, 30-40, 40-50, 50-60, 60-70, 70-80, 80-90, 90-100, 100-125, 125-150, 150-200, 200-250, 250-300, 300-400, or 400-500 nucleotides.

In certain embodiments of any of the methods disclosed herein, the method reduces the level of low-molecular weight impurities by at least 25, 50, 75, 80, 85, 90, 95, 96, 97, 98, 99, or 100%. In some embodiments, the low-molecular weight impurities each have a molecular weight of less than about 500, 400, 300, 200, 100, 50, 40, 30, 20, or 10 Daltons. In certain embodiments, the low-molecular weight impurities comprise salts and/or trace solvents.

In some embodiments of any of the methods or apparatuses disclosed herein, the method reduces the level of the double-stranded nucleic acid by no more than 0.1, 0.5, 1, 2, 3, 4, 5, 6, 7, 8, 9, or 10%.

In some embodiments of any of the methods or apparatuses disclosed herein, the ratio of the concentration of the double-stranded nucleic acid in the retentate and the concentration of the double-stranded nucleic acid in the mixture is at least 0.75, 0.8, 0.85, 0.9, 0.95, 0.96, 0.97, 0.98, 0.99, 0.995, or 0.999.

In some embodiments of any of the methods or apparatuses disclosed herein, the quantity of the double-stranded nucleic acid in the retentate is at least 20, 22, 24, 26, 28, 30, 32, 34, 36, 38, 40, 42, 44, 46, 48, or 50 mg/mL.

In another aspect, the disclosure provides a method of optimizing a technique for manufacturing a purified double-stranded nucleic acid, comprising:
(i) providing a mixture comprising:
   a double-stranded nucleic acid comprising a first single-stranded nucleic acid and a second single-stranded nucleic acid at a first concentration, and
   a single-stranded nucleic acid, e.g., the first single-stranded nucleic acid in single-stranded form, the second single-stranded nucleic acid in single-stranded form at a second concentration, or both;
   wherein the mixture has a predetermined concentration of a salt (e.g., a cation, e.g., sodium ion);
(ii) subjecting the mixture to an ultra-filtration step that selectively retains double-stranded nucleic acid comprising the first and second single-stranded nucleic acids and not the single-stranded nucleic acid (e.g., the first and second single-stranded nucleic acids in single-stranded form),
   wherein the ultra-filtration step comprises applying the mixture to a filtration apparatus with a predetermined molecular weight cutoff for a predetermined length of time;
(iii) harvesting the retentate;
(iv) varying one of or more of the first concentration, the second concentration, the predetermined molecular weight cutoff, the predetermined length of time, or the salt concentration;
(v) repeating steps (i)-(iii) one or more times, each using one or more of the varied first concentration, second concentration, predetermined molecular weight cutoff, predetermined length of time, and/or salt concentration;
(vi) determining, for each repetition of steps (i)-(iii), the ratio of double-stranded nucleic acid to single-stranded nucleic acid in the harvested retentates; and
(vii) identifying a first concentration, the second concentration, predetermined molecular weight cutoff, predetermined length of time, and/or salt concentration that yields the highest ratio of double-stranded nucleic acid to single-stranded nucleic acid in the harvested retentates;
thereby optimizing the technique for manufacturing the purified double-stranded nucleic acid.

In another aspect, the disclosure provides a double-stranded nucleic acid composition comprising a harvested retentate produced according to a method described herein.

In another aspect, the disclosure provides a single-stranded nucleic acid composition comprising a harvested permeate produced according to a method described herein.

In another aspect, the disclosure provides a composition comprising a first double-stranded nucleic acid comprising a first overhang hybridized to a second double-stranded nucleic acid comprising a second overhang (e.g., complementary to the first overhang).

In some embodiments, the first double-stranded nucleic acid is made according to a method described herein. In certain embodiments, the second double-stranded nucleic acid is made according to a method described herein.

In another aspect, the disclosure provides a method of producing a circular double-stranded nucleic acid, comprising:
(a) providing a composition described herein, and
(b) incubating the composition under conditions suitable for circularization of the first double-stranded nucleic acid hybridized to the second double-stranded nucleic acid.

In some embodiments, the circularization comprises ligation.

The various embodiments disclosed herein are represented by the following clauses:
1. A method of manufacturing a double-stranded nucleic acid, comprising:
   providing a mixture comprising:
      a double-stranded nucleic acid comprising a first single-stranded nucleic acid and a second single-stranded nucleic acid, and
      a single-stranded nucleic acid;
   subjecting the mixture to an ultra-filtration step that selectively retains double-stranded nucleic acid comprising the hybridized first and second single-stranded nucleic acids and not the single-stranded nucleic acid; and
   harvesting the retentate,
thereby manufacturing a purified double-stranded nucleic acid.
2. A method of purifying a first and/or second single-stranded nucleic acid, comprising:
   providing a mixture comprising:
      a double-stranded nucleic acid comprising a first single-stranded nucleic acid
      and a second single-stranded nucleic acid, and
      a single-stranded nucleic acid,
      optionally wherein the mixture has been subjected to a preliminary purification/ultrafiltration to remove shortmers and/or organic impurities resulting from the any nucleic acid synthesis and/or deprotection steps that were performed;
   subjecting the mixture to an ultra-filtration step that selectively retains double-stranded nucleic acid comprising the first and second single-stranded nucleic acids and not the single-stranded nucleic acid; and
   and harvesting the permeate,
thereby purifying a first and/or second single-stranded nucleic acid.
3. A method of separating a double-stranded nucleic acid from a single-stranded nucleic acid, comprising:
   providing a mixture of double-stranded nucleic acid and single-stranded nucleic acid,
   subjecting the mixture to an ultra-filtration step that selectively retains double-stranded nucleic acid and not single-stranded nucleic acid,
   wherein the ultra-filtration step comprises applying the mixture to a filtration apparatus, with a molecular weight (MW) cutoff that is at least 1, 2, 3, 4, 5, 6, 7, 8, 9, or 10 kilodaltons less than the MW of the double-stranded nucleic acid and no more than 20, 15, 10, 9, 8, 7, 6, 5, 4, 3, or 2 kilodaltons less than the MW of the double-stranded nucleic acid
   thereby separating a double-stranded nucleic acid from a single-stranded nucleic acid.
4. A method of separating a double-stranded nucleic acid from a single-stranded nucleic acid, comprising:
   providing a mixture of double-stranded nucleic acid and single-stranded nucleic acid, subjecting the mixture to an ultra-filtration step that selectively retains double-stranded
   nucleic acid and not single-stranded nucleic acid,
   wherein the ultra-filtration step comprises applying the mixture to a filtration apparatus, with an average pore size (diameter) of at least 1, 1.2, 1.4, 1.6, 1.8, 2, 2.2, 2.4, 2.6, 2.8, or 3 nm and no more than 4, 3.8, 3.6, 3.4, 3.2, 3, 2.8, 2.6, 2.4, 2.2, 2, 1.8, or 1.6 nm,
   thereby separating a double-stranded nucleic acid from a single-stranded nucleic acid.
5. A method of separating a double-stranded nucleic acid from a single-stranded nucleic acid, comprising:
   providing a mixture of double-stranded nucleic acid and single-stranded nucleic acid, and
   subjecting the mixture to an ultra-filtration step that selectively retains double-stranded nucleic acid and not single-stranded nucleic acid,
   wherein the ultra-filtration step comprises applying the mixture to a filtration apparatus, wherein the filtration apparatus comprises a membrane with an average pore size greater than Z, and optionally less than 0.8Y, 0.85Y, 0.9Y, 0.95Y, 0.99Y, Y, or 1.1Y, wherein Z is the numerical average of X and Y,
   wherein X is the smallest pore size that will allow at least 90% of the first single-stranded nucleic acid, second single-stranded nucleic acid, or both to permeate the membrane; and
   wherein Y is the largest pore size that will allow at least 90% of the double-stranded nucleic acid to be retained by the membrane,
   thereby separating a double-stranded nucleic acid from a single-stranded nucleic acid.
6. An apparatus comprising:
   a first chamber configured to hold a mixture to be filtered, and optionally to capture retentate, a second chamber configured to capture permeate, and a filtration element disposed between the first and second chambers;
   wherein the mixture comprises double-stranded nucleic acid and single-stranded nucleic acid;
   wherein the filtration element selectively retains double-stranded nucleic acid and not single-stranded nucleic acid.
7. The method or apparatus of any of the preceding clauses, wherein the ultra-filtration step or filtration element also does not retain one or more other mixture component.
8. The method or apparatus of clause 7, wherein the other mixture component is chosen from: a shorter length single strand nucleic acid, an organic solvent, a thiolation reagent/by-product, a capping reagent, a coupling reagent, a detritylation reagent/by-product, a conjugation reagent (e.g., carboxylic acid, carboxylic ester, carbonate, and carboxylic acid activator reagents), and/or a deprotection reagent/by-product.
9. The method of any one of clauses 1-5, wherein the ultra-filtration step produces a retentate and a permeate.
10. The method or apparatus of any of clauses 6-9, wherein the retentate comprises a different concentration of one or more of a salt, a buffer, a deprotection base, or a production reagent or byproduct than the permeate, the mixture, or both.
11. The method or apparatus of any of clauses 6-10, wherein the retentate has a lower concentration of one or more of a salt, a buffer, or a production reagent or byproduct than the permeate, the mixture, or both.
12. The method of any of the preceding clauses, wherein providing a mixture comprises:
   providing a first single-stranded nucleic acid and a second single-stranded nucleic acid which comprise sequences that are sufficiently complementary to one another to hybridize under conditions suitable for hybridization, and
   combining the first single-stranded nucleic acid and second single-stranded nucleic acid under conditions suitable for hybridization to produce a mixture comprising:
      a double-stranded nucleic acid comprising a first single-stranded nucleic acid and a second single-stranded nucleic acid, and
      the first single-stranded nucleic acid in single-stranded form, the second single-stranded nucleic acid in single-stranded form, or both.
13. The method of clause 12, wherein providing a first single-stranded nucleic acid and a second single-stranded nucleic acid comprises a synthesis step.
14. The method of either of clauses 12 or 13, wherein providing a first single-stranded nucleic acid and a second single-stranded nucleic acid comprises a post-synthesis step.
15. The method of any of clauses 12-14, wherein providing a first single-stranded nucleic acid and a second single-stranded nucleic acid comprises a purification step.
16. The method of any of clauses 12-15, wherein providing a first single-stranded nucleic acid and a second single-stranded nucleic acid comprises a subjecting the first single-stranded nucleic acid, the second single-stranded nucleic acid, or both to an ultra-filtration step that selectively retains single-stranded nucleic acid.
17. The method or apparatus of any of the preceding clauses, wherein the first single-stranded nucleic acid is at least 10, 11, 12, 13, 14, 15, 16, 17, 18, 19, 20, 21, 22, 23, 24, 25, 26, 27, 28, 29, or 30 nucleotides long, and optionally no more than 100, 80, 60, 50, 45, 40, 35, or 30 nucleotides long.
18. The method or apparatus of any of the preceding clauses, wherein the second single-stranded nucleic acid is at least 10, 11, 12, 13, 14, 15, 16, 17, 18, 19, 20, 21, 22, 23, 24, 25, 26, 27, 28, 29, or 30 nucleotides long, and optionally no more than 100, 80, 60, 50, 45, 40, 35, or 30 nucleotides long.
19. The method or apparatus of any of the preceding clauses, wherein the double-stranded nucleic acid is at least 10, 11, 12, 13, 14, 15, 16, 17, 18, 19, 20, 21, 22, 23, 24, 25, 26, 27, 28, 29, or 30 base pairs long, and optionally no more than 100, 80, 60, 50, 45, 40, 35, or 30 base pairs long.
20. The method or apparatus of any of the preceding clauses, wherein the first single-stranded nucleic acid is 10-50, 12-50, 14-50, 16-50, 18-50, 20-50, 22-50, 24-50, 26-50, 28-50, 30-50, 32-50, 34-50, 36-50, 38-50, 40-50, 42-50, 44-50, 46-50, 48-50, 10-45, 12-45, 14-45, 16-45, 18-45, 20-45, 22-45, 24-45, 26-45, 28-45, 30-45, 32-45, 34-45, 36-45, 38-45, 40-45, 42-45, 10-40, 12-40, 14-40, 16-40, 18-40, 20-40, 22-40, 24-40, 26-40, 28-40, 30-40, 32-40, 34-40, 36-40, 38-40, 10-35, 12-35, 14-35, 16-35, 18-35, 20-35, 22-35, 24-35, 26-35, 28-35, 30-35, 32-35, 10-30, 12-30, 14-30, 16-30, 18-30, 20-30, 22-30, 24-30, 26-30, 28-30, 10-25, 12-25, 14-25, 16-25, 18-25, 20-25, 22-25, 10-20, 12-20, 14-20, 16-20, 18-20, 10-15, or 12-15 nucleotides long.
21. The method or apparatus of any of the preceding clauses, wherein the second single-stranded nucleic acid is 10-50, 12-50, 14-50, 16-50, 18-50, 20-50, 22-50, 24-50, 26-50, 28-50, 30-50, 32-50, 34-50, 36-50, 38-50, 40-50, 42-50, 44-50, 46-50, 48-50, 10-45, 12-45, 14-45, 16-45, 18-45, 20-45, 22-45, 24-45, 26-45, 28-45, 30-45, 32-45, 34-45, 36-45, 38-45, 40-45, 42-45, 10-40, 12-40, 14-40, 16-40, 18-40, 20-40, 22-40, 24-40, 26-40, 28-40, 30-40, 32-40, 34-40, 36-40, 38-40, 10-35, 12-35, 14-35, 16-35, 18-35, 20-35, 22-35, 24-35, 26-35, 28-35, 30-35, 32-35, 10-30, 12-30, 14-30, 16-30, 18-30, 20-30, 22-30, 24-30, 26-30, 28-30, 10-25, 12-25, 14-25, 16-25, 18-25, 20-25, 22-25, 10-20, 12-20, 14-20, 16-20, 18-20, 10-15, or 12-15 nucleotides long.
22. The method or apparatus of any of the preceding clauses, wherein the double-stranded nucleic acid is 10-50, 12-50, 14-50, 16-50, 18-50, 20-50, 22-50, 24-50, 26-50, 28-50, 30-50, 32-50, 34-50, 36-50, 38-50, 40-50, 42-50, 44-50, 46-50, 48-50, 10-45, 12-45, 14-45, 16-45, 18-45, 20-45, 22-45, 24-45, 26-45, 28-45, 30-45, 32-45, 34-45, 36-45, 38-45, 40-45, 42-45, 10-40, 12-40, 14-40, 16-40, 18-40, 20-40, 22-40, 24-40, 26-40, 28-40, 30-40, 32-40, 34-40, 36-40, 38-40, 10-35, 12-35, 14-35, 16-35, 18-35, 20-35, 22-35, 24-35, 26-35, 28-35, 30-35, 32-35, 10-30, 12-30, 14-30, 16-30, 18-30, 20-30, 22-30, 24-30, 26-30, 28-30, 10-25, 12-25, 14-25, 16-25, 18-25, 20-25, 22-25, 10-20, 12-20, 14-20, 16-20, 18-20, 10-15, or 12-15 base pairs long.
23. The method or apparatus of any of the preceding clauses, wherein the double-stranded nucleic acid comprises a sequence having at least 75%, 80%, 85%, 90%, 95%, 96%, 97%, 98%, 99%, or 100% sequence identity to a nucleic acid sequence of interest.
24. The method or apparatus of any of the preceding clauses, wherein the first single-stranded nucleic acid is at least 3, 3.2, 3.4, 3.6, 3.8, 4, 4.2, 4.4, 4.6, 4.8, 5, 5.2, 5.4, 5.6, 5.8, 6, 6.2, 6.4, 6.6, 6.8, 7, 7.2, 7.4, 7.6, 7.8. 8, 8.2, 8.4, 8.6, 8.8, 9, 9.2, 9.4, 9.6, 9.8, or 10 kilodaltons, and optionally no more than 15, 14, 13, 12, 11, 10, 9, 8, 7, or 6 kilodaltons.
25. The method or apparatus of any of the preceding clauses, wherein the second single-stranded nucleic acid is at least 3, 3.2, 3.4, 3.6, 3.8, 4, 4.2, 4.4, 4.6, 4.8, 5, 5.2, 5.4, 5.6, 5.8, 6, 6.2, 6.4, 6.6, 6.8, 7, 7.2, 7.4, 7.6, 7.8. 8, 8.2, 8.4, 8.6, 8.8, 9, 9.2, 9.4, 9.6, 9.8, or 10 kilodaltons, and optionally no more than 15, 14, 13, 12, 11, 10, 9, 8, 7, or 6 kilodaltons.
26. The method or apparatus of any of the preceding clauses, wherein the first single-stranded nucleic acid is 3-10, 3.5-10, 4-10, 4.5-10, 5-10, 5.5-10, 6-10, 6.5-10, 7-10, 7.5-10, 8-10, 8.5-10, 9-10, 9.5-10, 3-9, 3.5-9, 4-9, 4.5-9, 5-9, 5.5-9, 6-9, 6.5-9, 7-9, 7.5-9, 8-9, 8.5-9, 3-8, 3.5-8, 4-8, 4.5-8, 5-8, 5.5-8, 6-8, 6.5-8, 7-8, 7.5-8, 3-7, 3.5-7, 4-7, 4.5-7, 5-7, 5.5-7, 6-7, 6.5-7, 3-6, 3.5-6, 4-6, 4.5-6, 5-6, 5.5-6, 3-5, 3.5-5, 4-5, 4.5-5, 3-4, or 3.5-4 kilodaltons.
27. The method or apparatus of any of the preceding clauses, wherein the second single-stranded nucleic acid is 3-10, 3.5-10, 4-10, 4.5-10, 5-10, 5.5-10, 6-10, 6.5-10, 7-10, 7.5-10, 8-10, 8.5-10, 9-10, 9.5-10, 3-9, 3.5-9, 4-9, 4.5-9, 5-9, 5.5-9, 6-9, 6.5-9, 7-9, 7.5-9, 8-9, 8.5-9, 3-8, 3.5-8, 4-8, 4.5-8, 5-8, 5.5-8, 6-8, 6.5-8, 7-8, 7.5-8, 3-7, 3.5-7, 4-7, 4.5-7, 5-7, 5.5-7, 6-7, 6.5-7, 3-6, 3.5-6, 4-6, 4.5-6, 5-6, 5.5-6, 3-5, 3.5-5, 4-5, 4.5-5, 3-4, or 3.5-4 kilodaltons.
28. The method or apparatus of any of the preceding clauses, wherein the first single-stranded nucleic acid comprises a sequence capable of hybridizing to itself under conditions suitable for hybridization.
29. The method or apparatus of any of the preceding clauses, wherein the second single-stranded nucleic acid comprises a sequence capable of hybridizing to itself under conditions suitable for hybridization.
30. The method or apparatus of any of clauses 1-27 or 29, wherein the first single-stranded nucleic acid does not comprise a sequence capable of hybridizing to itself under conditions suitable for hybridization.
31. The method or apparatus of any of clauses 1-28 or 30, wherein the second single-stranded nucleic acid does not comprise a sequence capable of hybridizing to itself under conditions suitable for hybridization.
32. The method or apparatus of any of the preceding clauses, wherein the first single-stranded nucleic acid, second single-stranded nucleic acid, or both are or comprise DNA, RNA, UNA, PNA, or LNA.
33. The method or apparatus of any of the preceding clauses, wherein the first single-stranded nucleic acid, second single-stranded nucleic acid, or both comprise one or more modified and/or non-canonical nucleotides chosen from: MOE, 2′fluoro, 2′OMe, 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, 2-thiocytidine, methylated bases, intercalated bases, a nucleotide comprising a modified sugar, or any combination thereof.
34. The method or apparatus of any of the preceding clauses, wherein the first single-stranded nucleic acid, second single-stranded nucleic acid, or both comprise one or more non-phosphodiester linkages between nucleotides.
35. The method of any of the preceding clauses, wherein the ultrafiltration step comprises applying the double-stranded nucleic acid product mixture to a filtration apparatus comprising a cross-flow filter.
36. The apparatus of any of the preceding clauses, wherein the filtration element comprises a cross-flow filter.
37. The method or apparatus of any of the preceding clauses, wherein the filtration apparatus or filtration element comprises a membrane with an average pore size greater than Z, and optionally less than 0.8Y, 0.85Y, 0.9Y, 0.95Y, 0.99Y, Y, or 1.1Y, wherein Z is the numerical average of X and Y,
   wherein X is the smallest pore size that will allow at least 90% of the first single-stranded nucleic acid, second single-stranded nucleic acid, or both to permeate the membrane; and
   wherein Y is the largest pore size that will allow at least 90% of the double-stranded nucleic acid to by retained by the membrane.
38. The method or apparatus of any of the preceding clauses, wherein the filtration apparatus or filtration element comprises a membrane with an average pore size of 0.05-20, 0.1-20, 0.5-20, 1-20, 2-20, 3-20, 4-20, 5-20, 6-20, 7-20, 8-20, 9-20, 10-20, 15-20, 0.05-10, 0.1-10, 0.5-10, 1-10, 2-10, 3-10, 4-10, 5-10, 6-10, 7-10, 8-10, 9-10, 0.05-8, 0.1-8, 0.5-8, 1-8, 2-8, 3-8, 4-8, 5-8, 6-8, 7-8, 0.05-6, 0.1-6, 0.5-6, 1-6, 2-6, 3-6, 4-6, 5-6, 0.05-4, 0.1-4, 0.5-4, 1-4, 2-4, 3-4, 0.05-2, 0.1-2, 0.5-2, 1-2, 0.05-1, 0.1-1, or 0.5-1 μm.
39. The method or apparatus of any of the preceding clauses, wherein the filtration apparatus or filtration element comprises a membrane with a molecular weight cutoff of:
   at least 1, 2, 3, 4, 5, 6, 7, 8, 9, 10, 15, 20, 25, or 30 kilodaltons greater than the molecular weight of the single-stranded nucleic acid, and
   at least 1, 2, 3, 4, 5, 6, 7, 8, 9, 10, 15, 20, 25, or 30 kilodaltons less than the molecular weight of the double-stranded nucleic acid.
40. The method or apparatus of any of the preceding clauses, wherein the filtration apparatus or filtration element comprises a membrane with a molecular weight cutoff of 1-30, 2-30, 3-30, 4-30, 5-30, 6-30, 7-30, 8-30, 9-30, 10-30, 15-30, 20-30, 25-30, 1-25, 2-25, 3-25, 4-25, 5-25, 6-25, 7-25, 8-25, 9-25, 10-25, 15-25, 20-25, 1-20, 2-20, 3-20, 4-20, 5-20, 6-20, 7-20, 8-20, 9-20, 10-20, 15-20, 1-15, 2-15, 3-15, 4-15, 5-15, 6-15, 7-15, 8-15, 9-15, 10-15, 1-10, 2-10, 3-10, 4-10, 5-10, 6-10, 7-10, 8-10, 9-10, 1-9, 2-9, 3-9, 4-9, 5-9, 6-9, 7-9, 8-9, 1-8, 2-8, 3-8, 4-8, 5-8, 6-8, 7-8, 1-7, 2-7, 3-7, 4-7, 5-7, 6-7, 1-6, 2-6, 3-6, 4-6, 5-6, 1-5, 2-5, 3-5, 4-5, 1-4, 2-4, 3-4, 1-3, 2-3, or 1-2 kilodaltons greater than the molecular weight of the single-stranded nucleic acid.
41. The method or apparatus of any of the preceding clauses, wherein the filtration apparatus or filtration element comprises a membrane with a molecular weight cutoff of 1-30, 2-30, 3-30, 4-30, 5-30, 6-30, 7-30, 8-30, 9-30, 10-30, 15-30, 20-30, 25-30, 1-25, 2-25, 3-25, 4-25, 5-25, 6-25, 7-25, 8-25, 9-25, 10-25, 15-25, 20-25, 1-20, 2-20, 3-20, 4-20, 5-20, 6-20, 7-20, 8-20, 9-20, 10-20, 15-20, 1-15, 2-15, 3-15, 4-15, 5-15, 6-15, 7-15, 8-15, 9-15, 10-15, 1-10, 2-10, 3-10, 4-10, 5-10, 6-10, 7-10, 8-10, 9-10, 1-9, 2-9, 3-9, 4-9, 5-9, 6-9, 7-9, 8-9, 1-8, 2-8, 3-8, 4-8, 5-8, 6-8, 7-8, 1-7, 2-7, 3-7, 4-7, 5-7, 6-7, 1-6, 2-6, 3-6, 4-6, 5-6, 1-5, 2-5, 3-5, 4-5, 1-4, 2-4, 3-4, 1-3, 2-3, or 1-2 kilodaltons less than the molecular weight of the double-stranded nucleic acid.
42. The method or apparatus of any of the preceding clauses, wherein the filtration apparatus or filtration element comprises a membrane with a molecular weight cutoff of:
   greater than the molecular weight of the single-stranded nucleic acid, and
   less than the molecular weight of the double-stranded nucleic acid.
43. The method or apparatus of any of the preceding clauses, wherein the filtration apparatus or filtration element comprises a membrane with a molecular weight cutoff of 0.5-100, 0.5-80, 0.5-60, 0.5-50, 0.5-40, 0.5-30, 0.5-20, 0.5-15, 0.5-10, 0.5-8, 0.5-6, 0.5-4, 0.5-3, 0.5-2, 0.5-1, 1-100, 1-80, 1-60, 1-50, 1-40, 1-30, 1-20, 1-15, 1-10, 1-8, 1-6, 1-4, 1-3, 1-2, 2-100, 2-80, 2-60, 2-50, 2-40, 2-30, 2-20, 2-15, 2-10, 2-8, 2-6, 2-4, 2-3, 3-100, 3-80, 3-60, 3-50, 3-40, 3-30, 3-20, 3-15, 3-10, 3-8, 3-6, 3-4, 4-100, 4-80, 4-60, 4-50, 4-40, 4-30, 4-20, 4-15, 4-10, 4-8, 4-6, 6-100, 6-80, 6-60, 6-50, 6-40, 6-30, 6-20, 6-15, 6-10, 6-8, 8-100, 8-80, 8-60, 8-50, 8-40, 8-30, 8-20, 8-15, 8-10, 10-100, 10-80, 10-60, 10-50, 10-40, 10-30, 10-20, 10-15, 15-100, 15-80, 15-60, 15-50, 15-40, 15-30, 15-20, 20-100, 20-80, 20-60, 20-50, 20-40, 20-30, 30-100, 30-80, 30-60, 30-50, 30-40, 40-100, 40-80, 40-60, 40-50, 50-100, 50-80, 50-60, 60-80, 60-100, or 80-100 kilodaltons.
44. The method or apparatus of any of the preceding clauses, wherein the filtration apparatus or filtration element comprises a membrane with a molecular weight cutoff of about 2-3, 3-4, 4-5, 5-6, 6-7, 7-8, 8-9, 9-10, 10-11, 11-12, 12-13, 13-14, 14-15, or 15-16 kDa.
45. The method or apparatus of any of the preceding clauses, wherein the filtration apparatus or filtration element comprises a membrane with a molecular weight cutoff of about 8-9, 9-10, 10-11, 11-12, 12-13, 13-14, or 14-15 kDa.
46. The method or apparatus of any of the preceding clauses, wherein the filtration apparatus or filtration element comprises a membrane with a molecular weight cutoff of about 8-15 kDa.
47. The method or apparatus of any of the preceding clauses, wherein the filtration apparatus or filtration element comprises a membrane with a molecular weight cutoff of about 10 kDa.
48. The method of any of the preceding clauses, wherein the ultrafiltration step comprises applying a force of at least 5, 6, 7, 8, 9, 10, 11, 12, 13, 14, 15, 16, 17, 18, 19, 20, 21, 22, 23, 24, 25, 26, 27, 28, or 29 psi (transmembrane pressure) and optionally no more than 30, 29, 28, 27, 26, 25, 24, 23, 22, 21, 20, 19, 18, 17, 16, 15, 14, 13, 12, 11, 10, 9, 8, 7, or 6 psi to the double-stranded nucleic acid product mixture.
49. The method of any of the preceding clauses, wherein the ultrafiltration step comprises applying a force of 5-30, 10-30, 15-30, 20-30, 25-30, 5-25, 10-25, 15-25, 20-25, 5-20, 10-20, 15-20, 5-15, 10-15, or 5-10 psi (transmembrane pressure).
50. The method of any of the preceding clauses, wherein the ultrafiltration step comprises applying a force within the filtration apparatus' manufacturer's recommended pressure operating conditions.
51. The method of any of the preceding clauses, wherein the ultrafiltration step comprises applying at least 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, or 90 diafiltration volumes (DTVs) to the filtration apparatus and optionally no more than 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, or 90 DTVs.
52. The method of any of the preceding clauses, wherein the ultrafiltration step comprises applying 20-90, 25-90, 30-90, 35-90, 40-90, 45-90, 50-90, 55-90, 60-90, 65-90, 70-90, 75-90, 80-90, 85-90, 20-85, 25-85, 30-85, 35-85, 40-85, 45-85, 50-85, 55-85, 60-85, 65-85, 70-85, 75-85, 80-85, 20-80, 25-80, 30-80, 35-80, 40-80, 45-80, 50-80, 55-80, 60-80, 65-80, 70-80, 75-80, 20-75, 25-75, 30-75, 35-75, 40-75, 45-75, 50-75, 55-75, 60-75, 65-75, 70-75, 20-70, 25-70, 30-70, 35-70, 40-70, 45-70, 50-70, 55-70, 60-70, 65-70, 20-65, 25-65, 30-65, 35-65, 40-65, 45-65, 50-65, 55-65, 60-65, 20-60, 25-60, 30-60, 35-60, 40-60, 45-60, 50-60, 55-60, 20-55, 25-55, 30-55, 35-55, 40-55, 45-55, 50-55, 20-50, 25-50, 30-50, 35-50, 40-50, 45-50, 20-45, 25-45, 30-45, 35-45, 40-45, 20-40, 25-40, 30-40, 35-40, 20-35, 25-35, 30-35, 20-30, 25-30, or 20-25 DTVs to the filtration apparatus.
53. The method of any of the preceding clauses, wherein the ultrafiltration step comprises achieving a recirculation rate under pressure of at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39 L/min/m² and optionally no more than 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, or 6 L/min/m².
54. The method of any of the preceding clauses, wherein the ultrafiltration step comprises achieving a recirculation rate under pressure of 5-40, 10-40, 15-40, 20-40, 25-40, 30-40, 35-40, 5-35, 10-35, 15-35, 20-35, 25-35, 30-35, 5-30, 10-30, 15-30, 20-30, 25-30, 5-25, 10-25, 15-25, 20-25, 5-20, 10-20, 15-20, 5-15, 10-15, or 5-10 L/min/m².
55. The method of any of the preceding clauses, wherein the ultrafiltration is performed until a steady low conductivity threshold is achieved.
56. The method or apparatus of any of the preceding clauses, wherein the filtration apparatus or filtration element membrane comprises polysulfone, polypropylene, cellulose acetate, polyactic acid, nitrocellulose, mix cellulose ester cellulose nitrate, regenerated cellulose, polyethersulfone, polyamide, cellulose derivative (Hydrosart®) polyvinylidene fluoride, polytetra-fluoroethylene, or polycarbonate track etched membranes.
57. The method or apparatus of any of the preceding clauses, wherein the filtration apparatus or filtration element is or comprises a hollow fiber, tubular, spiral-wound, cassette, plate, or frame membrane type.
58. The method of any of the preceding clauses, wherein the method manufactures purified double-stranded nucleic acid that achieves a standard for purity, and wherein the standard for purity comprises the retentate comprising at least 80, 85, 90, 91, 92, 93, 94, 95, 96, 97, 98, or 99% double-stranded nucleic acid.
59. The method of any of the preceding clauses, wherein the method manufactures purified double-stranded nucleic acid that achieves a standard for purity, and wherein the standard for purity comprises the retentate comprising 80-100, 85-100, 90-100, 91-100, 92-100, 93-100, 94-100, 95-100, 96-100, 97-100, 98-100, 99-100, 80-99, 85-99, 90-99, 91-99, 92-99, 93-99, 94-99, 95-99, 96-99, 97-99, or 98-99% double-stranded nucleic acid.
60. The method of any of the preceding clauses, wherein the method manufactures purified double-stranded nucleic acid that achieves a standard for purity, and wherein the standard for purity comprises the retentate comprising 1-20, 1-15, 1-10, 1-9, 1-8, 1-7, 1-6, 1-5, 1-4, 1-3, 1-2, 1-1, 2-1, 3-1, 4-1, 5-1, 6-1, 7-1, 8-1, 9-1, 10-1, 15-1, or 20-1 of single-stranded nucleic acid to double-stranded nucleic acid.
61. The method of any of the preceding clauses, wherein the method achieves the standard for purity in no more than 25, 24, 23, 22, 21, or 20 DTVs.
62. The method or apparatus of any of the preceding clauses, wherein the length of at least one strand of the double-stranded nucleic acid and the length of the single-stranded nucleic acid are the same.
63. The method or apparatus of any of the preceding clauses, wherein the single-stranded nucleic acid is a component of the double-stranded nucleic acid.
64. The method or apparatus of any of the preceding clauses, wherein the double-stranded nucleic acid comprises a blunt end.
65. The method or apparatus of any of the preceding clauses, wherein the double-stranded nucleic acid comprises two blunt ends.
66. The method or apparatus of any of the preceding clauses, wherein the double-stranded nucleic acid comprises a single-stranded portion.
67. The method or apparatus of any of the preceding clauses, wherein the double-stranded nucleic acid has a single-stranded portion.
68. The method or apparatus of any of the preceding clauses, wherein the double-stranded nucleic acid comprises a conjugate group.
69. The apparatus of any of the preceding clauses, wherein the second chamber comprises permeate.
70. The apparatus of any of the preceding clauses, wherein the second chamber retains the permeate.
71. The apparatus of any of clauses 6-69, wherein the second chamber does not retain permeate.
72. The apparatus of any of the preceding clauses, wherein the first chamber comprises the mixture.
73. The apparatus of any of the preceding clauses, wherein the first chamber comprises the retentate.
74. The method or apparatus of any of the preceding clauses, wherein the mixture comprises no more than 1, 2, 3, 4, 5, 6, 7, 8, 9, 10, 20, 30, 40, 50, 60, 70, 80, 90, 100 μl, 200 μl, 300 μl, or 400 μl, or no more than 0.5, 1, 2, 3, 4, 5, 6, 7, 8, 9, 10, 20, 30, 40, 50, 60, 70, 80, 90, 100 ml, 200 ml, 300 ml, or 400 ml, or no more than 0.5, 1, 2, 3, 4, 5, 6, 7, 8, 9, 10, 20, 30, 40, 50, 60, 70, 80, 90, 100, 150, 200, 250, 300, 350, 400, 450, or 500 L, and optionally at least 1, 2, 3, 4, 5, 6, 7, 8, 9, 10, 20, 30, 40, 50, 60, 70, 80, 90, or 100 μl, or at least 0.5, 1, 2, 3, 4, 5, 6, 7, 8, 9, 10, 20, 30, 40, 50, 60, 70, 80, 90, or 100 ml, or at least 0.5, 1, 2, 3, 4, 5, 6, 7, 8, 9, 10, 20, 30, 40, 50, 60, 70, 80, 90, 100, 150, 200, 250, 300, 350, 400, 450, 500, 600, 700, 800, 900, or 1000 L.
75. The method or apparatus of any of the preceding clauses, wherein the mixture comprises at least 1, 2, 3, 4, 5, 6, 7, 8, 9, 10, 20, 30, 40, 50, 60, 70, 80, 90, 100 μl, 200 μl, 300 μl, or 400 μl, or at least 0.5, 1, 2, 3, 4, 5, 6, 7, 8, 9, 10, 20, 30, 40, 50, 60, 70, 80, 90, 100 ml, 200 ml, 300 ml, or 400 ml, or at least 0.5, 1, 2, 3, 4, 5, 6, 7, 8, 9, 10, 20, 30, 40, 50, 60, 70, 80, 90, 100, 150, 200, 250, 300, 350, 400, 450, or 500 L, and optionally no more than 1, 2, 3, 4, 5, 6, 7, 8, 9, 10, 20, 30, 40, 50, 60, 70, 80, 90, or 100 μl, or no more than 0.5, 1, 2, 3, 4, 5, 6, 7, 8, 9, 10, 20, 30, 40, 50, 60, 70, 80, 90, or 100 ml, or no more than 0.5, 1, 2, 3, 4, 5, 6, 7, 8, 9, 10, 20, 30, 40, 50, 60, 70, 80, 90, 100, 150, 200, 250, 300, 350, 400, 450, 500, 600, 700, 800, 900, or 1000 L.
76. The method or apparatus of any of the preceding clauses, wherein the mixture comprises:
1-100, 5-100, 10-100, 20-100, 30-100, 40-100, 50-100, 60-100, 70-100, 80-100, 90-100, 1-50, 5-50, 10-50, 20-50, 30-50, 40-50, 1-20, 5-20, 10-20, or 1-10 μl,
0.1-100, 0.5-100, 1-100, 5-100, 10-100, 20-100, 30-100, 40-100, 50-100, 60-100, 70-100, 80-100, 90-100, 0.1-50, 0.5-50, 1-50, 5-50, 10-50, 20-50, 30-50, 40-50, 0.1-20, 0.5-20, 1-20, 5-20, 10-20, 0.1-10, 0.5-10, 1-10, 0.1-1, 0.5-1, or 0.1-0.5 ml, or
0.1-500, 0.5-500, 1-500, 5-500, 10-500, 20-500, 30-500, 40-500, 50-500, 60-500, 70-500, 80-500, 90-500, 100-500, 150-500, 200-500, 250-500, 300-500, 350-500, 400-500, 450-500, 0.1-250, 0.5-250, 1-250, 5-250, 10-250, 20-250, 30-250, 40-250, 50-250, 60-250, 70-250, 80-250, 90-250, 100-250, 150-250, 200-250, 0.1-100, 0.5-100, 1-100, 5-100, 10-100, 20-100, 30-100, 40-100, 50-100, 60-100, 70-100, 80-100, 90-100, 0.1-50, 0.5-50, 1-50, 5-50, 10-50, 20-50, 30-50, 40-50, 0.1-20, 0.5-20, 1-20, 5-20, 10-20, 0.1-10, 0.5-10, 1-10, 0.1-1, 0.5-1, or 0.1-0.5 L.
77. The method or apparatus of any of the preceding clauses, wherein the mixture comprises a nucleic acid concentration of at least 100, 200, 300, 400, 500, 600, 700, 800, 900, 1000, 1100, 1200, 1300, 1400, 1500, 1600, 1700, 1800, 1900, 2000, 3000, 4000, 5000, 6000, 7000, 8000, or 9000 OD/ml.
78. The method or apparatus of any of the preceding clauses, wherein the mixture comprises a nucleic acid concentration of at least about 900 OD/mL.
79. The method or apparatus of any of the preceding clauses, wherein the mixture comprises a nucleic acid concentration of at least about 1100 OD/mL.
80. The method or apparatus of any of the preceding clauses, wherein the mixture comprises a nucleic acid concentration of at least about 400 OD/mL.
81. The method or apparatus of any of the preceding clauses, wherein the ssDNA in the permeate is composed primarily of sense strands.
82. The method or apparatus of any of the preceding clauses, wherein the ssDNA in the permeate is composed primarily of antisense strands.
83. The method or apparatus of any of the preceding clauses, wherein the double-stranded nucleic acid comprises a mismatch.
84. The method or apparatus of any of the preceding clauses, wherein the mixture comprises an amount of the single-stranded nucleic acid in excess of the amount of the double-stranded nucleic acid.
85. The method or apparatus of clause 84, wherein the amount of the single-stranded nucleic acid is in excess relative to the amount of the double-stranded nucleic acid by at least about 1%, 2%, 3%, 4%, 5%, 6%, 7%, 8%, 9%, 10%, 15%, 20%, 25%, 30%, 40%, 50%, 60%, 70%, 80%, or 90%.
86. The method or apparatus of clause 84, wherein the amount of the single-stranded nucleic acid is in excess relative to the amount of the double-stranded nucleic acid by about 5%.
87. The method of any of the preceding clauses, wherein the ultra-filtration step comprises diafiltration.
88. The method of clause 87, wherein the diafiltration is performed for at least 10, 20, 30, 40, or 50 minutes, or for at least 1, 2, 3, 4, 5, 6, 7, 8, 9, 10, 11, 12, 13, 14, 15, 16, 17, 18, 19, 20, 21, 22, 23, or 24 hours.
89. The method of clauses 87 or 88, wherein the diafiltration is performed a period of sufficient to filter at least about 2, 3, 4, 5, 6, 7, 8, 9, 10, 11, 12, 13, 14, 15, 20, 25, 27, 30, 35, 40, 45, 50, 55, 60, 63,65, 70, 75, 80, 81, 85, or 90 diafiltration volumes (DTVs).
90. The method of any of the preceding clauses, wherein the ultra-filtration is applied at a pressure of about 5-40, 10-40, 15-40, 20-40, 30-40, 35-40, 5-15, 5-20, 5-25, 5-30, 5-35, 10-30, 15-25, 5-10, 10-15, 15-20, 20-25, 25-30, 30-35, or 35-40 psi.
91. The method of any of the preceding clauses, wherein the ultra-filtration is applied at a pressure of about 15-30 psi.
92. The method of any of the preceding clauses, wherein the ultra-filtration is performed at a temperature of about 10-40° C., optionally wherein the ultra-filtration is performed at a temperature that is at least 0.5° C. below the melting point of the double-stranded nucleic acid.
93. The method of any of the preceding clauses, further comprising analyzing the retentate by non-denaturing HPLC.
94. The method of any of the preceding clauses, wherein the ratio of double-stranded nucleic acid relative to single-stranded nucleic acid in the retentate increases over time and/or over diafiltration total volume (DTV).
95. The method of any of the preceding clauses, wherein the ratio of double-stranded nucleic acid relative to single-stranded nucleic acid in the retentate is at least 90:5, 90.3:5, 90.4:4.8, 90.9:4.8, 91.1:4.6, 91.2:4.5, 91.1:4.4, 91.2:4.4, 91.3:4.1, or 91.5:3.9.
96. The method of any of the preceding clauses, wherein the ratio of double-stranded nucleic acid relative to single-stranded nucleic acid in the retentate is at least 90.1:5.3, 91.1:4.7, 91.2:4.8, 91.3:4.9, 92.0:3.5, 92.1:3.4, or 92.0:3.2.
97. The method or apparatus of any of the preceding clauses, wherein the concentration of a salt in the mixture is less than about 10, 20, 30, 40, 50, 60, 70, 80, 90, 100, 200, 300, 400, 500, 600, 700, 800, 900, 1000, 1500, 2000, or 3000 mM.
98. The method or apparatus of clause 97, wherein the mixture does not substantially comprise the salt.
99. The method or apparatus of any of the preceding clauses, wherein the concentration of a salt in the mixture is about 50 mM to about 3M.
100. The method or apparatus of any of the preceding clauses, wherein the melting temperature of the double-stranded nucleic acid in the mixture is between about 20-85° C.
101. The method or apparatus of any of the preceding clauses, wherein the relationship between the melting temperature of the double-stranded nucleic acid in the mixture and the concentration of a salt in the mixture is determined.
102. The method of any of the preceding clauses, wherein the double-stranded nucleic acid of the retentate is for use in in a downstream use or application.
103. The method of any of the preceding clauses, wherein the double-stranded nucleic acid of the retentate is lyophilized.
104. The method of any of the preceding clauses, wherein the double-stranded nucleic acid of the retentate is conjugated with an additional agent.
105. The method of any of the preceding clauses, wherein the double-stranded nucleic acid of the retentate is mixed with other oligonucleotide duplexes.
106. The method of any of the preceding clauses, wherein the double-stranded nucleic acid of the retentate is mixed with a drug product formulation buffer.
107. The method or apparatus of any of the preceding clauses, wherein the double-stranded nucleic acid has a length of 5-10, 10-15, 15-20, 20-25, 25-30, 30-40, 40-50, 50-60, 60-70, 70-80, 80-90, 90-100, 100-125, 125-150, 150-200, 200-250, 250-300, 300-400, or 400-500 nucleotides.
108. The method of any of the preceding clauses, wherein the method reduces the level of low-molecular weight impurities by at least 25, 50, 75, 80, 85, 90, 95, 96, 97, 98, 99, or 100%.
109. The method of clause 108, wherein the low-molecular weight impurities each have a molecular weight of less than about 500, 400, 300, 200, 100, 50, 40, 30, 20, or 10 Daltons.
110. The method of clause 109, wherein the low-molecular weight impurities comprise salts and/or trace solvents.
111. The method of any of the preceding clauses, wherein the method reduces the level of the double-stranded nucleic acid by no more than 0.1, 0.5, 1, 2, 3, 4, 5, 6, 7, 8, 9, or 10%.
112. The method of any of the preceding clauses, wherein the ratio of the amount of the double-stranded nucleic acid in the retentate and the amount of the double-stranded nucleic acid in the mixture is at least 0.75, 0.8, 0.85, 0.9, 0.95, 0.96, 0.97, 0.98, 0.99, 0.995, or 0.999.
113. The method of any of the preceding clauses, wherein the quantity of the double-stranded nucleic acid in the retentate is at least 20, 22, 24, 26, 28, 30, 32, 34, 36, 38, 40, 42, 44, 46, 48, or 50 mg/mL.
114. A method of optimizing a technique for manufacturing a purified double-stranded nucleic acid, comprising:
   (i) providing a mixture comprising:
      a double-stranded nucleic acid comprising a first single-stranded nucleic acid and a second single-stranded nucleic acid at a first concentration, and
      a single-stranded nucleic acid;
      wherein the mixture has a predetermined concentration of a salt;
   (ii) subjecting the mixture to an ultra-filtration step that selectively retains double-stranded nucleic acid comprising the first and second single-stranded nucleic acids and not the single-stranded nucleic acid,
      wherein the ultra-filtration step comprises applying the mixture to a filtration apparatus with a predetermined molecular weight cutoff for a predetermined length of time
   (iii) harvesting the retentate;
   (iv) varying one of or more of the first concentration, the second concentration, the predetermined molecular weight cutoff, the predetermined length of time, or the salt concentration;
   (v) repeating steps (i)-(iii) one or more times, each using one or more of the varied first concentration, second concentration, predetermined molecular weight cutoff, predetermined length of time, and/or salt concentration;
   (vi) determining, for each repetition of steps (i)-(iii), the ratio of double-stranded nucleic acid to single-stranded nucleic acid in the harvested retentates; and
   (vii) identifying a first concentration, the second concentration, predetermined molecular weight cutoff, predetermined length of time, and/or salt concentration that yields the highest ratio of double-stranded nucleic acid to single-stranded nucleic acid in the harvested retentates;
thereby optimizing a technique for manufacturing a purified double-stranded nucleic acid.
115. A double-stranded nucleic acid composition comprising a harvested retentate produced according to the method of any of clauses 1 or 7-113.
116. A single-stranded nucleic acid composition comprising a harvested permeate produced according to the method of any of clauses 2 or 7-113.
117. A composition comprising a first double-stranded nucleic acid comprising a first overhang hybridized to a second double-stranded nucleic acid comprising a second overhang.
118. The composition of clause 117, wherein the first double-stranded nucleic acid is made according to the method of any of the preceding claims.
119. The composition of clause 117 or 118, wherein the second double-stranded nucleic acid is made according to the method of any of the preceding claims.
120. A method of producing a circular double-stranded nucleic acid, comprising:
   (a) providing the composition of any of claims 117-119, and
   (b) incubating the composition under conditions suitable for circularization of the first double-stranded nucleic acid hybridized to the second double-stranded nucleic acid.
121. The method of clause 120, wherein the circularization comprises ligation.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 depicts a schematic representation of an exemplary ultrafiltration apparatus for use in the methods described herein. Duplex oligonucleotide solution, that may contain excess non-hybridized single strands, is loaded into the ultrafiltration system's retentate tank. The material will be pumped through the ultrafiltration membrane and back into the retentate tank. Pressure will be applied to the flow path which in turn mechanically forces (permeates) compounds with a molecular weight below the molecular weight cut off of the ultrafiltration membrane to the permeate side of the membrane. Process water is added to the retentate tank to maintain a proper working volume while the low molecular weight species and water permeate through the ultrafiltration membrane. The process is monitored by conductivity of the permeate stream. A conductivity threshold target is set to facilitate the end point for clearance of ionic low molecular weight species to an acceptable level.

FIGS. 2A-2B are images of a UV chromatograph representing reverse phase chromatographs of diafiltration time-point studies for a composition of duplex and non-hybridized single-stranded nucleic acid over time. FIG. 2A represents the full HPLC run time. FIGS. 2B and 2C represent the time difference between single stranded and duplex nucleic acids (left peak and right peak, respectively). This study was performed as described in Example 3.

### DETAILED DESCRIPTION

The present disclosure is directed, at least in part, to improved methods of producing (e.g., purifying) oligonucleotides (e.g., separating double-stranded oligonucleotides from single-stranded oligonucleotides). Without wishing to be bound by theory, it is believed that in some embodiments methods of producing (e.g., purifying) oligonucleotides, particularly in a large scale or industrial manufacturing setting or using modified, synthetic, or non-natural nucleotides, are generally highly sensitive to process parameters such as the concentrations of agents in solutions, the ratios of agents in solutions to one another, the duration of a step (e.g., recycling time), membrane pressure, membrane porosity or composition, or the presence and/or level of a contaminant in a production solution. Accordingly, the present disclosure is based, at least in part, on the discovery that changes in production (e.g., purification) parameters can alter the efficacy of the method, e.g., by altering one or more of the amount of oligonucleotide purified, a product characteristic of the oligonucleotide (e.g., length, purity, composition (e.g., the correct nucleotides, e.g., modified nucleotides), sequence, or annealed state), or the amount of a resource or time consumed.

### I. Definitions

The term “about”, as used herein, refers to the usual error range for the respective value readily known to the skilled person in this technical field. As such, the term “about” may be used to indicate that a value includes the inherent variation of error for the device or the method being employed to determine the value. For example, the term “about” generally means ±10% (e.g., ±1, 2, 3, 4, 5,6, 7, 8, 9, 10%) of the stated amount.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

The term “cross-flow filter,” as used herein, refers to a filter in which the feed is passed tangentially across the surface of the filter. In some embodiments, the cross-flow filter includes a transmembrane pressure.

The term “diafiltration tank volume (DTV),” as used herein, refers to a working volume in which a substance of interest (e.g., a double-stranded nucleic acid or a single-stranded nucleic acid) is suspended and maintained through the process, e.g., prior to a purification step (e.g., comprising ultrafiltration) as described herein. In some embodiments, DTV is a measure of the volume that has been flowed through during a diafiltration step based on the volume introduced into the unit operation compared to the retentate volume. For example, if a volume equal to the working volume contained in the retentate tank has been flowed through, then the diafiltration volume is equal to 1; if a volume equal to twice the entire initial volume has been flowed through, then the diafiltration volume is equal to 2; and so on.

The term “molecular weight cutoff” (MWCO), as used herein, refers to a molecular weight target that is reflective of the average pore size. In some embodiments, substances that exceed the MWCO (e.g., a double-stranded nucleic acid, single stranded nucleic acid, or other mixture component) are substantially retained by a membrane (e.g., of which at least 75%, 80%, 85%, 90%, 95%, 96%, 97%, 98%, 99%, or 100% of the original substance is retained), e.g., in an ultrafiltration method as described herein. In some embodiments, the MWCO is at least about 1, 2, 3, 4, 5, 6, 7, 8, 9, or 10 kilodaltons less than the MW of the double-stranded nucleic acid. In some embodiments, the MWCO is no more than about 20, 15, 10, 9, 8, 7, 6, 5, 4, 3, or 2 kilodaltons less than the MW of the double stranded nucleic acid. In some embodiments, a substance species can transit a membrane in a molecular weight distribution or range with as much as +/−1 kDa. By way of example, a 10 kDa MWCO may permeate a substance species having a molecule weight of 10+/−1 kDa.

The term “pore size,” as used herein, refers to the average diameter of a pore in a barrier (e.g., a membrane, e.g., a semi-permeable membrane), e.g., for filtering out particles of a certain size, e.g., by way of ultrafiltration. In some embodiments, the pore size has an average diameter of at least about 1, 1.2, 1.4, 1.6, 1.8, 2, 2.2, 2.4, 2.6, 2.8, or 3 nm. In some embodiments, the pore size has an average diameter of no more than about 0.001, 0.005, 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1.0, 1.05, 1.1, 1.5, or 1.6 μm. Additionally, in other embodiments the pore size is presented as a MWCO of 1, 2, 3, 5, 10, 20, 30, 50, 100 kDa.

As used herein, in its broadest sense, the term “nucleic acid” refers to any compound and/or substance that is or can be incorporated into an oligonucleotide chain. In some embodiments, a nucleic acid is a compound and/or substance that is or can be incorporated into an oligonucleotide chain via a phosphodiester linkage. In some embodiments, “nucleic acid” refers to an individual nucleic acid monomer (e.g., a nucleotide and/or nucleoside); in some embodiments, “nucleic acid” refers to an oligonucleotide chain comprising individual nucleic acid monomers. In some embodiments, a “nucleic acid” is or comprises RNA. In some embodiments, a “nucleic acid” is or comprises DNA. In some embodiments, a nucleic acid is, comprises, or consists of one or more natural nucleic acid residues. In some embodiments, a nucleic acid is, comprises, or consists of one or more nucleic acid analogs. In some embodiments, a nucleic acid is, comprises, or consists of one or more modified, synthetic, or non-naturally occurring nucleotides. In some embodiments, a nucleic acid analog differs from a nucleic acid in that it does not utilize a phosphodiester backbone. For example, in some embodiments, a nucleic acid is, comprises, or consists of one or more “peptide nucleic acids,” which are known in the art and have peptide bonds instead of phosphodiester bonds in the backbone, are considered within the scope of the present invention. Alternatively or additionally, in some embodiments, a nucleic acid has one or more phosphorothioate and/or 5′-N-phosphoramidite linkages rather than phosphodiester bonds. In some embodiments, a nucleic acid is, comprises, or consists of one or more natural nucleosides (e.g., adenosine, thymidine, guanosine, cytidine, uridine, deoxyadenosine, deoxythymidine, deoxy guanosine, and deoxycytidine). In some embodiments, a nucleic acid is, comprises, or consists of one or more nucleoside analogs (e.g., 2′methoxy ethyl (2′MOE), 2′fluoro, 2′OMe, 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, 2-thiocytidine, methylated bases, intercalated bases, and combinations thereof). In some embodiments, a nucleic acid comprises one or more modified sugars (e.g., 2′-fluororibose, ribose, 2′-deoxyribose, arabinose, and hexose) as compared with those in natural nucleic acids. In some embodiments, a nucleic acid comprises an N-acetylgalactosamine (GalNAc) modification, e.g., to the sugar, phosphate, phosphodiester, or base of a nucleic acid, e.g., to the phosphodiester. In some embodiments, a nucleic acid has a nucleotide sequence that encodes a functional gene product such as an RNA or protein. In some embodiments, a nucleic acid is a noncoding nucleic acid (e.g., a primer (e.g., a DNA primer) or a noncoding RNA (e.g., a functional RNA)). In some embodiments, a nucleic acid includes one or more introns. In some embodiments, a nucleic acid is partly or wholly single stranded. In some embodiments, a nucleic acid is partly or wholly double stranded. In some embodiments, a nucleic acid has a nucleotide sequence comprising at least one element that encodes, or is the complement of a sequence that encodes, a polypeptide, or a portion thereof. In some embodiments, a nucleic acid has enzymatic activity (e.g., the nucleic acid is a ribozyme).

As used herein, an “oligonucleotide” refers to a nucleic acid that is at least 2 nucleotides long and no more than about 500 nucleotides long. In some embodiments, an oligonucleotide is 5-10, 10-15, 15-20, 20-25, 25-30, 30-40, 40-50, 50-60, 60-70, 70-80, 80-90, 90-100, 100-125, 125-150, 150-175, 175-200, 10-150, or 14-100 nucleotides long. Oligonucleotides may be useful in a variety of biological contexts, including but not limited to the manufacture and use of therapeutics, laboratory applications (e.g., PCR, RNAi, genetic modification (e.g., CRISPR genetic editing), sequencing), and biological manufacturing.

The term “other mixture components,” as used herein, refers to components in a mixture that are not a molecule of interest (e.g., a double-stranded nucleic acid or a single-stranded nucleic acid). In some embodiments, the other mixture components include a salt (e.g., a halide or mineral salt). In some embodiments, the other mixture components include a buffering agent. In certain embodiments, the other mixture components include a production reagent or a byproduct (e.g., a truncated nucleic acid sequence or a non-desired nucleic acid sequence). In some embodiments, the other mixture components include a shorter length single-stranded nucleic acid (e.g., shorter than a first and/or second single-stranded nucleic acid of interest). In some embodiments, the other mixture components include an organic solvent (e.g., acetonitrile, ethanol, dimethylformamide, DMSO, toluene, pyridine, or lutidine). In some embodiments, the other mixture components include a thiolation reagent/by-product (e.g., xanthane, hydride, or PADS) or a capping reagent (e.g., acetic anhydride or NMI). In some embodiments, the other mixture components include a coupling reagent (e.g., phosporamidite or ETT). In some embodiments, the other mixture components include a detritylation reagent/by-product (e.g., dichloroacetic acid). In some embodiments, the other mixture components include a deprotection reagent/by-product (e.g., diethylamine, methylamine, or ammonia). In some embodiments, a production reagent or byproduct may be selected from a shorter length single strand nucleic acid (e.g., shorter than the first and/or second single-stranded nucleic acid), an organic solvent (e.g., acetonitrile, ethanol, dimethylformamide, DMSO, toluene, pyridine, or lutidine), a thiolation reagent (e.g., xanthane, hydride, or PADS), a capping reagent (e.g., acetic anhydride or NMI), a coupling reagent (e.g., phosporamidite or ETT), a detritylation reagent (e.g., dichloroacetic acid), a conjugation reagent (e.g., carboxylic acid, carboxylic ester, carbonate, and carboxylic acid activator reagents), and/or a deprotection reagent (e.g., diethylamine, methylamine, or ammonia).

The term “permeate,” as used herein, refers to the substances (e.g., single-stranded nucleic acids) that pass through a barrier (e.g., membrane, e.g., semi-permeable membrane)), e.g., in an ultrafiltration method as described herein. In some embodiments, the permeate comprises single-stranded nucleic acids. In some embodiments, the permeate comprises other mixture components. In some embodiments, the permeate comprises little or no detectable levels of double-stranded nucleic acids. In some embodiments, the stoichiometric ratio of single-stranded nucleic acid to double-stranded nucleic acids in the retentate is at least 100:1, 1,000:1, 10,000:1, 100,000:1, 1,000,000:1, or more.

As used herein, a “phosphoramidite nucleotide” refers to a nucleotide comprising a phosphate group that comprises a monoamide phosphite diester. In some embodiments, a phosphoramidite nucleotide is useful for a nucleotide addition step described herein. Without wishing to be bound by theory, a phosphoramidite nucleotide is thought to be reactive in the presence of a weak acid and a nucleophile (e.g., a nucleic acid (e.g., 3′ OH) of loaded solid media).

The term “post-synthesis step,” as used herein, generally refers to a step of a method that is performed after a synthesis phase of the method. In some embodiments, the post-synthesis step includes chemical or enzymatic cleavage from a solid substrate. In some embodiments, the post-synthesis step includes removal of one or more chemical moieties (e.g., removal of a protecting group). In some embodiments, the post-synthesis step includes chemical or enzymatic cleavage from a solid substrate and/or removal of one or more chemical moieties (e.g., removal of a protecting group). In some embodiments the post-synthesis step includes chemical conjugation with conjugation reagents (e.g., carboxylic acid, carboxylic ester, carbonate, and carboxylic acid activator reagents).

The term “retentate,” as used herein, refers to the substances (e.g., double-stranded nucleic acids) that do not pass through a barrier (e.g., membrane, e.g., semi-permeable membrane, e.g., filter), e.g., in an ultrafiltration method as described herein. In some embodiments, the retentate comprises double-stranded nucleic acids. In some embodiments, the permeate comprises other mixture components. In some embodiments, the retentate comprises little or no detectable levels of single-stranded nucleic acids. In some embodiments, the stoichiometric ratio of double-stranded nucleic acid to single-stranded nucleic acids in the retentate is at least 100:1, 1,000:1, 10,000:1, 100,000:1, 1,000,000:1, or more.

The term “selectively retains,” as used herein, refers to the retention of desired materials, e.g., in the retentate, e.g., by a method and/or in a system or apparatus as described herein. In some embodiments, the methods, apparatuses, and systems described herein selectively retain double-stranded nucleic acids.

The term, “single-stranded form,” as used herein, refers to a compound comprising a nucleic acid or oligonucleotide that is substantially (e.g., entirely) single-stranded, e.g., is not interacting (e.g., hybridizing) with another nucleic acid or oligonucleotide (e.g., another nucleic acid that is sufficiently complementary or a second portion of the nucleic acid that is sufficiently complementary to a first portion of the nucleic acid).

The term “sufficiently complementary,” as used herein, refers to a first nucleic acid sequence comprising a contiguous nucleic acid sequence that permits a second nucleic acid (e.g., comprising a nucleic acid sequence having at least 75%, 80%, 85%, 90%, 95%, 96%, 97%, 98%, 99%, or 100% sequence identity to the reverse complement of the contiguous nucleic acid sequence of the first nucleic acid) to hybridize thereto. Sufficiently complementary sequences can include Watson-Crick base pairs formed from natural and/or modified nucleic acids. Sufficiently complementary sequences can also include non-Watson-Crick base pairs, such as wobble base pairs (guanosine-uracil, hypoxanthine-uracil, hypoxanthine-adenine, and hypoxanthine-cytosine) and Hoogsteen base pairs. In some embodiments, the nucleic acid sequence from a first single-stranded nucleic acid is sufficiently complementary to hybridize with a nucleic acid sequence from a second single-stranded nucleic acid to form a double-stranded nucleic acid. In some embodiments, the nucleic acid from a second single-stranded nucleic acid is sufficiently complementary to hybridize with a nucleic acid sequence from a first single-stranded nucleic acid to form a double-stranded nucleic acid. In some embodiments, a first nucleic acid sequence from a first nucleic acid is sufficiently complementary to hybridize with a second nucleic acid sequence from the first nucleic acid, e.g., forming a hairpin loop. In some embodiments, a first nucleic acid sequence from a second nucleic acid is sufficiently complementary to hybridize with a second nucleic acid sequence from the second nucleic acid, e.g., forming a hairpin loop. In some embodiments, a first nucleic acid sequence from a first nucleic acid is not sufficiently complementary to hybridize with a second nucleic acid sequence from the first nucleic acid, e.g., forming a hairpin loop. In some embodiments, a first nucleic acid sequence from a second nucleic acid is not sufficiently complementary to hybridize with a second nucleic acid sequence from the second nucleic acid, e.g., forming a hairpin loop.

The term “synthesis phase,” as used herein, generally refers to a step or a plurality of steps of a method that produce a product or effect one or more additions to a substrate. In some embodiments, a synthesis phase comprises one or more “synthesis steps” of a method as described herein, e.g., wherein the steps add one or more nucleotide monomers to a nucleic acid of loaded solid media. In some embodiments, one or more steps of a synthesis phase may be repeated, e.g., to sequentially add one or more nucleotide monomers to a nucleic acid of loaded solid media. In some embodiments, a method of making an oligonucleotide comprises additional phases or steps besides synthesis phase or the steps contained in synthesis phase. In some embodiments, the synthesis step includes the solid-state chemical synthesis of a first single-stranded nucleic acid, a second single-stranded nucleic acid, or both. In some embodiments, oligonucleotide synthesis pertains to the creation of an oligonucleotide via polymerase chain reaction or generation via a cellular organism.

The term, “transmembrane pressure,” as used herein, refers to a hydrostatic pressure gradient which allows for ultrafiltration or convection across a barrier (e.g., a membrane, e.g., a semi-permeable membrane, e.g., as described herein). In some embodiments, the transmembrane pressure is at least about 5, 6, 7, 8, 9, 10, 11, 12, 13, 14, 15, 16, 17, 18, 19, 20, 21, 22, 23, 24, 25, 26, 27, 28, or 29 psi. In some embodiments, the transmembrane pressure is no more than about 30, 29, 28, 27, 26, 25, 24, 23, 22, 21, 20, 19, 18, 17, 16, 15, 14, 13, 12, 11, 10, 9, 8, 7, or 6 psi.

The term “ultra-filtration,” as used herein, refers to a filtration step comprising applying a solution to a pressure-driven barrier (e.g., a membrane, e.g., a semi-permeable membrane)). Solutes of high molecular weight are retained, while water and low molecular weight solutes pass through the barrier (e.g., the membrane, e.g., the semi-permeable membrane). In some embodiments, ultra-filtration comprises diafiltration. In certain embodiments, ultra-filtration comprises tangential flow filtration. In some embodiments, ultra-filtration comprises diafiltration or tangential flow filtration.

### II. Methods of Production

The disclosure is directed in part to methods of producing (e.g., manufacturing) purified oligonucleotides (e.g., purified single-stranded oligonucleotides and/or purified double-stranded oligonucleotides). Generally, a method of producing (e.g., manufacturing) purified oligonucleotides of the present disclosure comprises one or more steps that purify a duplexed (e.g., double-stranded) oligonucleotide from a mixture (e.g., a mixture comprising double-stranded oligonucleotides and single-stranded oligonucleotides). In certain embodiments, the method comprises, or is, a manufacturing method, e.g., a large-scale manufacturing method (e.g., is not a research-scale method or is not an analytical method). In some embodiments, a method of producing (e.g., manufacturing) purified oligonucleotides of the present disclosure comprises one or more steps that purify an oligonucleotide in double-stranded form (e.g., double-stranded oligonucleotides). In some embodiments, a method of producing (e.g., manufacturing) purified oligonucleotides of the present disclosure comprises one or more steps that purify an oligonucleotide in single-stranded form (e.g., single-stranded oligonucleotides). A method of producing (e.g., manufacturing) a purified oligonucleotide (e.g., a purified single-stranded nucleic acid or a purified double-stranded oligonucleotide) can include, for example, providing a mixture, subjecting the mixture to an ultrafiltration step, and harvesting the retentate (e.g., retentate comprising the double-stranded oligonucleotide). A method of producing (e.g., manufacturing) a purified oligonucleotide (e.g., a purified single-stranded nucleic acid or double-stranded oligonucleotide) can include, for example, providing a mixture, subjecting the mixture to an ultrafiltration step, and harvesting the permeate.

In some embodiments, providing a mixture comprises: providing a first single-stranded nucleic acid and a second single-stranded nucleic acid which comprise sequences that are sufficiently complementary to one another to hybridize under conditions suitable for hybridization, e.g., as described in Examples 1-5, and combining the first single-stranded nucleic acid and second single-stranded nucleic acid under conditions suitable for hybridization to produce a mixture comprising: a double-stranded nucleic acid comprising the first single-stranded nucleic acid and the second single-stranded nucleic acid, and the first single-stranded nucleic acid in single-stranded form, the second single-stranded nucleic acid in single-stranded form, or the first single-stranded nucleic acid in single-stranded form and the second single-stranded nucleic acid in single-stranded form.

In some embodiments, providing a first single-stranded nucleic and a second single-stranded nucleic acid comprises a synthesis step. In some embodiments, the synthesis step includes solid-state chemical synthesis of the first single-stranded nucleic acid, the second single-stranded nucleic acid, or the first single-stranded nucleic acid and the second single-stranded nucleic acid.

In some embodiments, providing a first single-stranded nucleic acid and a second single-stranded nucleic acid comprises a post synthesis step. In some embodiments, the post synthesis step includes a chemical or enzymatic cleavage from a solid substrate, removal of one or more chemical moieties (e.g., removal of a protecting group), or a chemical or enzymatic cleavage from a solid substrate and removal of one or more chemical moieties (e.g., removal of a protecting group). In some embodiments the post-synthesis step includes chemical conjugation with conjugation reagents (e.g., carboxylic acid, carboxylic ester, carbonate, and carboxylic acid activator reagents).

In some embodiments, providing a first single-stranded nucleic acid and a second single-stranded nucleic acid comprises a purification step. In some embodiments, the purification step is a step that enriches for nucleic acid components and decrease the level of non-nucleic acid components. In some embodiments, the purification step comprises a chromatography step, e.g., anion exchange chromatography.

In some embodiments, providing a first single-stranded nucleic acid and a second single-stranded nucleic acid includes a subjecting the first single-stranded nucleic acid, the second single-stranded nucleic acid, or the first single-stranded nucleic acid and the second single-stranded nucleic acid to an ultrafiltration step that selectively retains single-stranded nucleic acid. In some embodiments, the ultra-filtration step selectively retains single-stranded nucleic acid and not non-nucleic acid components.

In some embodiments, the first single-stranded nucleic acid is at least 10 nucleotides long, e.g., at least 11, 12, 13, 14, 15, 16, 17, 18, 19, 20, 21, 22, 23, 24, 25, 26, 27, 28, 29, or 30 nucleotides long. In some embodiments, the first single-stranded nucleic acid is no more than 100 nucleotides long, e.g., no more than 80, 60, 50, 45, 40, 35, or 30 nucleotides long. In some embodiments, the first single-stranded nucleic acid is at least 10, 11, 12, 13, 14, 15, 16, 17, 18, 19, 20, 21, 22, 23, 24, 25, 26, 27, 28, 29, or 30 nucleotides long and optionally is no more than 100, 80, 60, 50, 45, 40, 35, or 30 nucleotides long.

In some embodiments, the first single-stranded nucleic acid is 10-50 nucleotides long, e.g., 12-50, 14-50, 16-50, 18-50, 20-50, 22-50, 24-50, 26-50, 28-50, 30-50, 32-50, 34-50, 36-50, 38-50, 40-50, 42-50, 44-50, 46-50, 48-50, 10-45, 12-45, 14-45, 16-45, 18-45, 20-45, 22-45, 24-45, 26-45, 28-45, 30-45, 32-45, 34-45, 36-45, 38-45, 40-45, 42-45, 10-40, 12-40, 14-40, 16-40, 18-40, 20-40, 22-40, 24-40, 26-40, 28-40, 30-40, 32-40, 34-40, 36-40, 38-40, 10-35, 12-35, 14-35, 16-35, 18-35, 20-35, 22-35, 24-35, 26-35, 28-35, 30-35, 32-35, 10-30, 12-30, 14-30, 16-30, 18-30, 20-30, 22-30, 24-30, 26-30, 28-30, 10-25, 12-25, 14-25, 16-25, 18-25, 20-25, 22-25, 10-20, 12-20, 14-20, 16-20, 18-20, 10-15, or 12-15 nucleotides long.

In some embodiments, the first single-stranded nucleic acid is 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides long.

In some embodiments, the second single-stranded nucleic acid is at least 10 nucleotides long, e.g., at least 11, 12, 13, 14, 15, 16, 17, 18, 19, 20, 21, 22, 23, 24, 25, 26, 27, 28, 29, or 30 nucleotides long. In some embodiments, the second single-stranded nucleic acid is no more than 100 nucleotides long, e.g., no more than 80, 60, 50, 45, 40, 35, or 30 nucleotides long. In some embodiments, the second single-stranded nucleic acid is at least 10, 11, 12, 13, 14, 15, 16, 17, 18, 19, 20, 21, 22, 23, 24, 25, 26, 27, 28, 29, or 30 nucleotides long and optionally is no more than 100, 80, 60, 50, 45, 40, 35, or 30 nucleotides long.

In some embodiments, the second single-stranded nucleic acid is 10-50 nucleotides long, e.g., 12-50, 14-50, 16-50, 18-50, 20-50, 22-50, 24-50, 26-50, 28-50, 30-50, 32-50, 34-50, 36-50, 38-50, 40-50, 42-50, 44-50, 46-50, 48-50, 10-45, 12-45, 14-45, 16-45, 18-45, 20-45, 22-45, 24-45, 26-45, 28-45, 30-45, 32-45, 34-45, 36-45, 38-45, 40-45, 42-45, 10-40, 12-40, 14-40, 16-40, 18-40, 20-40, 22-40, 24-40, 26-40, 28-40, 30-40, 32-40, 34-40, 36-40, 38-40, 10-35, 12-35, 14-35, 16-35, 18-35, 20-35, 22-35, 24-35, 26-35, 28-35, 30-35, 32-35, 10-30, 12-30, 14-30, 16-30, 18-30, 20-30, 22-30, 24-30, 26-30, 28-30, 10-25, 12-25, 14-25, 16-25, 18-25, 20-25, 22-25, 10-20, 12-20, 14-20, 16-20, 18-20, 10-15, or 12-15 nucleotides long.

In some embodiments, the second single-stranded nucleic acid is 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides long.

In some embodiments, the double-stranded nucleic acid is at least 10base pairs long, e.g., at least 11, 12, 13, 14, 15, 16, 17, 18, 19, 20, 21, 22, 23, 24, 25, 26, 27, 28, 29, or 30 base pairs long. In some embodiments, the double-stranded nucleic acid is no more than 100 base pairs long, e.g., no more than 80, 60, 50, 45, 40, 35, or 30 base pairs long). In some embodiments, the double-stranded nucleic acid is at least 10, 11, 12, 13, 14, 15, 16, 17, 18, 19, 20, 21, 22, 23, 24, 25, 26, 27, 28, 29, or 30 base pairs long and optionally no more than 100, 80, 60, 50, 45, 40, 35, or 30 base pairs long.

In some embodiments, the double-stranded nucleic acid is 10-50 base pairs long, e.g., 12-50, 14-50, 16-50, 18-50, 20-50, 22-50, 24-50, 26-50, 28-50, 30-50, 32-50, 34-50, 36-50, 38-50, 40-50, 42-50, 44-50, 46-50, 48-50, 10-45, 12-45, 14-45, 16-45, 18-45, 20-45, 22-45, 24-45, 26-45, 28-45, 30-45, 32-45, 34-45, 36-45, 38-45, 40-45, 42-45, 10-40, 12-40, 14-40, 16-40, 18-40, 20-40, 22-40, 24-40, 26-40, 28-40, 30-40, 32-40, 34-40, 36-40, 38-40, 10-35, 12-35, 14-35, 16-35, 18-35, 20-35, 22-35, 24-35, 26-35, 28-35, 30-35, 32-35, 10-30, 12-30, 14-30, 16-30, 18-30, 20-30, 22-30, 24-30, 26-30, 28-30, 10-25, 12-25, 14-25, 16-25, 18-25, 20-25, 22-25, 10-20, 12-20, 14-20, 16-20, 18-20, 10-15, or 12-15 base pairs long.

In some embodiments, the double-stranded nucleic acid is 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides long.

In some embodiments, the double-stranded nucleic acid comprises a sequence having at least 30%, 40%, 50%, 60%, 70%, 75%, 80%, 85%, 90%, 95%, 96%, 97%, 98%, 99%, or 100% sequence identity to a nucleic acid sequence of interest (e.g., the sequence of a target gene, non-coding RNA, primer, or component for molecule assembly). In some embodiments, the double-stranded nucleic acid comprises a sequence differing by no more than 1, 2, 3, 4, 5, 6, 7, 8, 9, or 10 nucleotides from a nucleic acid sequence of interest (e.g., the sequence of a target gene, non-coding RNA, primer, or component for molecule assembly).

In some embodiments, the double-stranded nucleic acid comprises a sequence having at least 30%, 40%, 50%, 60%, 70%, 75%, 80%, 85%, 90%, 95%, 96%, 97%, 98%, 99%, or 100% sequence complementarity to a nucleic acid sequence of interest (e.g., the sequence of a target gene, non-coding RNA, primer, or component for molecule assembly). In some embodiments, the double-stranded nucleic acid comprises a sequence differing by no more than 1, 2, 3, 4, 5, 6, 7, 8, 9, or 10 nucleotides from a sequence complimentary to a nucleic acid sequence of interest (e.g., the sequence of a target gene, non-coding RNA, primer, or component for molecule assembly).

In some embodiments, the ultra-filtration step comprises diafiltration. Generally, diafiltration comprises separation of components in a solution (e.g., double-stranded nucleic acids, single-stranded nucleic acids, salts, solvents, or other molecules as described herein) based on their molecular size (e.g., molecular weight), e.g., using permeable filters. In some embodiments, the diafiltration comprises flowing a solution to be filtered through a permeable filter, e.g., as described herein. In some embodiments, the diafiltration comprises repeatedly filtering a solution through a permeable filter, e.g., as described herein, e.g., for a certain length of time. In some embodiments, the diafiltration is performed for at least 10, 20, 30, 40, or 50 minutes, or for at least 1, 2, 3, 4, 5, 6, 7, 8, 9, 10, 11, 12, 13, 14, 15, 16, 17, 18, 19, 20, 21, 22, 23, or 24 hours. In some embodiments, the diafiltration is performed a period of sufficient to filter at least about 2, 3, 4, 5, 6, 7, 8, 9, 10, 11, 12, 13, 14, 15, 20, 25, 27, 30, 35, 40, 45, 50, 55, 60, 63,65, 70, 75, 80, 81, 85, or 90 diafiltration volumes (DTVs). In some embodiments, the ultra-filtration is applied at a pressure of about 5-40, 10-40, 15-40, 20-40, 30-40, 35-40, 5-15, 5-20, 5-25, 5-30, 5-35, 10-30, 15-25, 5-10, 10-15, 15-20, 20-25, 25-30, 30-35, or 35-40 psi. In some embodiments, the ultra-filtration is applied at a pressure of about 15-30 psi (e.g., about 15-25, 15-20, 25-30, 20-30, 15-20, 20-25, or 25-30 psi, e.g., about 15, 16, 17, 18, 19, 20, 21, 22, 23, 24, 25, 26, 27, 28, 29, or 30 psi). In some embodiments, the ultra-filtration is performed at a temperature of about 10-40° C., e.g., about 15-40, 20-40, 25-40, 30-40, 35-40, 10-35, 10-30, 10-25, 10-20, 10-15, 15-35, 20-30, 10-15, 15-20, 20-25, 25-30, 30-35, or 35-40° C., e.g., about 10, 15, 20, 25, 30, 35, 37, or 40° C.

In some embodiments, the method of manufacture further includes analyzing the retentate by non-denaturing HPLC (e.g., to determine the relative quantity of double-stranded nucleic acid and/or single-stranded nucleic acid in the retentate), e.g., as described in Examples 1-5. In some embodiments, the ratio of double-stranded nucleic acid relative to single-stranded nucleic acid in the retentate (e.g., as determined by non-denaturing HPLC) increases over time and/or over diafiltration total volume (DTV). In some embodiments, the ratio of double-stranded nucleic acid relative to single-stranded nucleic acid in the retentate (e.g., as determined by non-denaturing HPLC) is at least 1:1, 2:1, 3:1, 4:1, 5:1, 6:1, 7:1, 8:1, 9:1, 10:1, 11:1, 12:1, 13:1, 14:1, 15:1, 20:1, 25:1, 30:1, 40:1, 50:1 60:1, 70:11 80:1, 90:1, 100:1, or 200:1.

In some embodiments, the method reduces the level of low-molecular weight impurities by at least 25, 50, 75, 80, 85, 90, 95, 96, 97, 98, 99, or 100%. In some embodiments, the low-molecular weight impurities each have a molecular weight of less than about 500, 400, 300, 200, 100, 50, 40, 30, 20, or 10 Daltons. In some embodiments, the low-molecular weight impurities comprise salts and/or trace solvents.

In some embodiments, the method reduces the level of the double-stranded nucleic acid by no more than 0.1, 0.5, 1, 2, 3, 4, 5, 6, 7, 8, 9, 10, 15, 20, 30, 40, or 50%. In some embodiments, the ratio of the concentration of the double-stranded nucleic acid in the retentate and the concentration of the double-stranded nucleic acid in the mixture is at least 0.75, 0.8, 0.85, 0.9, 0.95, 0.96, 0.97, 0.98, 0.99, 0.995, or 0.999. In some embodiments, the ratio of the concentration of the double-stranded nucleic acid in the retentate and the concentration of the double-stranded nucleic acid in the mixture is a ratio of the double-stranded nucleic acid in the retentate and the concentration of the double-stranded nucleic acid in the mixture prior to a purification step (e.g., prior to an ultra-filtration step).

In some embodiments, the method reduces the level of the single-stranded nucleic acid by at least 1, 2, 3, 4, 5, 6, 7, 8, 9, 10, 15, 20, 30, 40, 50, 60, 70, 80, or 90%. In some embodiments, the ratio of the concentration of the single-stranded nucleic acid in the retentate and the concentration of the single-stranded nucleic acid in the mixture is at most 0.9, 0.85, 0.8, 0.75, 0.7, 0.65, 0.6, 0.55, 0.5, 0.45, 0.4, 0.35, 0.3, 0.25, 0.2, 0.15, 0.1, 0.05, or 0.01. In some embodiments, the ratio of the concentration of the single-stranded nucleic acid in the retentate and the concentration of the single-stranded nucleic acid in the mixture is a ratio of the single-stranded nucleic acid in the retentate and the concentration of the single-stranded nucleic acid in the mixture prior to a purification step (e.g., prior to an ultra-filtration step).

In some embodiments, the method manufactures purified double-stranded nucleic acid that achieves a standard for purity. In some embodiments, the standard for purity includes the retentate including at least 50, 55, 60, 65, 70, 75, 80, 85, 90, 91, 92, 93, 94, 95, 96, 97, 98, or 99% double-stranded nucleic acid (e.g., and comprising less than 50, 45, 40, 35, 30, 25, 20, 15, 10, 9, 8, 7, 6, 5, 4, 3, 2, or 1% single-stranded nucleic acid). In some embodiments, the standard for purity includes the retentate comprising 50-100, 55-100, 60-100, 65-100, 70-100, 75-100, 80-100, 85-100, 90-100, 91-100, 92-100, 93-100, 94-100, 95-100, 96-100, 97-100, 98-100, 99-100, 80-99, 85-99, 90-99, 91-99, 92-99, 93-99, 94-99, 95-99, 96-99, 97-99, or 98-99% double-stranded nucleic acid. In some embodiments, the standard for purity comprises the retentate comprising 1-50, 1-45, 1-40, 1-35, 1-30, 1-25, 1-20, 1-15, 1-10, 1-9, 1-8, 1-7, 1-6, 1-5, 1-4, 1-3, or 1-2% single-stranded nucleic acid. In some embodiments, the method achieves the standard for purity in no more than 25, 24, 23, 22, 21, or 20 DTVs.

### A. Purified Double-Stranded Nucleic Acids

This disclosure is directed, in part, to methods of producing (e.g., manufacturing) a double-stranded nucleic acid. Generally, a method of the present disclosure includes, for example, one or more steps that selectively retain the double-stranded nucleic acid from a mixture comprising other mixture components.

In some embodiments, a method of producing (e.g., manufacturing) a purified double-stranded nucleic acid comprises one or more steps that selectively retain the double-stranded nucleic acid from a mixture (e.g., a mixture comprising other mixture components (e.g., not the desired product (e.g., a single-stranded nucleic acid), a salt (e.g., a halide or mineral salt), a buffer, a production reagent or a byproduct (e.g., a truncated nucleic acid sequence or a non-desired nucleic acid sequence), a shorter length double-stranded nucleic acid (e.g., shorter than a first and/or second single-stranded nucleic acid of interest), an organic solvent (e.g., acetonitrile, ethanol, dimethylformamide, DMSO, toluene, pyridine, or lutidine), a thiolation reagent (e.g., xanthane, hydride, or PADS), a capping reagent (e.g., acetic anhydride or NMI), a coupling reagent (e.g., phosporamidite or ETT), a detritylation reagent (e.g., dichloroacetic acid), a deprotection reagent (e.g., diethylamine, methylamine, or ammonia))), and/or a conjugation reagent (e.g carboxylic acid, carboxylic ester, carbonate, and carboxylic acid activator reagents).

In some embodiments, the mixture comprises a double-stranded nucleic acid comprising a first single-stranded nucleic acid and a second single-stranded nucleic acid and a single-stranded nucleic acid, e.g., the first single-stranded nucleic acid in single-stranded form, the second single-stranded nucleic acid in single-stranded form, or the first single-stranded nucleic acid in single-stranded form and the second single-stranded nucleic acid in single-stranded form. In some embodiments, the method of producing (e.g., manufacturing) a purified double-stranded nucleic acid comprises subjecting the mixture (e.g., a mixture described herein) to an ultra-filtration step that selectively retains double-stranded nucleic acid comprising a first and second single-stranded nucleic acids and not the single-stranded nucleic acids (e.g., the first and second single-stranded nucleic acids in single-stranded form). In some embodiments, the method of producing (e.g., manufacturing) purified double-stranded nucleic acid comprises harvesting the retentate after subjecting the mixture to an ultra-filtration step. In some embodiments, the retentate comprises double-stranded nucleic acid comprising a first and second single-stranded nucleic acids and not the single-stranded nucleic acids (e.g., the first and second single-stranded nucleic acids in single-stranded form). In some embodiments, a method of producing (e.g., manufacturing) a purified double-stranded nucleic acid comprising a first and second single-stranded nucleic acids comprises separating the double-stranded nucleic acid from the first single-stranded nucleic acid in single-stranded form and/or the second single-stranded nucleic acid in single-stranded form.

In some embodiments, the double-stranded nucleic acid is at least 10 base pairs long, e.g., at least 11, 12, 13, 14, 15, 16, 17, 18, 19, 20, 21, 22, 23, 24, 25, 26, 27, 28, 29, 30, 40, 50, 60, 70, 80, 90, 100, 110, 120, 130, 140, 150, 160, 170, 180, 190, or 200 base pairs long. In some embodiments, the double-stranded nucleic acid is no more than 100 base pairs long, e.g., no more than 80, 60, 50, 45, 40, 35, or 30 base pairs long. In some embodiments, the double-stranded nucleic acid is at least about 100, 110, 120, 130, 140, 150, 160, 170, 180, 190 to about 200 base pairs. In some embodiments, the double-stranded nucleic acid is a portion of a single stranded nucleic acid and is at least about 100, 110, 120, 130, 140, 150, 160, 170, 180, 190 to about 200 base pairs. In some embodiments, the double-stranded nucleic acid is at least 10, 11, 12, 13, 14, 15, 16, 17, 18, 19, 20, 21, 22, 23, 24, 25, 26, 27, 28, 29, or 30 base pairs long and optionally no more than 100, 80, 60, 50, 45, 40, 35, or 30 base pairs long.

In some embodiments, the double-stranded nucleic acid is 10-50 base pairs long, e.g., 12-50, 14-50, 16-50, 18-50, 20-50, 22-50, 24-50, 26-50, 28-50, 30-50, 32-50, 34-50, 36-50, 38-50, 40-50, 42-50, 44-50, 46-50, 48-50, 10-45, 12-45, 14-45, 16-45, 18-45, 20-45, 22-45, 24-45, 26-45, 28-45, 30-45, 32-45, 34-45, 36-45, 38-45, 40-45, 42-45, 10-40, 12-40, 14-40, 16-40, 18-40, 20-40, 22-40, 24-40, 26-40, 28-40, 30-40, 32-40, 34-40, 36-40, 38-40, 10-35, 12-35, 14-35, 16-35, 18-35, 20-35, 22-35, 24-35, 26-35, 28-35, 30-35, 32-35, 10-30, 12-30, 14-30, 16-30, 18-30, 20-30, 22-30, 24-30, 26-30, 28-30, 10-25, 12-25, 14-25, 16-25, 18-25, 20-25, 22-25, 10-20, 12-20, 14-20, 16-20, 18-20, 10-15, or 12-15 base pairs long.

In some embodiments, the double-stranded nucleic acid is 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides long.

In some embodiments, the double-stranded nucleic acid is 100-200 base pairs long, e.g., 100-110, 110-120, 120-130, 130-140, 140-150, 150-160, 160-170, 170-180, 180-190, or 190-200 base pairs long.

In some embodiments, the double-stranded nucleic acid comprises a sequence having at least 75%, 80%, 85%, 90%, 95%, 96%, 97%, 98%, 99%, or 100% sequence identity to a nucleic acid sequence of interest (e.g., the sequence of a target gene, non-coding RNA, primer, or component for molecule assembly). In some embodiments, the double-stranded nucleic acid comprises a sequence differing by no more than 1, 2, 3, 4, 5, 6, 7, 8, 9, or 10 nucleotides from a nucleic acid sequence of interest (e.g., the sequence of a target gene, non-coding RNA, primer, or component for molecule assembly).

In some embodiments, the double-stranded nucleic acid comprises a sequence having at least 75%, 80%, 85%, 90%, 95%, 96%, 97%, 98%, 99%, or 100% sequence complementarity to a nucleic acid sequence of interest (e.g., the sequence of a target gene, non-coding RNA, primer, or component for molecule assembly). In some embodiments, the double-stranded nucleic acid comprises a sequence differing by no more than 1, 2, 3, 4, 5, 6, 7, 8, 9, or 10 nucleotides from a sequence complimentary to a nucleic acid sequence of interest (e.g., the sequence of a target gene, non-coding RNA, primer, or component for molecule assembly).

In some embodiments, the length of at least one strand of the double-stranded nucleic acid and the length of the single-stranded nucleic acid are the same. In some embodiments, the single-stranded nucleic acid is a component of the double-stranded nucleic acid (e.g., the double-stranded nucleic acid comprises a copy of the single-stranded nucleic acid).

In some embodiments, the double-stranded nucleic acid includes a blunt end (e.g., one blunt end). In some embodiments, the double-stranded nucleic acid includes two blunt ends. In some embodiments, the double-stranded nucleic acid includes a single-stranded portion (e.g., a terminal overhang, e.g., on one end). In some embodiments, the double-stranded nucleic acid includes a single-stranded portion (e.g., a terminal overhang, e.g., on both ends).

In some embodiments, the double-stranded nucleic acid comprises a conjugate group. In some embodiments, the double-stranded nucleic acid comprises a conjugate group, e.g., that is or comprises PEG (polyethylene glycol), an amino linker, GalNAc, or a glycol spacer.

In some embodiments, the double-stranded nucleic acid includes a mismatch (e.g., at one of the ends of the double-stranded nucleic acid, within 1-2 nucleotides of one of the ends of the double stranded nucleic acid, or between the ends of the double-stranded nucleic acids).

In some embodiments, the melting temperature of the double-stranded nucleic acid in the mixture is between about 20-85° C., e.g., between about 25-80, 30-75, 35-70, 40-65, 45-60, 20-25, 25-30, 30-35, 35-40, 40-45, 45-50, 50-55, 55-60, 60-65, 65-70, 70-75, 75-80, or 80-85° C. In some embodiments, the melting temperature of the double-stranded nucleic acid in the mixture is related to concentration of salt in the mixture. In some embodiments, the relationship between the melting temperature of the double-stranded nucleic acid in the mixture and the concentration of a salt (e.g., a cation, e.g., sodium ion) in the mixture is determined according to a method as described in Owczarzy et al. (2004, Biochemistry 43:3537-3554; incorporated herein by reference in its entirety).

In some embodiments, the ratio of the concentration of the double-stranded nucleic acid in the retentate and the concentration of the double-stranded nucleic acid in the mixture is at least 0.50, 0.55, 0.60, 0.65, 0.70, 0.75, 0.80, 0.85, 0.90, 0.95, 0.96, 0.97, 0.98, 0.99, 0.995, 0.999, 1.00, 1.01, 1.05, 1.10, 1.15, 1.20, 1.25, 1.30, 1.35, 1.40, 1.45, 1.50, or 1.55. In some embodiments, the quantity of the double-stranded nucleic acid in the retentate is at least 500, 525, 550, 575, 600, 625, 650, 675, 700, 725, 750, 775, 800, 825, 850, 875, 900, 925, 950, 975, 1000, 1025, 1050, 1075, 1100, 1125, 1150, 1175, or 1200 OD/mL. In some embodiments, the quantity of the double-stranded nucleic acid in the retentate is between about 500-1200 OD/mL (e.g., between about 500-550, 550-600, 600-650, 650-700, 700-750, 750-800, 800-850, 850-900, 900-950, 950-1000, 1000-1050, 1050-1100, 1100-1150, or 1150-1200 OD/mL). In some embodiments, the quantity of the double-stranded nucleic acid in the retentate is at least 20, 22, 24, 26, 28, 30, 32, 34, 36, 38, 40, 42, 44, 46, or 48 mg/mL. In some embodiments, the quantity of the double-stranded nucleic acid in the retentate is between about 20-48 mg/mL (e.g., between about 20-25, 25-30, 30-35, 35-40, 40-45, or 45-48 mg/mL).

In some embodiments, the ratio of the quantity of the double-stranded nucleic acid in the retentate and the quantity of the double-stranded nucleic acid in the mixture is at least 0.50, 0.55, 0.60, 0.65, 0.70, 0.75, 0.80, 0.85, 0.90, 0.95, 0.96, 0.97, 0.98, 0.99, 0.995, 0.999, or 1.00. In some embodiments, the ratio of the quantity of the single-stranded nucleic acid in the retentate and the quantity of the single-stranded nucleic acid in the mixture is at most 0.50, 0.45, 0.40, 0.35, 0.30, 0.25, 0.20, 0.15, 0.10, 0.05, 0.05, 0.04, 0.03, 0.02, or 0.01.

In some embodiments, the ratio of double-stranded nucleic acid relative to single-stranded nucleic acid in the retentate (e.g., as determined by non-denaturing HPLC) increases over time and/or over diafiltration total volume (DTV). In certain embodiments, the ratio of double-stranded nucleic acid relative to single-stranded nucleic acid in the retentate (e.g., as determined by non-denaturing HPLC) is at least 50:50, 70:25, 70:30, 75:20, 75:25 80:15, 80:20, 85:10, 85:15, 90:5, 90:10, 90.3:5, 90.3, 9.7, 90.4:4.8, 90.4:9.6, 90.9:4.8, 90.9:9.1, 91.1:4.6, 91.1:8.9, 91.2:4.5, 91.2:8.2, 91.1:4.4, 91.1:8.9, 91.2:4.4, 91.2:8.8, 91.3:4.1, 91.3:8.7, 91.5:3.9, 91.5:8.5, 92:3, 92:8, 94:5, 94:6, 95:2, 95:5, 98:1, 98:2, 99:1, or 99.5:0.5.

In some embodiments, the ratio of the concentration of the double-stranded nucleic acid in the retentate and the concentration of the single-stranded nucleic acid in the mixture is at least 0.50, 0.55, 0.60, 0.65, 0.70, 0.75, 0.80, 0.85, 0.90, 0.95, 0.96, 0.97, 0.98, 0.99, 0.995, 0.999, 1.00, 1.01, 1.05, 1.10, 1.15, 1.20, 1.25, 1.30, 1.35, 1.40, 1.45, 1.50, or 1.55. In some embodiments, the ratio of the quantity of the single-stranded nucleic acid in the retentate and the quantity of the double-stranded nucleic acid in the mixture is at least 0.50, 0.55, 0.60, 0.65, 0.70, 0.75, 0.80, 0.85, 0.90, 0.95, 0.96, 0.97, 0.98, 0.99, 0.995, 0.999, 1.00, 1.01, 1.05, 1.10, 1.15, 1.20, 1.25, 1.30, 1.35, 1.40, 1.45, 1.50, or 1.55.

In some embodiments, providing the double-double stranded nucleic acid includes annealing a first single-stranded nucleic acid and a second single-stranded nucleic acid. Individual purified aqueous oligonucleotide matrices are introduced into a vessel equipped with mixing, heating/cooling and nitrogen atmosphere capability. The amounts of each single-stranded nucleic acid are added in equivalent portions to a desired molar ratio (e.g., equivalent or excess) is achieved. The ratio is verified by non-denaturing analytical chromatography (e.g. HPLC, SEC). The mixture of aqueous single-stranded nucleic acids is then subject to thermal cycling (e.g., annealing) to facilitate hybridization to form the duplex nucleic acid. Annealing is performed by first cooling the solutions below room temperature (˜5° C.-10° C. (e.g., about 5, about 6, about 7, about 8, about 9, or about 10° C.)) then heating to an elevated temperature at a controlled heating rate (gradient) to achieve a temperature that is close to the Tm of the hybridized duplex and held for a short amount of time at this elevated temperature prior to cooling at a controlled rate (gradient) back to below room temperature (˜5° C.-10° C. (e.g., (e.g., about 5° C., about 6° C., about 7° C., about 8° C., about 9° C., or about 10° C.)) to arrive at concentrated aqueous solution of duplex nucleic acid.

### B. Purified Single-Stranded Nucleic Acids

This disclosure is directed, in part, to methods of purifying a single-stranded nucleic acid. Generally, a method of the present disclosure includes, for example, one or more steps that selectively retain double-stranded nucleic acid from a mixture comprising the single-stranded nucleic acid, the double-stranded nucleic acids, and/or other mixture components. In some embodiments, the method of purifying a single-stranded nucleic acid does not include the synthesis of the single-stranded nucleic acid but nevertheless includes one or more steps to remove the single-stranded nucleic acids from a mixture.

In some embodiments, a method of manufacturing a purified single-stranded nucleic acid comprises one or more steps that selectively retain the double-stranded nucleic acid from a mixture (e.g., a mixture comprising other mixture components (e.g., not the desired product (e.g., a single-stranded nucleic acid), a salt (e.g., a halide or mineral salt), a buffer, a production reagent or a byproduct (e.g., a truncated nucleic acid sequence or a non-desired nucleic acid sequence), a shorter length single-stranded nucleic acid (e.g., shorter than a first and/or second single-stranded nucleic acid of interest), an organic solvent (e.g., acetonitrile, ethanol, dimethylformamide, DMSO, toluene, pyridine, or lutidine), a thiolation reagent (e.g., xanthane, hydride, or PADS), a capping reagent (e.g., acetic anhydride or NMI), a coupling reagent (e.g., phosporamidite or ETT), a detritylation reagent (e.g., dichloroacetic acid), a deprotection reagent (e.g., diethylamine, ethylene diamine, methylamine, or ammonia))), and/or a conjugation reagent (e.g carboxylic acid, carboxylic ester, carbonate, amine, and carboxylic acid activator reagents). In some embodiments, the single-stranded nucleic acid is within a permeate. In some embodiments, the single-stranded nucleic acid is within a permeate and the permeate is collected.

In some embodiments, the first single-stranded nucleic acid is at least 10 nucleotides long, e.g., at least 11, 12, 13, 14, 15, 16, 17, 18, 19, 20, 21, 22, 23, 24, 25, 26, 27, 28, 29, 30, 40, 50, 60, 70, 80, 90, 100, 110, 120, 130, 140, 150, 160, 170, 180, 190, or 200 nucleotides long. In some embodiments, double-stranded nucleic acid is at least about 100, 110, 120, 130, 140, 150, 160, 170, 180, 190 to about 200 nucleotides. In some embodiments, double-stranded nucleic acid is a portion of a single stranded nucleic acid and is at least about 100, 110, 120, 130, 140, 150, 160, 170, 180, 190 to about 200 nucleotides. In some embodiments, the first single-stranded nucleic acid is at least 10, 11, 12, 13, 14, 15, 16, 17, 18, 19, 20, 21, 22, 23, 24, 25, 26, 27, 28, 29, 30, 40, 50, 60, 70, 80, 90, 100, 110, 120, 130, 140, 150, 160, 170, 180, 190, or 200 nucleotides long and optionally is no more than 200, 190, 180, 170, 160, 150, 140, 130, 120, 110, 100, 80, 60, 50, 45, 40, 35, or 30 nucleotides long. In some embodiments, the first single-stranded nucleic acid is no more than 100 nucleotides long, e.g., no more than 80, 60, 50, 45, 40, 35, or 30 nucleotides long.

In some embodiments, the first single-stranded nucleic acid is 10-50 nucleotides long, e.g., 12-50, 14-50, 16-50, 18-50, 20-50, 22-50, 24-50, 26-50, 28-50, 30-50, 32-50, 34-50, 36-50, 38-50, 40-50, 42-50, 44-50, 46-50, 48-50, 10-45, 12-45, 14-45, 16-45, 18-45, 20-45, 22-45, 24-45, 26-45, 28-45, 30-45, 32-45, 34-45, 36-45, 38-45, 40-45, 42-45, 10-40, 12-40, 14-40, 16-40, 18-40, 20-40, 22-40, 24-40, 26-40, 28-40, 30-40, 32-40, 34-40, 36-40, 38-40, 10-35, 12-35, 14-35, 16-35, 18-35, 20-35, 22-35, 24-35, 26-35, 28-35, 30-35, 32-35, 10-30, 12-30, 14-30, 16-30, 18-30, 20-30, 22-30, 24-30, 26-30, 28-30, 10-25, 12-25, 14-25, 16-25, 18-25, 20-25, 22-25, 10-20, 12-20, 14-20, 16-20, 18-20, 10-15, or 12-15 nucleotides long.

In some embodiments, the first single-stranded nucleic acid is 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides long.

In some embodiments, the second single-stranded nucleic acid is at least 10 nucleotides long, e.g., 11, 12, 13, 14, 15, 16, 17, 18, 19, 20, 21, 22, 23, 24, 25, 26, 27, 28, 29, 30, 40, 50, 60, 70, 80, 90, 100, 110, 120, 130, 140, 150, 160, 170, 180, 190, or 200 nucleotides long. In some embodiments, the second single-stranded nucleic acid is no more than 100 nucleotides long, e.g., no more than 80, 60, 50, 45, 40, 35, or 30 nucleotides long. In some embodiments, the second single-stranded nucleic acid is at least 10, 11, 12, 13, 14, 15, 16, 17, 18, 19, 20, 21, 22, 23, 24, 25, 26, 27, 28, 29, 30, 40, 50, 60, 70, 80, 90, 100, 110, 120, 130, 140, 150, 160, 170, 180, 190, or 200 nucleotides long and optionally is no more than 200, 190, 180, 170, 160, 150, 140, 130, 120, 110, 100, 80, 60, 50, 45, 40, 35, or 30 nucleotides long.

In some embodiments, the second single-stranded nucleic acid is 10-50 nucleotides long, e.g., 12-50, 14-50, 16-50, 18-50, 20-50, 22-50, 24-50, 26-50, 28-50, 30-50, 32-50, 34-50, 36-50, 38-50, 40-50, 42-50, 44-50, 46-50, 48-50, 10-45, 12-45, 14-45, 16-45, 18-45, 20-45, 22-45, 24-45, 26-45, 28-45, 30-45, 32-45, 34-45, 36-45, 38-45, 40-45, 42-45, 10-40, 12-40, 14-40, 16-40, 18-40, 20-40, 22-40, 24-40, 26-40, 28-40, 30-40, 32-40, 34-40, 36-40, 38-40, 10-35, 12-35, 14-35, 16-35, 18-35, 20-35, 22-35, 24-35, 26-35, 28-35, 30-35, 32-35, 10-30, 12-30, 14-30, 16-30, 18-30, 20-30, 22-30, 24-30, 26-30, 28-30, 10-25, 12-25, 14-25, 16-25, 18-25, 20-25, 22-25, 10-20, 12-20, 14-20, 16-20, 18-20, 10-15, or 12-15nucleotides long.

In some embodiments, the second single-stranded nucleic acid is 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides long.

In some embodiments, the length of at least one strand of the double-stranded nucleic acid and the length of the single-stranded nucleic acid are the same. In some embodiments, the single-stranded nucleic acid is a component of the double-stranded nucleic acid (e.g., the double-stranded nucleic acid comprises a copy of the single-stranded nucleic acid).

### III. Apparatus

In some embodiments, the ultra-filtration step comprises applying the mixture (e.g., a mixture described herein) to a filtration apparatus (e.g., an apparatus comprising a barrier (e.g., a membrane, e.g., a semi-permeable membrane)). In some embodiments, the apparatus comprises a first chamber, a second chamber, and/or a filtration element, e.g., a barrier (e.g., a membrane (e.g., a semi-permeable membrane)), e.g., as described herein, disposed between the first and second chambers. In some embodiments, the first chamber is configured to hold a mixture (e.g., a mixture described herein) to be filtered, and optionally to capture retentate. In some embodiments, the second chamber is configured to capture a permeate. In some embodiments, the mixture comprises double-stranded nucleic acid (e.g., comprising a first single-stranded nucleic acid and a second single-stranded nucleic acid) and single-stranded nucleic acid (e.g., the first single-stranded nucleic acid in single-stranded form, the second single-stranded nucleic acid in single-stranded form, or the first single-stranded nucleic acid in single-stranded form and the second single-stranded nucleic acid in single-stranded form). In some embodiments, the filtration element (e.g., a barrier (e.g., a membrane, e.g., a semi-permeable membrane)) selectively retains double-stranded nucleic acid (e.g., comprising the first and second single-stranded nucleic acids) and not single-stranded nucleic acid (e.g., the first and/or second single stranded nucleic acids in single-stranded form). In some embodiments, the apparatus does not substantially retain one or more other mixture components (e.g., the apparatus does not retain a detectable amount of one or more other mixture components), e.g., as described herein.

In some embodiments, the apparatus is pressurized (e.g., at a pressure of about 5-40 psi, in some instances at medium psi range (˜15-30 psi)) to concentrate the matrix on the retentate side of the membrane (e.g., the semi-permeable membrane). Process water may then, in some embodiments, be introduced under pressure to facilitate filtration (e.g., diafiltration) of other mixture components.

In some embodiments, the ultra-filtration step produces a retentate and a permeate. In some embodiments, the retentate is enriched for a desired molecule of interest (e.g., a double-stranded nucleic acid or a single-stranded nucleic acid), e.g., by at least 2, 3, 4, 5, 6, 7, 8, 9, 10, 20, 30, 40, 50, 60, 70, 80, 90, or 100%, relative to the mixture subjected to ultra-filtration. In some embodiments, the retentate is enriched for a double-stranded nucleic acid, e.g., by at least 2, 3, 4, 5, 6, 7, 8, 9, 10, 20, 30, 40, 50, 60, 70, 80, 90, or 100%, relative to the mixture subjected to ultra-filtration. In some embodiments, the permeate is enriched for a single-stranded nucleic acid, e.g., by at least 2, 3, 4, 5, 6, 7, 8, 9, 10, 20, 30, 40, 50, 60, 70, 80, 90, or 100%, relative to the mixture subjected to ultra-filtration.

In some embodiments, the desired molecule of interest (e.g., a double-stranded nucleic acid or a single-stranded nucleic acid) is enriched from 2% to at least 90% purity, from 5% to at least 90% purity, from 10% to at least 90% purity, from 15% to at least 90% purity, from 20% to at least 90% purity, from 25% to at least 90% purity, from 30% to at least 90% purity, from 35% to at least 90% purity, from 40% to at least 90% purity, from 45% to at least 90% purity, from 50% to at least 90% purity, from 55% to at least 90% purity, from 60% to at least 90% purity, from 65% to at least 90% purity, from 70% to at least 90% purity, from 75% to at least 90% purity, from 80% to at least 90% purity, from 85% to at least 90% purity, from 90% to at least 92.5% purity, from 95% to at least 97.5% purity, or from 97.5% to at least 99% purity. In some embodiments,, the desired molecule of interest is enriched from 50% to at least 90% purity, from 55% to at least 90% purity, from 60% to at least 90% purity, from 65% to at least 90% purity, from 70% to at least 90% purity, from 75% to at least 90% purity, from 80% to at least 90% purity, from 85% to at least 90% purity, from 90% to at least 92.5% purity, from 92.5% to at least 95% purity, from 95% to at least 97.5% purity, or from 97.5% to at least 99% purity.

In some embodiments, the purity of the desired molecule of interest (e.g., a double-stranded nucleic acid or a single-stranded nucleic acid) is increased by about 1, 2, 3, 4, 5, 6, 7, 8, 9, 10, 20, 30, 40, 50, 60, 70, 80, 90, 100, 200, 300, 400, 500, 600, 700, 800, 900, 1,000, 2,000, 3,000, 4,000, or 5,000 fold after the ultra-filtration.

In some embodiments, the ultra-filtration step produces a retentate and a permeate. In some embodiments, the retentate is enriched for a desired molecule of interest (e.g., a double-stranded nucleic acid or a single-stranded nucleic acid), e.g., by at least 2, 3, 4, 5, 6, 7, 8, 9, 10, 20, 30, 40, 50, 60, 70, 80, 90, or 100%, relative to the permeate. In some embodiments, the retentate is enriched for a double-stranded nucleic acid, e.g., by at least 2, 3, 4, 5, 6, 7, 8, 9, 10, 20, 30, 40, 50, 60, 70, 80, 90, or 100%, relative to the permeate. In some embodiments, the permeate is enriched for a single-stranded nucleic acid, e.g., by at least 2, 3, 4, 5, 6, 7, 8, 9, 10, 20, 30, 40, 50, 60, 70, 80, 90, or 100%, relative to the permeate.

In some embodiments, the retentate comprises a different concentration of one or more of a salt, a buffer, or a production reagent or byproduct than the permeate, the mixture, or the permeate and the mixture. In some embodiments, the retentate has a lower concentration of one or more of a salt, a buffer, or a production reagent or byproduct than the permeate, the mixture, or the permeate and the mixture.

In some embodiments, the second chamber includes a permeate. In some embodiments, the second chamber includes a permanent, e.g., includes single-stranded nucleic acid. In some embodiments, the second chamber retains the permeate. In some embodiments, the second chamber does not retain permeate. In some embodiments, the second chamber does not retain permeate and the permeate flows from the second chamber and is discarded.

In some embodiments, the single-stranded nucleic acid in the permeate is composed primarily of sense strands (e.g., wherein at least 75%, 80%, 85%, 90%, 95%, 96%, 97%, 98%, 99%, 99.5%, 99.9%, or 100% of the single-stranded nucleic acid in the permeate consists of sense strands). In some embodiments, the single-stranded nucleic acid in the permeate is composed primarily of antisense strands (e.g., wherein at least 75%, 80%, 85%, 90%, 95%, 96%, 97%, 98%, 99%, 99.5%, 99.9%, or 100% of the single-stranded nucleic acid in the permeate consists of antisense strands).

### A. Membranes

In some embodiments, the filtration apparatus or filtration element membrane comprises one or more of: polysulfone, polypropylene, cellulose acetate, polyactic acid, nitrocellulose, mix cellulose ester cellulose nitrate, regenerated cellulose, polyethersulfone, polyamide, cellulose derivative (HYDROSART®) polyvinylidene fluoride, polytetra-fluoroethylene, or polycarbonate track etched membranes. In some embodiments, the filtration apparatus or filtration element is or comprises a hollow fiber, tubular, spiral-wound, cassette, plate, or frame membrane type filter.

### 1. Molecular Weight Cutoffs (MWCO)

In some embodiments, the barrier (e.g., a membrane, e.g., a semi-permeable membrane) comprises a molecular weight cutoff (MWCO). In some embodiments, the barrier having a MWCO selectively retains double-stranded nucleic acids (e.g., preferentially over single-stranded nucleic acids). In some embodiments, the barrier having a MWCO selectively permits single-stranded nucleic acids to flow through (e.g., preferentially over double-stranded nucleic acids). In some embodiments, the MWCO is at least about 1, 2, 3, 4, 5, 6, 7, 8, 9, or 10 kilodaltons less than the MW of the double-stranded nucleic acid. In some embodiments, the MWCO is no more than about 300, 250, 200, 150, 100, 50, 40, 30, 20, 15, 10, 9, 8, 7, 6, 5, 4, 3, or 2 kilodaltons less than the MW of the double-stranded nucleic acid. In some embodiments, the MWCO is at least about 1, 2, 3, 4, 5, 6, 7, 8, 9, or 10 kilodaltons less than the MW of the double-stranded nucleic acid and is no more than about 20, 15, 10, 9, 8, 7, 6, 5, 4, 3, or 2 kilodaltons less than the MW of the double-stranded nucleic acid. In some embodiments, the MWCO is at least about 1, 2, 3, 4, 5, 6, 7, 8, 9, 10, 15, 20, 25, 30, 50, 100, 150, 200, 250, 300 kilodaltons greater than the molecular weight of the single-stranded nucleic acid (e.g., the first single-stranded nucleic acid, the second single-stranded nucleic acid, or both). In some embodiments, the MWCO is at least about 1, 2, 3, 4, 5, 6, 7, 8, 9, 10, 15, 20, 25, 30, 50, 100, 150, 200, 250, 300 kilodaltons less than the molecular weight of the double-stranded nucleic acid. In some embodiments, the MWCO is at least about 1, 2, 3, 4, 5, 6, 7, 8, 9, 10, 15, 20, 25, 30, 50, 100, 150, 200, 250, 300 kilodaltons greater than the molecular weight of the single-stranded nucleic acid (e.g., the first single-stranded nucleic acid, the second single-stranded nucleic acid, or both) and is at least about 1, 2, 3, 4, 5, 6, 7, 8, 9, 10, 15, 20, 25, 30, 50, 100, 150, 200, 250, 300 kilodaltons less than the molecular weight of the double-stranded nucleic acid. In some embodiments, the MWCO is about 1-30, 2-30, 3-30, 4-30, 5-30, 6-30, 7-30, 8-30, 9-30, 10-30, 15-30, 20-30, 25-30, 1-25, 2-25, 3-25, 4-25, 5-25, 6-25, 7-25, 8-25, 9-25, 10-25, 15-25, 20-25, 1-20, 2-20, 3-20, 4-20, 5-20, 6-20, 7-20, 8-20, 9-20, 10-20, 15-20, 1-15, 2-15, 3-15, 4-15, 5-15, 6-15, 7-15, 8-15, 9-15, 10-15, 1-10, 2-10, 3-10, 4-10, 5-10, 6-10, 7-10, 8-10, 9-10, 1-9, 2-9, 3-9, 4-9, 5-9, 6-9, 7-9, 8-9, 1-8, 2-8, 3-8, 4-8, 5-8, 6-8, 7-8, 1-7, 2-7, 3-7, 4-7, 5-7, 6-7, 1-6, 2-6, 3-6, 4-6, 5-6, 1-5, 2-5, 3-5, 4-5, 1-4, 2-4, 3-4, 1-3, 2-3, or 1-2 kilodaltons greater than the molecular weight of the single-stranded nucleic acid (e.g., the first single-stranded nucleic acid, the second single-stranded nucleic acid, or both). In some embodiments, the MWCO is about 1-30, 2-30, 3-30, 4-30, 5-30, 6-30, 7-30, 8-30, 9-30, 10-30, 15-30, 20-30, 25-30, 1-25, 2-25, 3-25, 4-25, 5-25, 6-25, 7-25, 8-25, 9-25, 10-25, 15-25, 20-25, 1-20, 2-20, 3-20, 4-20, 5-20, 6-20, 7-20, 8-20, 9-20, 10-20, 15-20, 1-15, 2-15, 3-15, 4-15, 5-15, 6-15, 7-15, 8-15, 9-15, 10-15, 1-10, 2-10, 3-10, 4-10, 5-10, 6-10, 7-10, 8-10, 9-10, 1-9, 2-9, 3-9, 4-9, 5-9, 6-9, 7-9, 8-9, 1-8, 2-8, 3-8, 4-8, 5-8, 6-8, 7-8, 1-7, 2-7, 3-7, 4-7, 5-7, 6-7, 1-6, 2-6, 3-6, 4-6, 5-6, 1-5, 2-5, 3-5, 4-5, 1-4, 2-4, 3-4, 1-3, 2-3, or 1-2 kilodaltons less than the molecular weight of the double-stranded nucleic acid. In particular embodiments, the MWCO is at least about 2 kilodaltons greater than the molecular weight of the single-stranded nucleic acid (e.g., the first single-stranded nucleic acid, the second single-stranded nucleic acid, or both). In particular embodiments, the MWCO is at least about 5 kilodaltons less than the molecular weight of the double-stranded nucleic acid. In certain embodiments, the MWCO is at least about 2 kilodaltons greater than the molecular weight of the single-stranded nucleic acid (e.g., the first single-stranded nucleic acid, the second single-stranded nucleic acid, or both) and is at least about 5 kilodaltons less than the molecular weight of the double-stranded nucleic acid.

In some embodiments, the MWCO is about 0.5-100, 0.5-80, 0.5-60, 0.5-50, 0.5-40, 0.5-30, 0.5-20, 0.5-15, 0.5-10, 0.5-8, 0.5-6, 0.5-4, 0.5-3, 0.5-2, 0.5-1, 1-100, 1-80, 1-60, 1-50, 1-40, 1-30, 1-20, 1-15, 1-10, 1-8, 1-6, 1-4, 1-3, 1-2, 2-100, 2-80, 2-60, 2-50, 2-40, 2-30, 2-20, 2-15, 2-10, 2-8, 2-6, 2-4, 2-3, 3-100, 3-80, 3-60, 3-50, 3-40, 3-30, 3-20, 3-15, 3-10, 3-8, 3-6, 3-4, 4-100, 4-80, 4-60, 4-50, 4-40, 4-30, 4-20, 4-15, 4-10, 4-8, 4-6, 6-100, 6-80, 6-60, 6-50, 6-40, 6-30, 6-20, 6-15, 6-10, 6-8, 8-100, 8-80, 8-60, 8-50, 8-40, 8-30, 8-20, 8-15, 8-10, 10-100, 10-80, 10-60, 10-50, 10-40, 10-30, 10-20, 10-15, 15-100, 15-80, 15-60, 15-50, 15-40, 15-30, 15-20, 20-100, 20-80, 20-60, 20-50, 20-40, 20-30, 30-100, 30-80, 30-60, 30-50, 30-40, 40-100, 40-80, 40-60, 40-50, 50-100, 50-80, 50-60, 60-80, 60-100, or 80-100 kilodaltons. In some embodiments, the MWCO is at least 100, 125, 150, 175, 200, 225, 250, 275, or 300 kilodaltons. In some embodiments, the MWCO is about 100-125, 125-150, 150-175, 175-200, 200-225, 225-250, 250-275, or 275-300 kilodaltons. In some embodiments, the MWCO is about 2-3, 3-4, 4-5, 5-6, 6-7, 7-8, 8-9, 9-10, 10-11, 11-12, 12-13, 13-14, 14-15, or 15-16 kDa. In certain embodiments, the MWCO is about 8-9, 9-10, 10-11, 11-12, 12-13, 13-14, or 14-15 kDa. In particular embodiments, the MWCO is about 8-15 kDa. In certain embodiments, the MWCO is about 10 kDa.

In some embodiments, a barrier having MWCO of about 10 kDa (e.g., about 7, 8, 9, 10, 11, 12, 13, or 14 kDa) selectively retains a double-stranded nucleic acid without substantial denaturation of the double-stranded nucleic acid (e.g., without detectable denaturation of the double-stranded nucleic acid), e.g., at a transmembrane pressure of about 15-50 psi (e.g., about 15-20, 20-25, 23-30, 25-35, 35-45, or 40-50, 55-60). In some embodiments, a barrier having MWCO of about 10 kDa (e.g., about 7, 8, 9, 10, 11, 12, 13, or 14 kDa) selectively retains a double-stranded nucleic acid, wherein less than 10%, 9%, 8%, 7%, 6%, 5%, 4%, 3%, 2%, 1%, 0.1%, 0.001%, 0.0001%, or 0.00001% of the double-stranded nucleic acid in the mixture is denatured.

### 2. Average Pore Size

In some embodiments, the barrier (e.g., a membrane, e.g., a semi-permeable membrane) comprises pores of a preselected average pore size. In some embodiments, the average pore size is an average pore size that selectively retains double-stranded nucleic acid.

In some embodiments, the average pore size is at least about 1, 1.2, 1.4, 1.6, 1.8, 2, 2.2, 2.4, 2.6, 2.8, or 3 nm and no more than 4, 3.8, 3.6, 3.4, 3.2, 3, 2.8, 2.6, 2.4, 2.2, 2, 1.8, or 1.6 nm.

In some embodiments, the average pore size is about 0.05-20, 0.1-20, 0.5-20, 1-20, 2-20, 3-20, 4-20, 5-20, 6-20, 7-20, 8-20, 9-20, 10-20, 15-20, 0.05-10, 0.1-10, 0.5-10, 1-10, 2-10, 3-10, 4-10, 5-10, 6-10, 7-10, 8-10, 9-10, 0.05-8, 0.1-8, 0.5-8, 1-8, 2-8, 3-8, 4-8, 5-8, 6-8, 7-8, 0.05-6, 0.1-6, 0.5-6, 1-6, 2-6, 3-6, 4-6, 5-6, 0.05-4, 0.1-4, 0.5-4, 1-4, 2-4, 3-4, 0.05-2, 0.1-2, 0.5-2, 1-2, 0.05-1, 0.1-1, or 0.5-1 μm.

In some embodiments, the average pore size is greater than Z (and optionally, less than 0.8Y, 0.85Y, 0.9Y, 0.95Y, 0.99Y, Y, or 1.1Y), wherein Z is the numerical average of X and Y, wherein X is the smallest pore size that will allow at least 90% (e.g., at least 91%, at least 92%, at least 93%, at least 94%, at least 95%, at least 96%, at least 97%, at least 98%, or at least 99%) of the first single-stranded nucleic acid, second single-stranded nucleic acid, or both to permeate the membrane (e.g., in less than about 2, 3, 4, 5, 6, 7, 8, 9, 10, 11, 12, 13, 14, 15, 20, 25, 27, 30, 35, 40, 45, 50, 55, 60, 63,65, 70, 75, 80, 81, 85, or 90 diafiltration volumes (DTVs)); and wherein Y is the smallest pore size that will allow at least 90% of the double-stranded nucleic acid to permeate the membrane (e.g., in less than about 2, 3, 4, 5, 6, 7, 8, 9, 10, 11, 12, 13, 14, 15, 20, 25, 27, 30, 35, 40, 45, 50, 55, 60, 63,65, 70, 75, 80, 81, 85, or 90 DTVs).

### B. Parameters

Without wishing to be bound by theory, it is contemplated that in some embodiments certain process parameters are important to determining the efficacy of a method of producing (e.g., manufacturing) an oligonucleotide (e.g., a purified double-stranded oligonucleotide and/or a purified single-stranded oligonucleotide) in combination with one another. In some embodiments, a combination of process parameters have optimal configurations in relation to one another, e.g., and not when considered in isolation. Exemplary combinations of process parameters are described below.

### C. Mixture Components

In some embodiments, the mixture includes double-stranded nucleic acid and single-stranded nucleic acid. In some embodiments, the mixture includes a first single-stranded nucleic acid, a second single-stranded nucleic acid, and a double-stranded nucleic acid including the first single-stranded nucleic acid and the second single stranded nucleic acid. In some embodiments the mixture includes a first single-stranded nucleic acid and a second single-stranded nucleic acid, where the first single-stranded nucleic acid and the second single-stranded nucleic acid are sufficiently complementary to form a double-stranded nucleic acid, and the mixture includes the double stranded nucleic acid. In some embodiments, the mixture includes a first single-stranded nucleic acid, a second single-stranded nucleic acid, and a double-stranded nucleic acid including the first single-stranded nucleic acid and the second single stranded nucleic acid and other mixture components.

In some embodiments, the mixture includes other mixture components. In some embodiments, the other mixture components include components that are not the desired product (e.g., a desired molecule of interest, e.g., present in the retentate or the permeate). In some embodiments, the product comprises double-stranded nucleic acids. In some embodiments, the product comprises single-stranded nucleic acids. In some embodiments, the other mixture components include, but are not limited to, a salt (e.g., a halide or mineral salt), a buffer, a production reagent or a byproduct (e.g., a truncated nucleic acid sequence or a non-desired nucleic acid sequence), a shorter length single-stranded nucleic acid (e.g., shorter than a first and/or second single-stranded nucleic acid of interest), an organic solvent (e.g., acetonitrile, ethanol, dimethylformamide, DMSO, toluene, pyridine, or lutidine), a thiolation reagent (e.g., xanthane, hydride, or PADS), a capping reagent (e.g., acetic anhydride or NMI), a coupling reagent (e.g., phosporamidite or ETT), a detritylation reagent (e.g., dichloroacetic acid), a deprotection reagent (e.g., diethylamine, methylamine, or ammonia), a conjugation reagent (e.g., carboxylic acid, carboxylic ester, amine, carbonate, and carboxylic acid activator reagents), or any combination thereof.

In some embodiments, the mixture includes no more than 1, 2, 3, 4, 5, 6, 7, 8, 9, 10, 20, 30, 40, 50, 60, 70, 80, 90, or 100 μl, or no more than 0.5, 1, 2, 3, 4, 5, 6, 7, 8, 9, 10, 20, 30, 40, 50, 60, 70, 80, 90, or 100 ml, or no more than 0.5, 1, 2, 3, 4, 5, 6, 7, 8, 9, 10, 20, 30, 40, 50, 60, 70, 80, 90, 100, 150, 200, 250, 300, 350, 400, 450, 500, 600, 700, 800, 900, or 1000 L (and optionally, at least 1, 2, 3, 4, 5, 6, 7, 8, 9, 10, 20, 30, 40, 50, 60, 70, 80, 90, or 100 pl, or at least 0.5, 1, 2, 3, 4, 5, 6, 7, 8, 9, 10, 20, 30, 40, 50, 60, 70, 80, 90, or 100 ml, or at least 0.5, 1, 2, 3, 4, 5, 6, 7, 8, 9, 10, 20, 30, 40, 50, 60, 70, 80, 90, 100, 150, 200, 250, 300, 350, 400, 450, 500, 600, 700, 800, 900, or 1000 L).

In some embodiments, the mixture includes at least 1, 2, 3, 4, 5, 6, 7, 8, 9, 10, 20, 30, 40, 50, 60, 70, 80, 90, or 100 μl, or at least 0.5, 1, 2, 3, 4, 5, 6, 7, 8, 9, 10, 20, 30, 40, 50, 60, 70, 80, 90, or 100 ml, or at least 0.5, 1, 2, 3, 4, 5, 6, 7, 8, 9, 10, 20, 30, 40, 50, 60, 70, 80, 90, 100, 150, 200, 250, 300, 350, 400, 450 500, 600, 700, 800, 900, or 1000 L (and optionally no more than 1, 2, 3, 4, 5, 6, 7, 8, 9, 10, 20, 30, 40, 50, 60, 70, 80, 90, or 100 μl, or no more than 0.5, 1, 2, 3, 4, 5, 6, 7, 8, 9, 10, 20, 30, 40, 50, 60, 70, 80, 90, or 100 ml, or no more than 0.5, 1, 2, 3, 4, 5, 6, 7, 8, 9, 10, 20, 30, 40, 50, 60, 70, 80, 90, 100, 150, 200, 250, 300, 350, 400, 450, 500, 600, 700, 800, 900, or 1000 L).

In some embodiments, the mixture includes: 1-100, 5-100, 10-100, 20-100, 30-100, 40-100, 50-100, 60-100, 70-100, 80-100, 90-100, 1-50, 5-50, 10-50, 20-50, 30-50, 40-50, 1-20, 5-20, 10-20, or 1-10 μl; 0.1-100, 0.5-100, 1-100, 5-100, 10-100, 20-100, 30-100, 40-100, 50-100, 60-100, 70-100, 80-100, 90-100, 0.1-50, 0.5-50, 1-50, 5-50, 10-50, 20-50, 30-50, 40-50, 0.1-20, 0.5-20, 1-20, 5-20, 10-20, 0.1-10, 0.5-10, 1-10, 0.1-1, 0.5-1, or 0.1-0.5ml; or 0.1-500, 0.5-500, 1-500, 5-500, 10-500, 20-500, 30-500, 40-500, 50-500, 60-500, 70-500, 80-500, 90-500, 100-500, 150-500, 200-500, 250-500, 300-500, 350-500, 400-500, 450-500, 0.1-250, 0.5-250, 1-250, 5-250, 10-250, 20-250, 30-250, 40-250, 50-250, 60-250, 70-250, 80-250, 90-250, 100-250, 150-250, 200-250, 0.1-100, 0.5-100, 1-100, 5-100, 10-100, 20-100, 30-100, 40-100, 50-100, 60-100, 70-100, 80-100, 90-100, 0.1-50, 0.5-50, 1-50, 5-50, 10-50, 20-50, 30-50, 40-50, 0.1-20, 0.5-20, 1-20, 5-20, 10-20, 0.1-10, 0.5-10, 1-10, 0.1-1, 0.5-1, or 0.1-0.5 L.

In some embodiments, the mixture includes a nucleic acid concentration (e.g., overall nucleic acid concentration, double-stranded nucleic acid concentration, or single-stranded nucleic acid concentration) of at least 300, 400, 500, 600, 700, 800, 900, 1000, 1100, 1200, 1300, 1400, 1500, 1600, 1700, 1800, 1900, 2000, 3000, 4000, 5000, 6000, 7000, 8000, or 9000 OD/ml (e.g., OD at 250-260 nm, e.g., 258-260 nm) (and optionally no more than 2500, 2000, 1500, 1400, 1300, 1200, 1100, or 1000 OD/ml).

In some embodiments, the mixture includes a nucleic acid concentration (e.g., overall nucleic acid concentration, double-stranded nucleic acid concentration, or single-stranded nucleic acid concentration) of at least about 900 OD/mL (e.g., about 900 OD/mL).

In some embodiments, the mixture includes a nucleic acid concentration (e.g., overall nucleic acid concentration, double-stranded nucleic acid concentration, or single-stranded nucleic acid concentration) of at least about 1100 OD/mL (e.g., about 1100 OD/mL).

In some embodiments, the mixture includes a nucleic acid concentration (e.g., overall nucleic acid concentration, double-stranded nucleic acid concentration, or single-stranded nucleic acid concentration) of at least about 400 OD/mL (e.g., about 400 OD/mL).

In some embodiments, the mixture includes an amount of the single-stranded nucleic acid in excess of the amount of the double-stranded nucleic acid. In some embodiments, the amount of the single-stranded nucleic acid is in excess relative to the amount of the double-stranded nucleic acid by at least about 1%, 2%, 3%, 4%, 5%, 6%, 7%, 8%, 9%, 10%, 15%, 20%, 25%, 30%, 40%, 50%, 60%, 70%, 80%, or 90%. In particular embodiments, the amount of the single-stranded nucleic acid is in excess relative to the amount of the double-stranded nucleic acid by about 5%.

In some embodiments, the concentration of a salt (e.g., a cation, e.g., sodium ion) in the mixture is less than about 10, 20, 30, 40, 50, 60, 70, 80, 90, 100, 200, 300, 400, 500, 600, 700, 800, 900, 1000, 1500, 2000, or 3000 mM. In some embodiments, the mixture does not substantially comprise the salt. In some embodiments, the concentration of a salt (e.g., a cation, e.g., sodium ion) in the mixture is about 50 mM to about 1.5 M, e.g., about 60 nM to about 1.2 M or about 69 mM to about 1.02 M. In some embodiments, the single-stranded oligonucleotides are in a deprotection sample matrix. In some embodiments, the melting temperature of the double-stranded nucleic acid in the mixture is between about 20-85° C., e.g., between about 25-80, 30-75, 35-70, 40-65, 45-60, 20-25, 25-30, 30-35, 35-40, 40-45, 45-50, 50-55, 55-60, 60-65, 65-70, 70-75, 75-80, or 80-85° C.

D. Diafiltration total volume and time

Without wishing to be bound by theory, it is contemplated that the diafiltration total volume (DTV) and/or time of the apparatus, the pressure at which the mixture is ultrafiltered, and the temperature at which the mixture is ultrafiltered are each process parameters that can be important for determining the efficacy of a method of duplex oligonucleotide purification. Together, these process parameters comprise a combination of process parameters whose optimal configurations relate, e.g., depend upon, each other.

In some embodiments, the ultrafiltration step includes applying at least 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, or 90 diafiltration volumes (DTVs) to the filtration apparatus (e.g., sequentially). In some embodiments, the ultrafiltration step includes no more than 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, or 90 DTVs. In some embodiments, the ultrafiltration step comprises applying at least 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, or 90 diafiltration volumes (DTVs) to the filtration apparatus (e.g., sequentially) and optionally no more than 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, or 90 DTVs. In some embodiments, the ultrafiltration step comprises applying 20-90, 25-90, 30-90, 35-90, 40-90, 45-90, 50-90, 55-90, 60-90, 65-90, 70-90, 75-90, 80-90, 85-90, 20-85, 25-85, 30-85, 35-85, 40-85, 45-85, 50-85, 55-85, 60-85, 65-85, 70-85, 75-85, 80-85, 20-80, 25-80, 30-80, 35-80, 40-80, 45-80, 50-80, 55-80, 60-80, 65-80, 70-80, 75-80, 20-75, 25-75, 30-75, 35-75, 40-75, 45-75, 50-75, 55-75, 60-75, 65-75, 70-75, 20-70, 25-70, 30-70, 35-70, 40-70, 45-70, 50-70, 55-70, 60-70, 65-70, 20-65, 25-65, 30-65, 35-65, 40-65, 45-65, 50-65, 55-65, 60-65, 20-60, 25-60, 30-60, 35-60, 40-60, 45-60, 50-60, 55-60, 20-55, 25-55, 30-55, 35-55, 40-55, 45-55, 50-55, 20-50, 25-50, 30-50, 35-50, 40-50, 45-50, 20-45, 25-45, 30-45, 35-45, 40-45, 20-40, 25-40, 30-40, 35-40, 20-35, 25-35, 30-35, 20-30, 25-30, or 20-25 DTVs to the filtration apparatus (e.g., sequentially). In some embodiments, the ultrafiltration step comprises applying 20-90, 25-90, 30-90, 35-90, 40-90, 45-90, 50-90, 55-90, 60-90, 65-90, 70-90, 75-90, 80-90, 85-90, 20-85, 25-85, 30-85, 35-85, 40-85, 45-85, 50-85, 55-85, 60-85, 65-85, 70-85, 75-85, 80-85, 20-80, 25-80, 30-80, 35-80, 40-80, 45-80, 50-80, 55-80, 60-80, 65-80, 70-80, 75-80, 20-75, 25-75, 30-75, 35-75, 40-75, 45-75, 50-75, 55-75, 60-75, 65-75, 70-75, 20-70, 25-70, 30-70, 35-70, 40-70, 45-70, 50-70, 55-70, 60-70, 65-70, 20-65, 25-65, 30-65, 35-65, 40-65, 45-65, 50-65, 55-65, 60-65, 20-60, 25-60, 30-60, 35-60, 40-60, 45-60, 50-60, 55-60, 20-55, 25-55, 30-55, 35-55, 40-55, 45-55, 50-55, 20-50, 25-50, 30-50, 35-50, 40-50, 45-50, 20-45, 25-45, 30-45, 35-45, 40-45, 20-40, 25-40, 30-40, 35-40, 20-35, 25-35, 30-35, 20-30, 25-30, or 20-25 DTVs to the filtration apparatus (e.g., sequentially).

In some embodiments, the ultrafiltration step includes applying DTVs to the filtration apparatus (e.g., sequentially) until a steady low conductivity threshold is achieved. In some embodiments, the ultrafiltration is performed until a steady low conductivity threshold is achieved (e.g., a conductivity threshold of less than or equal to about 30, 40, 50, 60, 70, or 80 μS/cm, e.g., about 50 μS/cm), e.g., DTVs are applied until a steady low conductivity threshold is achieved.

### E. Pressure

Without wishing to be bound by theory, it is contemplated that the pressure at which the mixture is ultrafiltered, the diafiltration total volume (DTV) and/or time of the apparatus, and the temperature at which the mixture is ultrafiltered are each process parameters that are important for determining the efficacy of a method of duplex oligonucleotide purification. Together, these process parameters comprise a combination of process parameters whose optimal configurations relate, e.g., depend upon, each other.

In some embodiments, the ultrafiltration step includes applying a force of at least 5, 6, 7, 8, 9, 10, 11, 12, 13, 14, 15, 16, 17, 18, 19, 20, 21, 22, 23, 24, 25, 26, 27, 28, or 29 psi (transmembrane pressure) to the double-stranded nucleic acid product mixture, e.g., within the filtration apparatus. In some embodiments, the ultrafiltration step includes applying a force that is no more than 30, 29, 28, 27, 26, 25, 24, 23, 22, 21, 20, 19, 18, 17, 16, 15, 14, 13, 12, 11, 10, 9, 8, 7, or 6 psi to the double-stranded nucleic acid product mixture, e.g., within the filtration apparatus. In some embodiments, the ultrafiltration step includes applying a force of at least 5, 6, 7, 8, 9, 10, 11, 12, 13, 14, 15, 16, 17, 18, 19, 20, 21, 22, 23, 24, 25, 26, 27, 28, or 29 psi (transmembrane pressure) and optionally no more than 30, 29, 28, 27, 26, 25, 24, 23, 22, 21, 20, 19, 18, 17, 16, 15, 14, 13, 12, 11, 10, 9, 8, 7, or 6 psi to the double-stranded nucleic acid product mixture, e.g., within the filtration apparatus. In some embodiments, the ultrafiltration step includes applying a force of 5-30, 10-30, 15-30, 20-30, 25-30, 5-25, 10-25, 15-25, 20-25, 5-20, 10-20, 15-20, 5-15, 10-15, or 5-10 psi (transmembrane pressure). In some embodiments, the ultrafiltration step includes applying a force within the filtration apparatus' manufacturer's recommended pressure operating conditions.

In some embodiments, the ultrafiltration step includes achieving a recirculation rate under pressure of at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39 L/min/m² of the membrane. In some embodiments, the ultrafiltration step includes achieving a recirculation rate under pressure of no more than 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, or 6 L/min/m² of the membrane. In some embodiments, the ultrafiltration step includes achieving a recirculation rate under pressure of at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39 L/min/m² and optionally no more than 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, or 6 L/min/m² of the membrane.

In some embodiments, the ultrafiltration step includes achieving a recirculation rate under pressure of at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39 L/min/m² of the membrane. In some embodiments, the ultrafiltration step includes achieving a recirculation rate under pressure of no more than 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, or 6 L/min/m² of the membrane. In some embodiments, the ultrafiltration step includes achieving a recirculation rate under pressure of at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39 L/min/m² and optionally no more than 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, or 6 L/min/m² of the membrane. In some embodiments, the ultrafiltration step includes achieving a recirculation rate under pressure of at least 5, 6, 7, 8, 9, 10, 11, or 12 L/min/m² of the membrane.

In some embodiments the ultrafiltration step comprises achieving a recirculation rate under pressure of 5-40, 10-40, 15-40, 20-40, 25-40, 30-40, 35-40, 5-35, 10-35, 15-35, 20-35, 25-35, 30-35, 5-30, 10-30, 15-30, 20-30, 25-30, 5-25, 10-25, 15-25, 20-25, 5-20, 10-20, 15-20, 5-15, 10-15, or 5-10 L/min/m².

### F. Temperature

Without wishing to be bound by theory, it is thought that the temperature at which the mixture is ultrafiltered, the pressure at which the mixture is ultrafiltered, and the diafiltration total volume (DTV) and/or time of the apparatus are each process parameters that are important for determining the efficacy of a method of duplex oligonucleotide purification. Together, these process parameters comprise a combination of process parameters whose optimal configurations relate, e.g., depend upon, each other.

In some embodiments, the ultrafiltration is performed at a temperature of about 10-40° C., e.g., about 15-40, 20-40, 25-40, 30-40, 35-40, 10-35, 10-30, 10-25, 10-20, 10-15, 15-35, 20-30, 10-15, 15-20, 20-25, 25-30, 30-35, or 35-40° C., e.g., about 10, 15, 20, 25, 30, 35, 37, or 40° C. In some embodiments, the ultrafiltration is performed at a temperature that is at least 0.5° C., 1° C., 1.5° C., 2° C., 2.5° C., 3° C., 3.5° C., 4° C., 5° C., 10° C., 15° C., 20° C., 25° C., 30° C., 35° C., 40° C., or 45° C. less than the point of oligo denaturation. In some embodiments, the ultrafiltration is performed at a temperature that never reaches or exceeds (e.g., is less than) the point of oligo denaturation anywhere in the system. In some embodiments, the temperature at which the ultrafiltration is performed does not vary by more than about 0° C., 1° C., 2° C., 3° C., 4° C., or 5° C. over the course of the filtration.

### IV. EXAMPLES

The invention is further illustrated by the following examples. The examples are provided for illustrative purposes only and are not to be construed as limiting the scope or content of the invention in any way.

### Example 1—Exemplary Purification Method

In this example, a duplex oligonucleotide comprised of two sufficiently complementary strands is formed synthetically by annealing together equal molar amounts of the individual strands. Analysis by non-denaturing HPLC is used to measure the content for duplex and unreacted single strands. Analysis of the duplex oligonucleotide samples is performed using an Agilent 1290 UHPLC equipped with a Waters XBridge BEH C4 300A, 2.1×50 mm, 3.5 uM analytical column run at 35° C. The peaks are eluted with Mobile Phase A: 95 mM 1,1,1,3,3,3-hexafluoro-2-propanol, 16 mM triethylamine, 24 mM t-butylamine and 1 μM ethylenediaminetetraacetic acid, and Mobile Phase B: Acetonitrile over 26.25 minutes to compare excess single strand and duplex abundances.

### Example 2—Exemplary Ultrafiltration or Crossflow Filtration

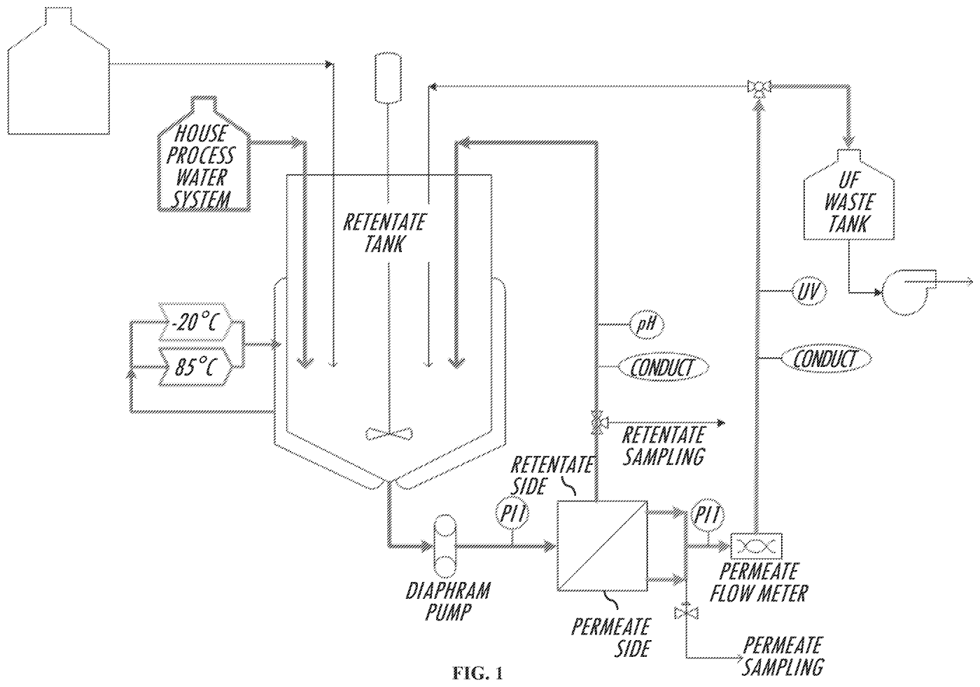

Oligonucleotide matrix is introduced to an ultrafiltration or crossflow ultrafiltration apparatus. Standard ultrafiltration equipment is employed that consists of a retentate tank, pump, ultrafiltration membrane with specific molecular weight retention aspect (e.g. 2, 5, 10 kD), conductivity meter and a waste vessel (FIG. 1). The system is pressurized at medium psi range (˜15-30 psi) to concentrate the matrix on the retentate side of the permeable membrane. Process water is then introduced under pressure to facilitate diafiltration of low molecular weight contaminants.

Diafiltration is continued until a steady low conductivity threshold is achieved, typically (<50 μS/cm) and to a minimum target of system volume exchanges.

### Example 3—Duplex Nucleic Acid>10 kDa Molecular Weight at ˜900 OD/mL Concentration

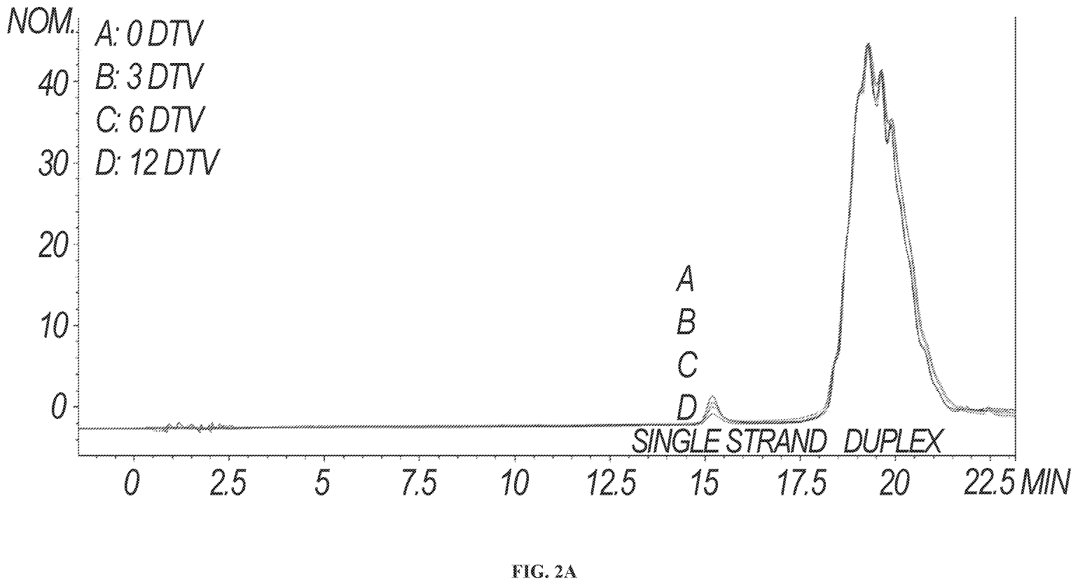

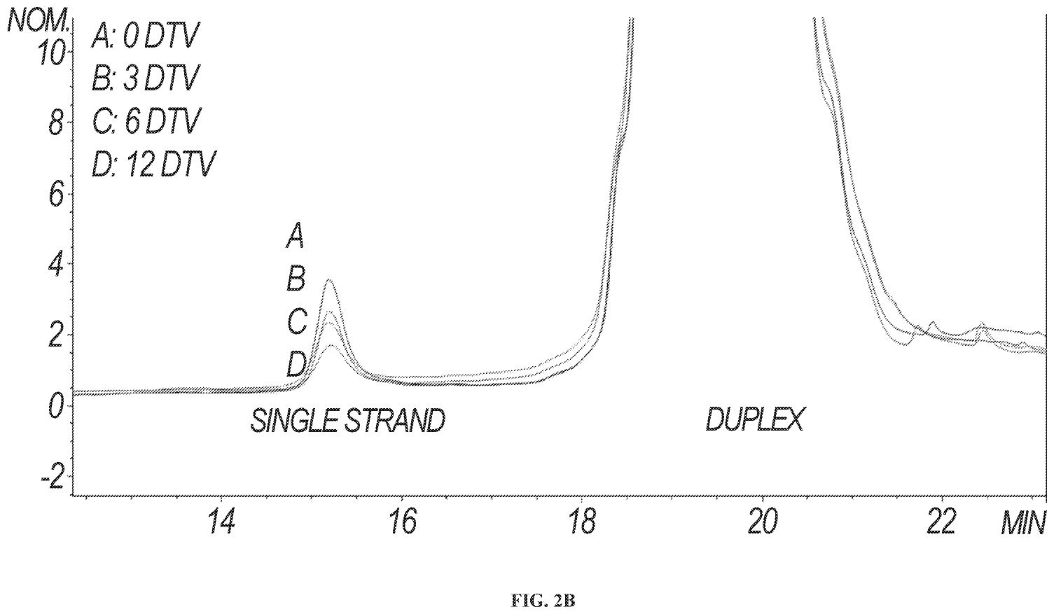

An ultrafiltration apparatus was equipped with a 10 kDa molecular weight cut off membrane (HYDROSART® Ultrafilter, Sartorius). The duplex oligonucleotide of >10 kDa molecular weight containing an excess of ˜5% single strand and ˜900 OD/mL total oligonucleotide concentration was ultrafiltered as described in Example 1 and Example 2. Diafiltration was performed as a function of Diafiltration Total Volume (DTV) and time. Samples of the retentate matrix were analyzed as a function of time by UV and non-denaturing HPLC for composition of duplex and single strand. As shown in Table 1, below, and FIGS. 2A-2C, the percentage of the lower molecular weight single strand relative to the percent duplex decreased over time.

TABLE 1
HPLC data of Example 2

| Sample | Single-Stranded | Duplex |
|---|---|---|
| T = 0 | 2.0 | 98.0 |
| DTV1 | 1.7 | 98.3 |
| DTV2 | 1.7 | 98.3 |
| DTV3 | 1.6 | 98.4 |
| DTV4 | 1.5 | 98.5 |
| DTV5 | 1.3 | 98.7 |
| DTV6 | 1.3 | 98.7 |
| DTV9 | 0.9 | 99.1 |
| DTV12 | 0.8 | 99.2 |
| DTV15 | 0.7 | 99.3 |

### Example 4—Duplex Nucleic Acid>10 kDa Molecular Weight at ˜400 OD/mL Concentration

An ultrafiltration apparatus was equipped with a 10 kDa molecular weight cut off membrane. The duplex oligonucleotide of >10 kDa molecular weight containing an excess of ˜5% single strand and ˜400 OD/mL oligonucleotide concentration was ultrafiltered as described in Example 1 and Example 2. Diafiltration was performed as a function of Diafiltration Total Volume (DTV) and time. Samples of the retentate matrix were analyzed as a function of time by UV and non-denaturing HPLC for composition of duplex and single strand. The percentage of the lower molecular weight single strand relative to the percent duplex did not decrease over time, as shown in Table 2, below.

TABLE 2
HPLC data of Example 3

| Sample | Single-Stranded | Duplex |
|---|---|---|
| T = 0 | 5.1 | 90.2 |
| DTV3 | 5.1 | 90.4 |
| DTV5 | 5.0 | 90.5 |
| DTV8 | 5.0 | 90.5 |
| DTV11 | 5.0 | 90.5 |
| DTV14 | 4.9 | 90.7 |

### Example 5—Duplex Nucleic Acid>10 kDa Molecular Weight at ˜1100 OD/mL Concentration

Ultrafiltration apparatus was equipped with a 10 kDa molecular weight cut off membrane. The duplex oligonucleotide of >10 kDa molecular weight containing an excess of ˜5% single strand and 1,100-1,400 OD/mL oligonucleotide concentration was ultrafiltered as described in Example 1 and Example 2. Diafiltration was performed as a function of Diafiltration Total Volume (DTV) and time. Samples of the retentate matrix were analyzed as a function of time by UV and non-denaturing HPLC for composition of duplex and single strand. The percentage of the lower molecular weight single strand relative to the percent duplex decreased over time, as shown in Table 3, below.

TABLE 3
HPLC data of Example 3

| Sample | Single-Stranded | Duplex |
|---|---|---|
| T = 0 | 5.3 | 90.1 |
| DTV1 | 4.7 | 91.1 |
| DTV2 | 4.8 | 91.2 |
| DTV3 | 4.9 | 91.3 |
| DTV7 | 3.5 | 92.0 |
| DTV11 | 3.4 | 92.1 |
| DTV27 | 3.2 | 92.0 |
| DTV45 | 3.0 | 92.3 |
| DTV63 | 2.8 | 92.8 |
| DTV81 | 2.7 | 93.0 |

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

1. A method of separating a double-stranded nucleic acid from a single-stranded nucleic acid, comprising:
providing a mixture of double-stranded nucleic acid and single-stranded nucleic acid, and
subjecting the mixture to an ultra-filtration step that selectively retains double-stranded nucleic acid and not single-stranded nucleic acid,
wherein the ultra-filtration step comprises applying the mixture to a filtration apparatus, wherein the filtration apparatus comprises a membrane with (i) a molecular weight (MW) cutoff that is at least 10% less than the MW of the double-stranded nucleic acid and at least 10% more than the MW of the single-stranded nucleic acid, and/or (ii) a pore size that will allow at least 90% of the single-stranded nucleic acids and at most 10% of the double-stranded nucleic acids to permeate the membrane,
thereby separating a double-stranded nucleic acid from a single-stranded nucleic acid.

2. An apparatus comprising:
a first chamber configured to hold a mixture to be filtered, and optionally to capture retentate, a second chamber configured to capture permeate, and a filtration element disposed between the first and second chambers;
wherein the mixture comprises double-stranded nucleic acid and single-stranded nucleic acid;
wherein the filtration element selectively retains double-stranded nucleic acid and not single-stranded nucleic acid, optionally wherein the filtration element comprises a membrane with (i) a molecular weight (MW) cutoff that is at least 10% less than the MW of the double-stranded nucleic acid and at least 10% more than the MW of the single-stranded nucleic acid, and/or (ii) a pore size that will allow at least 90% of the single-stranded nucleic acids and at most 10% of the double-stranded nucleic acids to permeate the membrane.

3. The method of claim 1, wherein the ultra-filtration step or filtration element also does not retain one or more other mixture component, wherein the other mixture component is selected from the group consisting of: a shorter length single strand nucleic acid, an organic solvent, a thiolation reagent/by-product, a capping reagent, a coupling reagent, a conjugation reagent, a detritylation reagent/by-product, a deprotection reagent/by-product, a carbohydrate, a peptide, a lipid, a polyethylene glycol, or a fluorescent label.

4. The method of claim 1, wherein the ultra-filtration step produces a retentate and a permeate, and wherein the retentate comprises a different concentration of one or more of a salt, a buffer, a deprotection base, or a production reagent or byproduct than the permeate, the mixture, or both.

5. The method of claim 1, wherein providing a mixture comprises:
providing a first single-stranded nucleic acid and a second single-stranded nucleic acid which comprise sequences that are sufficiently complementary to one another to hybridize under conditions suitable for hybridization, and
combining the first single-stranded nucleic acid and second single-stranded nucleic acid under conditions suitable for hybridization to produce a mixture comprising:
a double-stranded nucleic acid comprising a first single-stranded nucleic acid and a second single-stranded nucleic acid, and
the first single-stranded nucleic acid in single-stranded form, the second single-stranded nucleic acid in single-stranded form, or both.

6. The method of claim 1, wherein the first single-stranded nucleic acid, second single-stranded nucleic acid, or both are or comprise DNA, RNA, UNA, PNA, or LNA.

7. The method of claim 1, wherein the first single-stranded nucleic acid, second single-stranded nucleic acid, or both comprise one or more modified and/or non-canonical nucleotides chosen from: MOE, 2′fluoro, 2′OMe, 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, 2-thiocytidine, methylated bases, intercalated bases, a nucleotide comprising a modified sugar, or any combination thereof.

8. The apparatus of claim 2, wherein the filtration element comprises a cross-flow filter.

9. The method of claim 1, wherein the ultrafiltration step comprises applying a force of 5-30 psi (transmembrane pressure).

10. The method of claim 1, wherein the ultrafiltration step comprises applying 20-90 DTVs to the filtration apparatus.

11. The method of claim 1, wherein the ultrafiltration is performed until a steady low conductivity threshold is achieved.

12. The method of claim 1, wherein the double-stranded nucleic acid comprises a conjugate group.

13. The method of claim 1, wherein the ultra-filtration step comprises diafiltration.

14. The method of claim 13, wherein the diafiltration is performed a period of sufficient to filter at least about 2, 3, 4, 5, 6, 7, 8, 9, 10, 11, 12, 13, 14, 15, 20, 25, 27, 30, 35, 40, 45, 50, 55, 60, 63,65, 70, 75, 80, 81, 85, or 90 diafiltration volumes (DTVs).

15. The method of claim 1, wherein the ultra-filtration is applied at a pressure of about 5-40 psi.

16. The method of claim 1, wherein the ultra-filtration is performed at a temperature of about 10-40° C.

17. The method of claim 1, wherein the mixture comprises a nucleic acid concentration of at least 100 OD/mL.

18. The method of claim 1, wherein the ratio of double-stranded nucleic acid relative to single-stranded nucleic acid in the retentate increases over time and/or over diafiltration total volume (DTV).

19. The method of claim 1, wherein the method reduces the level of the double-stranded nucleic acid in the retentate as compared to the mixture by no more than 0.1, 0.5, 1, 2, 3, 4, 5, 6, 7, 8, 9, or 10%.

20. A method of optimizing a technique for manufacturing a purified double-stranded nucleic acid, comprising:
(i) providing a mixture comprising:
a double-stranded nucleic acid comprising a first single-stranded nucleic acid and a second single-stranded nucleic acid at a first concentration, and
a single-stranded nucleic acid;
wherein the mixture has a predetermined concentration of a salt;
(ii) subjecting the mixture to an ultra-filtration step that selectively retains double-stranded nucleic acid comprising the first and second single-stranded nucleic acids and not the single-stranded nucleic acid, wherein the ultra-filtration step comprises applying the mixture to a filtration apparatus with a predetermined molecular weight cutoff for a predetermined length of time;
(iii) harvesting the retentate;
(iv) varying one of or more of the first concentration, the second concentration, the predetermined molecular weight cutoff, the predetermined length of time, or the salt concentration;
(v) repeating steps (i)-(iii) one or more times, each using one or more of the varied first concentration, second concentration, predetermined molecular weight cutoff, predetermined length of time, and/or salt concentration;
(vi) determining, for each repetition of steps (i)-(iii), the ratio of double-stranded nucleic acid to single-stranded nucleic acid in the harvested retentates; and
(vii) identifying a first concentration, the second concentration, predetermined molecular weight cutoff, predetermined length of time, and/or salt concentration that yields the highest ratio of double-stranded nucleic acid to single-stranded nucleic acid in the harvested retentate;
thereby optimizing a technique for manufacturing a purified double-stranded nucleic acid.
